(19) United States
(12) Patent Application Publication
(10) Pub. No.: US 2025/0197418 A1
(43) Pub. Date: Jun. 19, 2025

(54) Polymorphic Compounds And Uses Thereof

(71) Applicant: Prelude Therapeutics Incorporated, Wilmington, DE (US)

(72) Inventors: Bo Shen, Wilmington, DE (US); Ganfeng Cao, Wilimington, DE (US); Andrew Combs, Wilmington, DE (US); Chaofeng Dai, Wilmington, DE (US); Patrick Wen, Wilmington, DE (US); Andrew Buesking, Wilmington, DE (US); Reddy Perumalla Sathyanarayana, Wilmington, DE (US); Yufeng Wei, Nanjing, Jiangsu (CN); Pengpeng Ye, Nanjing, Jiangsu (CN)

(21) Appl. No.: 18/849,602

(22) PCT Filed: Mar. 23, 2022
(86) PCT No.: PCT/CN2022/082452

§ 371 Date: Sep. 23, 2024

(51) Int. Cl. C07D 495/04; A61K 31/4196; A61K 31/506; A61K 31/565; A61K 45/06

(52) U.S. Cl. CPC C07D 495/04; A61K 31/4196; A61K 31/506; A61K 31/565; A61K 45/06

(57) ABSTRACT

The present invention provides freebase and salt forms useful for treating various CDK4-mediated and a CDK6-mediated disorders, such as cancer, by the administration of small molecule therapeutics that act as inhibitors of CDK4 and CDK6. Pharmaceutical compositions comprising the freebase and salt forms, as well as methods of their use and preparation, are also described.

### FIELD OF THE INVENTION

The disclosure is directed to CDK inhibitors and methods of their use.

### BACKGROUND OF THE INVENTION

Cyclin-dependent kinases (CDKs) are a family of conserved serine/threonine kinases that play critical roles in cell cycle and gene transcription regulation (Malumbres 2014). Among the cell cycle CDK subfamily, CDK4 and CDK6 are the master regulators that control entry of cells from the first gap phase (G1) to the DNA synthesis phase(S). During this process, cyclin D protein levels increase, complex with CDK4/6 and activate their kinase activities. Activated CDK4/6 complexes phosphorylate retinoblastoma protein (RB1) and other RB1-like proteins, reduce their binding affinities and release RB1-containing transcription repressor complexes from E2F transcription factors, resulting in activation of E2F controlled cell cycle genes and progression of cell cycle (Lapenna and Giordano 2009, Asghar, Witkiewicz et al. 2015).

Given the central roles CDK4/6 play in cell cycle regulation, disfunction of which is a hallmark of cancer (Hanahan and Weinberg 2011), dysregulation of CDK4/6 pathway has been frequently observed in cancer, such as (epi) genetic inactivation of endogenous CDK4/6 inhibitor p16INK4A and amplification/overexpression of CDK4/6 as well as cyclin D proteins (Lapenna and Giordano 2009, Malumbres and Barbacid 2009, Asghar, Witkiewicz et al. 2015, O'Leary, Finn et al. 2016). CDK4/6 have been intensively investigated as potential therapeutic targets for cancer treatment and the recent approval of CDK4/6 selective inhibitors, namely, Palbociclib (U.S. Food & Drug Administration. 2017), Ribociclib (U.S. Food & Drug Administration. 2017), and Abemaciclib (U.S. Food & Drug Administration. 2018), in combination with endocrine therapies, to treat hormone receptor (HR) positive and human epidermal growth factor receptor 2 (HER2) negative metastatic breast cancer further validated this thesis.

Central nervous system (CNS) diseases such as glioblastoma (GBM) and brain metastases are challenging malignancies with urgent unmet needs. GBM is the most common and aggressive primary brain cancer in adults with overall 5-year survival rate less than 6% (Ostrom, Gittleman et al. 2016). Large scale genomic studies revealed that the cyclin D-CDK4/6-RB1 pathway is alternated in majority of gliomas and represents one of the most perturbed pathways (Cancer Genome Atlas Research 2008, Brennan, Verhaak et al. 2013), suggesting CDK4/6 may be good targets for GBM. Brain metastases, on the other hand, may arise from an estimated of 20% of all cancer patients but still lacks effective treatments (Achrol, Rennert et al. 2019). Interestingly, genomic studies also identified CDK pathway as one of three most altered and actionable genetic alternations in brain metastases (Brastianos, Carter et al. 2015, Valiente, Ahluwalia et al. 2018). However, despite positive preclinical data supporting targeting CDK4/6 to treat GBM (Yin, Li et al. 2018, Bronner, Merrick et al. 2019), and initial signs of brain penetration of Abemaciclib in patients (Patnaik, Rosen et al. 2016, Sahebjam, Rhun et al. 2016), clinical development of CDK4/6 inhibitors in the clinic for GBM or brain metastases are still in early stage or unsuccessful (Anders, Rhun et al. 2019, Nguyen, Searle et al. 2019, Sahebjam, Le Rhun et al. 2019), likely due to their inability to penetrate the blood-brain barrier (BBB) (de Gooijer, Zhang et al. 2015, Parrish, Pokorny et al. 2015, Raub, Wishart et al. 2015).

Additional small molecule CDK4/6 inhibitors are needed.

### SUMMARY OF THE INVENTION

It has now been found that compounds of the present invention, and compositions thereof, are useful for treating, preventing, and/or reducing a risk of a disease, disorder, or condition in a CDK4-mediated and a CDK6-mediated disorder. Such compounds are represented by the chemical structure below, denoted as compound A:

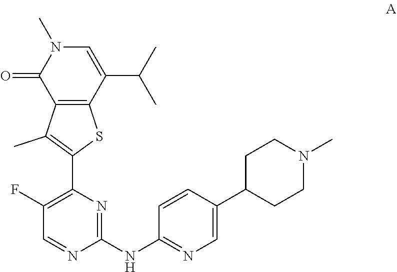 [SMILES: Cc1c(-c2nc(Nc3ccc(C4CCN(C)CC4)cn3)ncc2F)sc2c(C(C)C)cn(C)c(=O)c12]

or a pharmaceutically acceptable salt thereof.

Compounds of the present invention, and pharmaceutically acceptable compositions thereof, are useful for treating a variety of diseases, disorders or conditions, associated with CDK4-mediation and a CDK6-mediation. Such diseases, disorders, or conditions include those described herein.

### BRIEF DESCRIPTION OF THE FIGURES

FIG. 1 depicts the XRPD pattern of Compound A, free base Form A.

FIG. 2 depicts a DSC thermogram and TGA trace of Compound A, free base Form A.

FIG. 3 depicts the XRPD pattern of Compound A, free base Form B.

FIG. 4 depicts a DSC thermogram and TGA trace of Compound A, free base Form B.

FIG. 5 depicts the XRPD pattern of Compound A, free base Form C.

FIG. 6 depicts a DSC thermogram and TGA trace of Compound A, free base Form C.

FIG. 7 depicts the XRPD pattern of Compound A, succinate salt Form A.

FIG. 8 depicts a DSC thermogram and TGA trace of Compound A, succinate salt Form A.

FIG. 9 depicts the XRPD pattern of Compound A, succinate salt Form B.

FIG. 10 depicts a DSC thermogram and TGA trace of Compound A, succinate salt Form B.

FIG. 11 depicts the XRPD pattern of Compound A, succinate salt Form C.

FIG. 12 depicts a DSC thermogram and TGA trace of Compound A, succinate salt Form C.

FIG. 13 depicts the XRPD pattern of Compound A, succinate salt Form D.

FIG. 14 depicts a DSC thermogram and TGA trace of Compound A, succinate salt Form D.

FIG. 15 depicts the XRPD pattern of Compound A, succinate salt Form E.

FIG. 16 depicts a DSC thermogram and TGA trace of Compound A, succinate salt Form E.

FIG. 17 depicts the XRPD pattern of Compound A, succinate salt Form F.

FIG. 18 depicts a DSC thermogram and TGA trace of Compound A, succinate salt Form F.

FIG. 19 depicts a DSC thermogram and TGA traceof Compound A, succinate salt Form F.

FIG. 20 depicts the XRPD pattern of Compound A, succinate salt Form G.

FIG. 21 depicts a DSC thermogram and TGA trace of Compound A, succinate salt Form G.

FIG. 22 depicts a DSC thermogram and TGA trace of Compound A, succinate salt Form G.

FIG. 23 depicts a single crystal of Compound A, succinate salt Form G.

FIG. 24 depicts the XRPD pattern of Compound A, hydrochloride salt Form A.

FIG. 25 depicts a DSC thermogram and TGA trace of Compound A, hydrochloride salt Form A.

FIG. 26 depicts the XRPD pattern of Compound A, hydrochloride salt Form B.

FIG. 27 depicts a DSC thermogram and TGA trace of Compound A, hydrochloride salt Form B.

FIG. 28 depicts the XRPD pattern of Compound A, hydrochloride salt Form C.

FIG. 29 depicts a DSC thermogram and TGA trace of Compound A, hydrochloride salt Form C.

FIG. 30 depicts the XRPD pattern of Compound A, hydrochloride salt Form D.

FIG. 31 depicts a DSC thermogram and TGA trace of Compound A, hydrochloride salt Form D.

FIG. 32 depicts the XRPD pattern of Compound A, hydrochloride salt Form E.

FIG. 33 depicts a DSC thermogram and TGA trace of Compound A, hydrochloride salt Form E.

FIG. 34 depicts the XRPD pattern of Compound A, hydrochloride salt Form F.

FIG. 35 depicts a DSC thermogram and TGA trace of Compound A, hydrochloride salt Form F.

FIG. 36 depicts the XRPD pattern of Compound A, maleate salt Form A.

FIG. 37 depicts a DSC thermogram and TGA trace of Compound A, maleate salt Form A.

FIG. 38 depicts the XRPD pattern of Compound A, sulfate salt Form A.

FIG. 39 depicts a DSC thermogram and TGA trace of Compound A, sulfate salt Form A.

FIG. 40 depicts the XRPD pattern of Compound A, sulfate salt Form B.

FIG. 41 depicts a DSC thermogram and TGA trace of Compound A, sulfate salt Form B.

FIG. 42 depicts the XRPD pattern of Compound A, sulfate salt Form C.

FIG. 43 depicts a DSC thermogram and TGA trace of Compound A, sulfate salt Form C.

FIG. 44 depicts the XRPD pattern of Compound A, phosphate salt Form A.

FIG. 45 depicts a DSC thermogram and TGA trace of Compound A, phosphate salt Form A.

FIG. 46 depicts the XRPD pattern of Compound A, L-tartrate salt Form A.

FIG. 47 depicts a DSC thermogram and TGA trace of Compound A, L-tartrate salt Form A.

FIG. 48 depicts the XRPD pattern of Compound A, fumarate salt Form A.

FIG. 49 depicts a DSC thermogram and TGA trace of Compound A, fumarate salt Form A.

FIG. 50 depicts the XRPD pattern of Compound A, fumarate salt Form B.

FIG. 51 depicts a DSC thermogram and TGA trace of Compound A, fumarate salt Form B.

FIG. 52 depicts the XRPD pattern of Compound A, fumarate salt Form C.

FIG. 53 depicts a DSC thermogram and TGA trace of Compound A, fumarate salt Form C.

FIG. 54 depicts the XRPD pattern of Compound A, fumarate salt Form D.

FIG. 55 depicts a DSC thermogram and TGA trace of Compound A, fumarate salt Form D.

FIG. 56 depicts the XRPD pattern of Compound A, citrate salt Form A.

FIG. 57 depicts a DSC thermogram and TGA trace of Compound A, citrate salt Form A.

FIG. 58 depicts the XRPD pattern of Compound A, L-malate salt Form A.

FIG. 59 depicts a DSC thermogram and TGA trace of Compound A, L-malate salt Form A.

FIG. 60 depicts the XRPD pattern of Compound A, mesylate salt Form A.

FIG. 61 depicts a DSC thermogram and TGA trace of Compound A, mesylate salt Form A.

FIG. 62 depicts the XRPD pattern of Compound A, mesylate salt Form B.

FIG. 63 depicts a DSC thermogram and TGA trace of Compound A, mesylate salt Form B.

FIG. 64 depicts the XRPD pattern of Compound A, esylate salt Form A.

FIG. 65 depicts a DSC thermogram and TGA trace of Compound A, esylate salt Form A.

FIG. 66 depicts the XRPD pattern of Compound A, esylate salt Form B.

FIG. 67 depicts a DSC thermogram and TGA trace of Compound A, esylate salt Form B.

FIG. 68 depicts the XRPD pattern of Compound A, tosylate salt Form A.

FIG. 69 depicts a DSC thermogram and TGA trace of Compound A, tosylate salt Form A.

FIG. 70 depicts the XRPD pattern of Compound A, tosylate salt Form B.

FIG. 71 depicts a DSC thermogram and TGA trace of Compound A, tosylate salt Form B.

FIG. 72 depicts the XRPD pattern of Compound A, glutarate salt Form A.

FIG. 73 depicts the XRPD pattern of Compound A, acetate salt Form A.

FIG. 74 depicts a DSC thermogram and TGA trace of Compound A, acetate salt Form A.

FIG. 75 depicts the XRPD pattern of Compound A, malonate salt Form A.

FIG. 76 depicts the XRPD pattern of Compound A, malonate salt Form B.

FIG. 77 depicts the XRPD pattern of Compound A, sebacate salt Form A.

FIG. 78 depicts a DSC thermogram and TGA trace of Compound A, sebacate salt Form A.

FIG. 79 depicts the XRPD pattern of Compound A, aceturate salt Form A.

FIG. 80 depicts a DSC thermogram and TGA trace of Compound A, aceturate salt Form A.

FIG. 81 depicts the XRPD pattern of Compound A, aceturate salt Form B.

FIG. 82 depicts a DSC thermogram and TGA trace of Compound A, aceturate salt Form B.

### DETAILED DESCRIPTION OF THE INVENTION

### General Description of Certain Aspects of the Invention

U.S. patent application Ser. No. 17/480,323 (hereinafter “the '323 application”), filed Sep. 21, 2021, the entirety of which is hereby incorporated herein by reference, describes certain CDK4-inhibitors and CDK6-inhibitors. Such compounds include compound A:

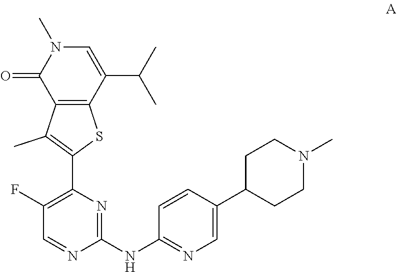 [SMILES: Cc1c(-c2nc(Nc3ccc(C4CCN(C)CC4)cn3)ncc2F)sc2c(C(C)C)cn(C)c(=O)c12]

Compound A, 2-(5-Fluoro-2-((5-(1-methylpiperidin-4-yl) pyridin-2-yl)amino)-pyrimidin-4-yl)-7-isopropyl-3,5-dimethylthieno[3,2-c]pyridin-4 (5H)-one, is set forth in Example 56 of the '323 application and the synthesis of compound A is described in detail at Example 56, and is reproduced herein for ease of reference.

It would be desirable to provide a solid form of compound A (e.g., as a freebase thereof or salt thereof) that imparts characteristics such as improved aqueous solubility, stability and ease of formulation. Accordingly, the present invention provides both free base forms and salt forms of compound A.

### Free Base Forms of Compound A

It is contemplated that compound A free base can exist in a variety of physical forms. For example, compound A free base can be in solution, suspension, or in solid form. In certain embodiments, compound A free base is in solid form. When compound A free base is in solid form, said compound may be amorphous, crystalline, or a mixture thereof. Exemplary solid forms are described in more detail below.

In some embodiments, the present invention provides a form of compound A free base substantially free of impurities. As used herein, the term “substantially free of impurities” means that the compound contains no significant amount of extraneous matter. Such extraneous matter may include different forms of compound A free base, residual solvents, or any other impurities that may result from the preparation of, and/or isolation of, compound A. In certain embodiments, at least about 95% by weight of a form of compound A free base is present. In still other embodiments of the invention, at least about 99% by weight of a form of compound A free base is present.

According to one embodiment, a form of compound A free base is present in an amount of at least about 97, 97.5, 98.0, 98.5, 99, 99.5, 99.8 weight percent where the percentages are based on the total weight of the composition. According to another embodiment, a form of compound A free base contains no more than about 3.0 area percent HPLC of total organic impurities and, in certain embodiments, no more than about 1.5 area percent HPLC total organic impurities relative to the total area of the HPLC chromatogram. In other embodiments, a form of compound A free base contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for a form of compound A free base is also meant to include all tautomeric forms of compound A free base. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

It has been found that compound A free base can exist in a variety of solid forms. Exemplary such forms include polymorphs such as those described herein.

As used herein, the term “polymorph” refers to the different crystal structures into which a compound, or a salt or solvate thereof, can crystallize.

As used herein, the term “about”, when used in reference to a degree 2-theta value refers to the stated value ±0.2 degree 2-theta.

In certain embodiments, compound A free base is a crystalline solid. In other embodiments, compound A free base is a crystalline solid substantially free of amorphous compound A free base. As used herein, the term “substantially free of amorphous compound A free base” means that the compound contains no significant amount of amorphous compound A free base. In certain embodiments, at least about 95% by weight of crystalline compound A free base is present. In still other embodiments of the invention, at least about 99% by weight of crystalline compound A free base is present.

It has been found that compound A free base can exist in at least three distinct polymorphic forms. In certain embodiments, the present invention provides a polymorphic form of compound A free base referred to herein as Form A. In certain embodiments, the present invention provides a polymorphic form of compound A free base referred to herein as Form B. In certain embodiments, the present invention provides a polymorphic form of compound A free base referred to herein as Form C.

In some embodiments, compound A free base is amorphous. In some embodiments, compound A free base is amorphous, and is substantially free of crystalline compound A free base.

### Form A of Compound A

In some embodiments, Form A of compound A free base has at least 1, 2, 3, 4 or 5 spectral peak(s) selected from the peaks listed in Table 1 below.

TABLE 1
XRPD Peak Positions for Form A of Compound A Free Base

| Position (°2θ)¹ | Intensity cps° |
|---|---|
| 9.0 | 1057 |
| 11.5 | 2282 |
| 12.9 | 1758 |
| 16.8 | 1433 |
| 17.9 | 1752 |
| 21.2 | 1097 |
| 22.0 | 443 |
| 23.5 | 1823 |
| 25.7 | 936 |
| 28.2 | 1093 |

¹In this and all subsequent tables, the position 2θ is within ±0.2.

In some embodiments, Form A of compound A is characterized in that it has one or more peaks in its X-ray powder diffraction (XRPD) pattern selected from those at about 11.5, about 12.9 and about 23.5 degrees 2-theta. In some embodiments, Form A of compound A free base is characterized in that it has two or more peaks in its X-ray powder diffraction pattern selected from those at about 11.5, about 12.9 and about 23.5 degrees 2-theta. In some embodiments, Form A of compound A free base is characterized in that it has all three peaks in its X-ray powder diffraction pattern selected from those at about 11.5, about 12.9 and about 23.5 degrees 2-theta.

As used herein, the term “about”, when used in reference to a degree 2-theta value refers to the stated value ±0.2 degree 2-theta.

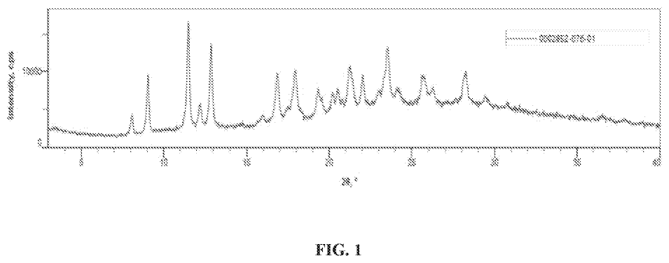

In certain embodiments, the X-ray powder diffraction pattern is substantially similar to the XRPD provided in FIG. 1.

Methods for preparing Form A of compound A free base are described infra.

### Form B of Compound A

In some embodiments, Form B of compound A free base has at least 1, 2, 3, 4 or 5 spectral peak(s) selected from the peaks listed in Table 2 below.

TABLE 2
XRPD Peak Positions for Form B of Compound A Free Base

| Position (°2θ) | Intensity cps° |
|---|---|
| 11.2 | 1838 |
| 16.0 | 1234 |
| 17.8 | 2637 |
| 17.9 | 634 |
| 18.3 | 3016 |
| 19.1 | 640 |
| 20.8 | 5682 |
| 22.5 | 874 |
| 22.9 | 1368 |
| 31.6 | 1095 |

In some embodiments, Form B of compound A free base is characterized in that it has one or more peaks in its X-ray powder diffraction (XRPD) pattern selected from those at about 17.8, about 18.3 and about 20.8 degrees 2-theta. In some embodiments, Form B of compound A free base is characterized in that it has two or more peaks in its X-ray powder diffraction pattern selected from those at about 17.8, about 18.3 and about 20.8 degrees 2-theta. In some embodiments, Form B of compound A free base is characterized in that it has all three peaks in its X-ray powder diffraction pattern selected from those at about 17.8, about 18.3 and about 20.8 degrees 2-theta. As used herein, the term “about”, when used in reference to a degree 2-theta value refers to the stated value ±0.2 degree 2-theta.

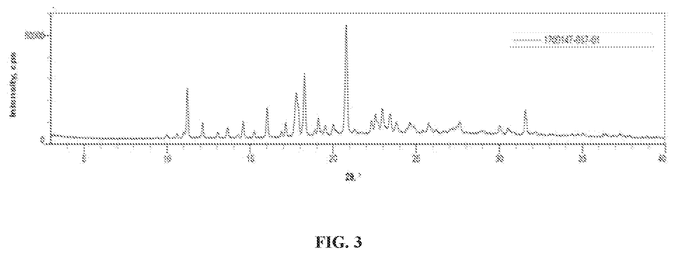

In certain embodiments, the X-ray powder diffraction pattern is substantially similar to the XRPD provided in FIG. 3.

Methods for preparing Form B of compound A free base are described infra.

### Form C of Compound A

In some embodiments, Form C of compound A free base has at least 1, 2, 3, 4 or 5 spectral peak(s) selected from the peaks listed in Table 3 below.

TABLE 3
XRPD Peak Positions for Form C of Compound A Free Base

| Position (°2θ) | Intensity cps° |
|---|---|
| 13.6 | 1965 |
| 16.4 | 2483 |
| 17.2 | 1233 |
| 20.0 | 1322 |
| 20.6 | 1123 |
| 20.8 | 1744 |
| 22.6 | 974 |
| 22.9 | 2889 |
| 23.7 | 1384 |
| 31.6 | 2668 |

In some embodiments, Form C of compound A free base is characterized in that it has one or more peaks in its X-ray powder diffraction (XRPD) pattern selected from those at about 16.4, about 22.9 and about 31.6 degrees 2-theta. In some embodiments, Form C of compound A free base is characterized in that it has two or more peaks in its X-ray powder diffraction pattern selected from those at about 16.4, about 22.9 and about 31.6 degrees 2-theta. In some embodiments, Form C of compound A free base is characterized in that it has all three peaks in its X-ray powder diffraction pattern selected from those at about 16.4, about 22.9 and about 31.6 degrees 2-theta. As used herein, the term “about”, when used in reference to a degree 2-theta value refers to the stated value ±0.2 degree 2-theta.

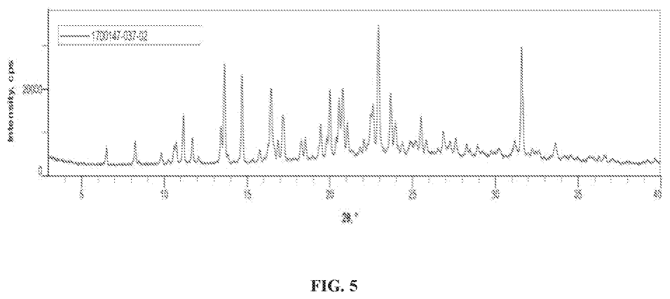

In certain embodiments, the X-ray powder diffraction pattern is substantially similar to the XRPD provided in FIG. 5.

Methods for preparing Form C of compound A free base are described infra.

In some embodiments, the present invention provides compound A free base:

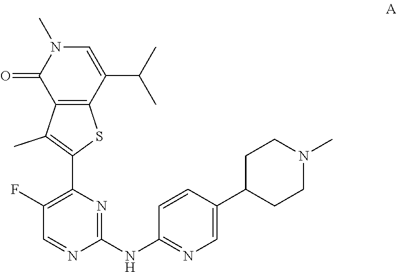 [SMILES: Cc1c(-c2nc(Nc3ccc(C4CCN(C)CC4)cn3)ncc2F)sc2c(C(C)C)cn(C)c(=O)c12]

wherein said compound is crystalline.

In some embodiments, the present invention provides compound A free base, wherein said compound is substantially free of amorphous compound A free base.

In some embodiments, the present invention provides compound A free base, wherein said compound is substantially free of impurities.

In some embodiments, the present invention provides compound A free base, wherein said compound has one or more peaks in its XRPD selected from those at about 11.5, about 12.9 and about 23.5 degrees 2-theta. In some such embodiments, the present invention provides compound A free base, wherein said compound has at least two peaks in its XRPD selected from those at about about 11.5, about 12.9 and about 23.5 degrees 2-theta. In some such embodiments, the present invention provides compound A succinate salt, wherein said compound has at least three peaks in its XRPD selected from those at about about 11.5, about 12.9 and about 23.5 degrees 2-theta. In some such embodiments, the present invention provides Compound A free base, wherein said compound is of Form A.

In some embodiments, the present invention provides compound A free base, wherein said compound has an XRPD substantially similar to that depicted in FIG. 1.

In some embodiments, the present invention provides compound A free base, wherein said compound has one or more peaks in its XRPD selected from those at about 17.8, about 18.3 and about 20.8 degrees 2-theta. In some such embodiments, the present invention provides compound A free base, wherein said compound has at least two peaks in its XRPD selected from those at about 17.8, about 18.3 and about 20.8 degrees 2-theta. In some such embodiments, the present invention provides compound A free base, wherein said compound has at least three peaks in its XRPD selected from those at about 17.8, about 18.3 and about 20.8 degrees 2-theta. In some such embodiments, the present invention provides compound A free base, wherein said compound is of Form B.

In some embodiments, the present invention provides compound A free base, wherein said compound has an XRPD substantially similar to that depicted in FIG. 3.

In some embodiments, the present invention provides compound A free base, wherein said compound has one or more peaks in its XRPD selected from those at about 16.4, about 22.9 and about 31.6 degrees 2-theta. In some such embodiments, the present invention provides compound A free base, wherein said compound has at least two peaks in its XRPD selected from those at about 16.4, about 22.9 and about 31.6 degrees 2-theta. In some such embodiments, the present invention provides compound A free base, wherein said compound has at least three peaks in its XRPD selected from those at about 16.4, about 22.9 and about 31.6 degrees 2-theta. In some such embodiments, the present invention provides compound A free base, wherein said compound is of Form C.

In some embodiments, the present invention provides compound A free base, wherein said compound has an XRPD substantially similar to that depicted in FIG. 5.

In some embodiments, the present invention provides a composition comprising compound A free base and a pharmaceutically acceptable carrier or excipient.

In some embodiments, the present invention provides a method of treating a CDK4-mediated and a CDK6-mediated disorder in a patient in need thereof, comprising administering to said patient compound A free base or composition thereof. In some embodiments, compound A free base is of Form A. In some embodiments, compound A free base is of Form B. In some embodiments, compound A free base is of Form C.

In some embodiments, the CDK4-mediated and CDK6-mediated disorder is a cancer. In some embodiments, the cancer is breast cancer, malignant brain tumors, colon cancer, small-cell lung cancer, non-small-cell lung cancer, bladder cancer, ovarian cancer, prostate cancer, chronic lymphoid leukemia, lymphoma, myeloma, acute myeloid leukemia, secondary pancreatic cancer or secondary brain metastases.

In some embodiments, the breast cancer is HR+/HER2− or HR+/HER2+ advanced or metastatic breast cancer; and the malignant brain tumors are glioblastoma, astrocytoma, or pontine glioma.

In some embodiments, the patient is administered a pharmaceutical composition of compound A free base. In some embodiments, the administration is oral administration of compound A free base. In some embodiments, the administration is intravenous administration of compound A free base.

In some embodiments, the methods described herein further comprise administering an additional therapeutic agent to the patient. In some embodiments, the additional therapeutic agent is a PRMT5 inhibitor, a HER2 kinase inhibitor, an aromatase inhibitor, an estrogen receptor antagonist or an alkylating agent. In some embodiments, the aromatase inhibitor is letrozole. In some embodiments, the estrogen receptor antagonist is fulvestrant. In some embodiments, the alkylating agent is temozolomide.

### Salt Forms of Compound A

In some embodiments, an acid and compound A are ionically bonded to form compound A succinate salt, described below. It is contemplated that compound A succinate salt can exist in a variety of physical forms. For example, compound A succinate salt can be in solution, suspension, or in solid form. In certain embodiments, compound A succinate salt is in solid form. When compound A succinate salt is in solid form, said compound may be amorphous, crystalline, or a mixture thereof. Exemplary such solid forms of compound A succinate salt are described in more detail below.

### Succinate Salts of Compound A

According to one embodiment, the present invention provides a succinate salt of compound A:

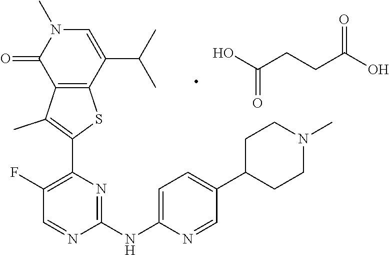

It will be appreciated by one of ordinary skill in the art that the succinic acid and compound A are ionically bonded to form compound A succinate salt. It is contemplated that compound A succinate salt can exist in a variety of physical forms. For example, compound A succinate salt can be in solution, suspension, or in solid form. In certain embodiments, compound A succinate salt is in solid form. When compound A succinate salt is in solid form, said compound may be amorphous, crystalline, or a mixture thereof. Exemplary solid forms are described in more detail below.

In some embodiments, the present invention provides compound A succinate salt substantially free of impurities. As used herein, the term “substantially free of impurities” means that the compound contains no significant amount of extraneous matter. Such extraneous matter may include excess succinic acid, excess compound A, residual solvents, or any other impurities that may result from the preparation of, and/or isolation of, compound A succinate salt. In certain embodiments, at least about 95% by weight of compound A succinate salt is present. In still other embodiments of the invention, at least about 99% by weight of compound A succinate salt is present.

According to one embodiment, compound A succinate salt is present in an amount of at least about 97, 97.5, 98.0, 98.5, 99, 99.5, 99.8 weight percent where the percentages are based on the total weight of the composition. According to another embodiment, compound A succinate salt contains no more than about 3.0 area percent HPLC of total organic impurities and, in certain embodiments, no more than about 1.5 area percent HPLC total organic impurities relative to the total area of the HPLC chromatogram. In other embodiments, compound A succinate salt contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for compound A succinate salt is also meant to include all tautomeric forms of compound A succinate salt. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

In certain embodiments, compound A succinate salt is a crystalline solid. In other embodiments, compound A succinate salt is a crystalline solid substantially free of amorphous compound A succinate salt. As used herein, the term “substantially free of amorphous compound A succinate salt” means that the compound contains no significant amount of amorphous compound A succinate salt. In certain embodiments, at least about 95% by weight of crystalline compound A succinate salt is present. In still other embodiments of the invention, at least about 99% by weight of crystalline compound A succinate salt is present.

It has been found that compound A succinate salt can exist in at least seven distinct polymorphic forms. In some embodiments, the present invention provides a polymorphic form of Compound A succinate salt referred to herein as Form A. In certain embodiments, the present invention provides a polymorphic form of compound A succinate salt referred to herein as Form B. In some embodiments, the present invention provides a polymorphic form of Compound A succinate salt referred to herein as Form C. In certain embodiments, the present invention provides a polymorphic form of compound A succinate salt referred to herein as Form D. In some embodiments, the present invention provides a polymorphic form of Compound A succinate salt referred to herein as Form E. In certain embodiments, the present invention provides a polymorphic form of compound A succinate salt referred to herein as Form F. In some embodiments, the present invention provides a polymorphic form of Compound A succinate salt referred to herein as Form G.

In some embodiments, compound A succinate salt is amorphous. In some embodiments, compound A succinate salt is amorphous, and is substantially free of crystalline compound A succinate salt.

### Form A of Compound A Succinate Salt

In some embodiments, Form A of compound A succinate salt has at least 1, 2, 3, 4 or 5 spectral peak(s) selected from the peaks listed in Table 4 below.

TABLE 4
XRPD Peak Positions for Form A of Compound A Succinate Salt

| Position (°2θ) | Intensity cps° |
|---|---|
| 8.1 | 1598 |
| 10.8 | 1007 |
| 11.0 | 741 |
| 11.6 | 3424 |
| 12.0 | 556 |
| 13.6 | 860 |
| 15.8 | 640 |
| 17.5 | 872 |
| 24.4 | 1148 |
| 25.2 | 1001 |

In some embodiments, Form A of compound A succinate salt is characterized in that it has one or more peaks in its X-ray powder diffraction pattern selected from those at about 8.1, about 11.6 and about 24.4 degrees 2-theta. In some embodiments, Form A of compound A succinate salt is characterized in that it has two or more peaks in its X-ray powder diffraction pattern selected from those at about 8.1, about 11.6 and about 24.4 degrees 2-theta. In some embodiments, Form A of compound A succinate salt is characterized in that it has all three peaks in its X-ray powder diffraction pattern selected from those at about 8.1, about 11.6 and about 24.4 degrees 2-theta.

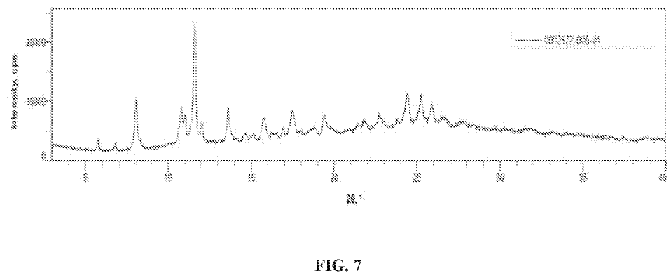

In certain embodiments, the X-ray powder diffraction pattern is substantially similar to the XRPD provided in FIG. 7.

Methods for preparing Form A of compound A succinate salt are described infra.

### Form B of Compound A Succinate Salt

In some embodiments, Form B of compound A succinate salt has at least 1, 2, 3, 4 or 5 spectral peak(s) selected from the peaks listed in Table 5 below.

TABLE 5
XRPD Peak Positions for Form B of Compound A Succinate Salt

| Position (°2θ) | Intensity cps° |
|---|---|
| 11.9 | 1476 |
| 12.3 | 1571 |
| 14.3 | 388 |
| 15.1 | 786 |
| 18.6 | 2087 |
| 20.6 | 411 |
| 21.8 | 629 |
| 24.1 | 1904 |
| 24.6 | 2561 |
| 25.6 | 715 |

In some embodiments, Form B of compound A succinate salt is characterized in that it has one or more peaks in its X-ray powder diffraction pattern selected from those at about 18.6, about 24.1 and about 24.6 degrees 2-theta. In some embodiments, Form B of compound A succinate salt is characterized in that it has two or more peaks in its X-ray powder diffraction pattern selected from those at about 18.6, about 24.1 and about 24.6 degrees 2-theta. In some embodiments, Form B of compound A succinate salt is characterized in that it has all three peaks in its X-ray powder diffraction pattern selected from those at about 18.6, about 24.1 and about 24.6 degrees 2-theta.

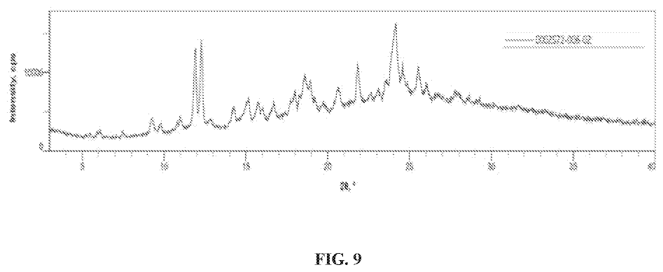

In certain embodiments, the X-ray powder diffraction pattern is substantially similar to the XRPD provided in FIG. 9.

Methods for preparing Form B of compound A succinate salt are described infra.

### Form C of Compound A Succinate Salt

In some embodiments, Form C of compound A succinate salt has at least 1, 2, 3, 4 or 5 spectral peak(s) selected from the peaks listed in Table 6 below.

TABLE 6
XRPD Peak Positions for Form C of Compound A Succinate Salt

| Position (°2θ) | Intensity cps° |
|---|---|
| 9.2 | 3483 |
| 11.0 | 1956 |
| 11.9 | 3746 |
| 14.2 | 2881 |
| 15.1 | 3132 |
| 18.6 | 3295 |
| 18.9 | 3877 |
| 23.9 | 1819 |
| 24.0 | 2111 |
| 24.2 | 3363 |

In some embodiments, Form C of compound A succinate salt is characterized in that it has one or more peaks in its X-ray powder diffraction pattern selected from those at about 9.2, about 11.9 and about 18.9 degrees 2-theta. In some embodiments, Form C of compound A succinate salt is characterized in that it has two or more peaks in its X-ray powder diffraction pattern selected from those at about 9.2, about 11.9 and about 18.9 and about 18.9 degrees 2-theta. In some embodiments, Form C of compound A succinate salt is characterized in that it has all three peaks in its X-ray powder diffraction pattern selected from those at about 9.2, about 11.9 and about 18.9 degrees 2-theta.

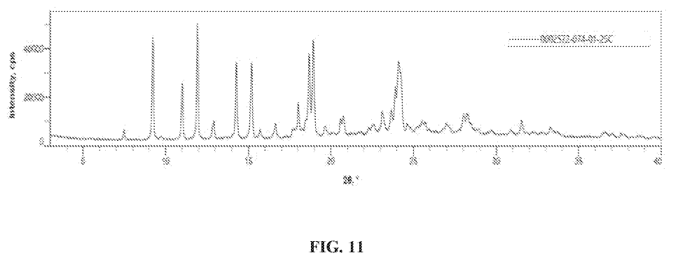

In certain embodiments, the X-ray powder diffraction pattern is substantially similar to the XRPD provided in FIG. 11.

Methods for preparing Form C of compound A succinate salt are described infra.

### Form D of Compound A Succinate Salt

In some embodiments, Form D of compound A succinate salt has at least 1, 2, 3, 4 or 5 spectral peak(s) selected from the peaks listed in Table 7 below.

TABLE 7
XRPD Peak Positions for Form D of Compound A Succinate Salt

| Position (°2θ) | Intensity cps° |
|---|---|
| 11.3 | 1074 |
| 12.3 | 2971 |
| 15.2 | 378 |
| 18.5 | 1582 |
| 19.4 | 2426 |
| 21.0 | 692 |
| 22.7 | 848 |
| 24.3 | 2178 |
| 24.7 | 2637 |
| 25.4 | 973 |

In some embodiments, Form D of compound A succinate salt is characterized in that it has one or more peaks in its X-ray powder diffraction pattern selected from those at about 12.3, about 19.4 and about 24.7 degrees 2-theta. In some embodiments, Form D of compound A succinate salt is characterized in that it has two or more peaks in its X-ray powder diffraction pattern selected from those at about 12.3, about 19.4 and about 24.7 and about 18.9 degrees 2-theta. In some embodiments, Form D of compound A succinate salt is characterized in that it has all three peaks in its X-ray powder diffraction pattern selected from those at about 12.3, about 19.4 and about 24.7 degrees 2-theta.

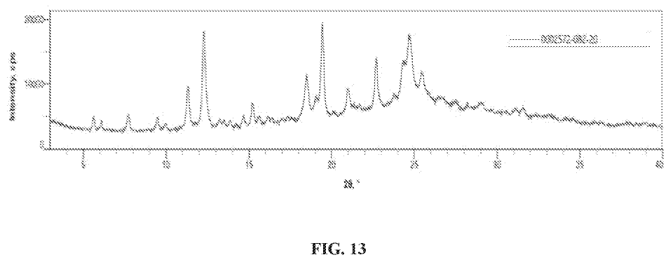

In certain embodiments, the X-ray powder diffraction pattern is substantially similar to the XRPD provided in FIG. 13.

Methods for preparing Form D of compound A succinate salt are described infra.

### Form E of Compound A Succinate Salt

In some embodiments, Form E of compound A succinate salt has at least 1, 2, 3, 4 or 5 spectral peak(s) selected from the peaks listed in Table 8 below.

TABLE 8
XRPD Peak Positions for Form E of Compound A Succinate Salt

| Position (°2θ) | Intensity cps° |
|---|---|
| 9.2 | 773 |
| 11.0 | 285 |
| 11.9 | 2806 |
| 14.2 | 652 |
| 15.1 | 1170 |
| 18.0 | 1309 |
| 18.6 | 993 |
| 18.9 | 1059 |
| 23.1 | 1781 |
| 24.1 | 2603 |

In some embodiments, Form E of compound A succinate salt is characterized in that it has one or more peaks in its X-ray powder diffraction pattern selected from those at about 11.9, about 23.1 and about 24.1 degrees 2-theta. In some embodiments, Form E of compound A succinate salt is characterized in that it has two or more peaks in its X-ray powder diffraction pattern selected from those at about 11.9, about 23.1 and about 24.1 and about 18.9 degrees 2-theta. In some embodiments, Form E of compound A succinate salt is characterized in that it has all three peaks in its X-ray powder diffraction pattern selected from those at about 11.9, about 23.1 and about 24.1 degrees 2-theta.

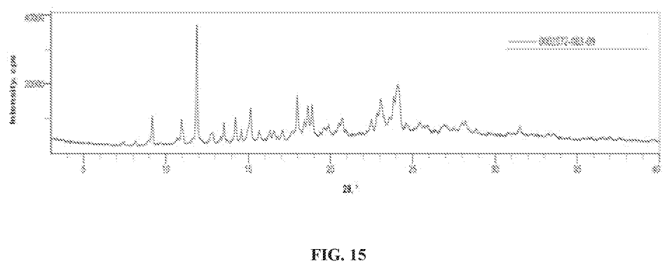

In certain embodiments, the X-ray powder diffraction pattern is substantially similar to the XRPD provided in FIG. 15.

Methods for preparing Form E of compound A succinate salt are described infra.

### Form F of Compound A Succinate Salt

In some embodiments, Form F of compound A succinate salt has at least 1, 2, 3, 4 or 5 spectral peak(s) selected from the peaks listed in Table 9 below.

TABLE 9
XRPD Peak Positions for Form F of Compound A Succinate Salt

| Position (°2θ) | Rel. Int. [%] |
|---|---|
| 6.0 | 69.55 |
| 6.5 | 34.06 |
| 8.1 | 11.08 |
| 9.8 | 19.53 |
| 11.6 | 18.09 |
| 12.5 | 100.00 |
| 13.5 | 9.30 |
| 14.8 | 10.73 |
| 15.8 | 40.61 |
| 16.4 | 16.76 |
| 17.2 | 18.19 |
| 18.4 | 40.48 |
| 18.6 | 37.40 |
| 19.1 | 31.21 |
| 20.2 | 9.75 |
| 21.2 | 36.05 |
| 22.4 | 7.29 |
| 23.7 | 17.44 |
| 24.5 | 90.59 |
| 24.7 | 90.50 |
| 26.3 | 15.31 |
| 27.4 | 10.51 |
| 28.5 | 12.96 |
| 32.1 | 3.11 |
| 34.0 | 3.97 |
| 37.1 | 3.14 |

In some embodiments, Form F of compound A succinate salt is characterized in that it has one or more peaks in its X-ray powder diffraction pattern selected from those at about 6.0, about 12.5 and about 24.5 degrees 2-theta. In some embodiments, Form F of compound A succinate salt is characterized in that it has two or more peaks in its X-ray powder diffraction pattern selected from those at about 6.0, about 12.5 and about 24.5 and about 18.9 degrees 2-theta. In some embodiments, Form F of compound A succinate salt is characterized in that it has all three peaks in its X-ray powder diffraction pattern selected from those at about 6.0, about 12.5 and about 24.5 degrees 2-theta.

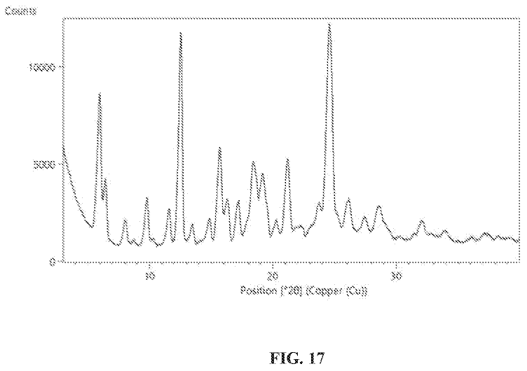

In certain embodiments, the X-ray powder diffraction pattern is substantially similar to the XRPD provided in FIG. 17.

Methods for preparing Form F of compound A succinate salt are described infra.

### Form G of Compound A Succinate Salt

In some embodiments, Form G of compound A succinate salt has at least 1, 2, 3, 4 or 5 spectral peak(s) selected from the peaks listed in Table 10 below.

TABLE 10
XRPD Peak Positions for Form G of Compound A Succinate Salt

| Position (°2θ) | Rel. Int. [%] |
|---|---|
| 4.6 | 3.48 |
| 5.4 | 32.18 |
| 6.0 | 100.00 |
| 7.5 | 32.37 |
| 9.1 | 0.46 |
| 9.6 | 0.72 |
| 10.0 | 3.90 |
| 10.9 | 5.72 |
| 11.9 | 19.84 |
| 12.8 | 14.01 |
| 14.0 | 7.12 |
| 14.2 | 3.37 |
| 15.1 | 35.06 |
| 15.3 | 37.67 |
| 15.6 | 3.32 |
| 15.9 | 2.56 |
| 16.4 | 3.81 |
| 16.9 | 3.49 |
| 17.2 | 15.36 |
| 17.8 | 10.88 |
| 17.9 | 23.54 |
| 18.3 | 3.34 |
| 18.6 | 5.86 |
| 18.9 | 9.48 |
| 19.3 | 10.66 |
| 19.8 | 7.14 |
| 20.0 | 16.43 |
| 20.4 | 5.45 |
| 20.8 | 4.20 |
| 21.0 | 2.46 |
| 21.5 | 1.70 |
| 22.0 | 8.62 |
| 22.3 | 1.26 |
| 22.9 | 9.44 |
| 23.1 | 21.49 |
| 23.3 | 26.91 |
| 23.4 | 36.38 |
| 23.5 | 21.20 |
| 24.0 | 15.44 |
| 24.2 | 10.39 |
| 24.4 | 3.00 |
| 24.6 | 5.88 |
| 24.8 | 3.52 |
| 25.1 | 15.50 |
| 25.7 | 1.04 |
| 26.5 | 1.35 |
| 27.2 | 8.67 |
| 27.7 | 22.08 |
| 28.0 | 2.65 |
| 28.4 | 5.03 |
| 28.6 | 2.23 |
| 29.1 | 4.45 |
| 29.5 | 2.31 |
| 29.9 | 0.78 |
| 30.1 | 1.88 |
| 30.8 | 1.77 |
| 31.2 | 2.08 |
| 31.7 | 2.20 |
| 32.1 | 1.43 |
| 32.4 | 2.60 |
| 32.5 | 3.02 |
| 32.7 | 1.29 |
| 32.9 | 0.95 |
| 33.2 | 3.46 |
| 33.5 | 2.15 |
| 34.3 | 3.46 |
| 34.5 | 1.88 |
| 34.9 | 2.81 |
| 35.2 | 1.27 |
| 35.7 | 3.28 |
| 36.2 | 0.86 |
| 36.7 | 1.54 |
| 37.6 | 2.38 |
| 37.9 | 0.89 |
| 38.3 | 1.25 |
| 38.5 | 1.60 |
| 39.4 | 1.18 |

In some embodiments, Form G of compound A succinate salt is characterized in that it has one or more peaks in its X-ray powder diffraction pattern selected from those at about 6.0, about 15.3 and about 23.4 degrees 2-theta. In some embodiments, Form G of compound A succinate salt is characterized in that it has two or more peaks in its X-ray powder diffraction pattern selected from those at about 6.0, about 15.3 and about 23.4 and about 18.9 degrees 2-theta. In some embodiments, Form G of compound A succinate salt is characterized in that it has all three peaks in its X-ray powder diffraction pattern selected from those at about 6.0, about 15.3 and about 23.4 degrees 2-theta.

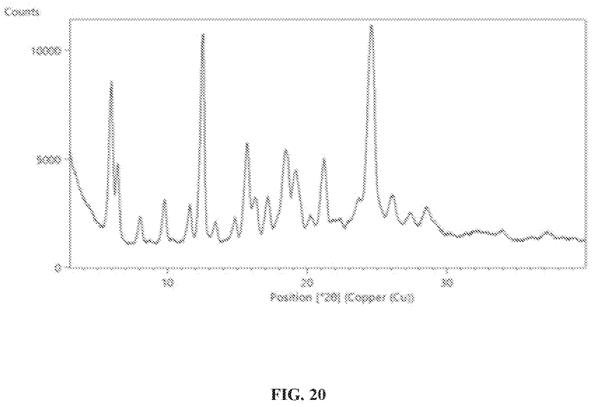

In certain embodiments, the X-ray powder diffraction pattern is substantially similar to the XRPD provided in FIG. 20.

Methods for preparing Form G of compound A succinate salt are described infra.

In some embodiments, the present invention provides compound A succinate salt:

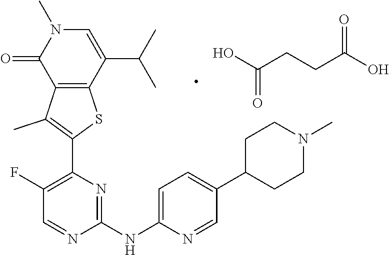

In some embodiments, the present invention provides compound A succinate salt, wherein said compound is crystalline.

In some embodiments, the present invention provides compound A succinate salt, wherein said compound is a crystalline solid substantially free of amorphous compound A succinate salt.

In some embodiments, the present invention provides compound A succinate salt, wherein said compound is substantially free of impurities.

In some embodiments, the present invention provides compound A succinate salt, wherein said compound has one or more peaks in its XRPD selected from those at about 8.1, about 11.6 and about 24.4 degrees 2-theta. In some such embodiments, the present invention provides compound A succinate salt, wherein said compound has at least two peaks in its XRPD selected from those at about 8.1, about 11.6 and about 24.4 degrees 2-theta. In some such embodiments, the present invention provides compound A succinate salt, wherein said compound has at least three peaks in its XRPD selected from those at about 8.1, about 11.6 and about 24.4 degrees 2-theta. In some such embodiments, the present invention provides compound A succinate salt, wherein said compound is of Form A.

In some embodiments, the present invention provides compound A succinate salt, wherein said compound has an XRPD substantially similar to that depicted in FIG. 7.

In some embodiments, the present invention provides compound A succinate salt, wherein said compound has one or more peaks in its XRPD selected from those at about 18.6, about 24.1 and about 24.6 degrees 2-theta. In some such embodiments, the present invention provides compound A succinate salt, wherein said compound has at least two peaks in its XRPD selected from those at about 18.6, about 24.1 and about 24.6 degrees 2-theta. In some such embodiments, the present invention provides compound A succinate salt, wherein said compound has at least three peaks in its XRPD selected from those at about 18.6, about 24.1 and about 24.6 degrees 2-theta. In some such embodiments, the present invention provides compound A succinate salt, wherein said compound is of Form B.

In some embodiments, the present invention provides compound A succinate salt, wherein said compound has an XRPD substantially similar to that depicted in FIG. 9.

In some embodiments, the present invention provides compound A succinate salt, wherein said compound has one or more peaks in its XRPD selected from those at about 9.2, about 11.9 and about 18.9 degrees 2-theta. In some such embodiments, the present invention provides compound A succinate salt, wherein said compound has at least two peaks in its XRPD selected from those at about 9.2, about 11.9 and about 18.9 degrees 2-theta. In some such embodiments, the present invention provides compound A succinate salt, wherein said compound has at least three peaks in its XRPD selected from those at about 9.2, about 11.9 and about 18.9 degrees 2-theta. In some such embodiments, the present invention provides compound A succinate salt, wherein said compound is of Form C.

In some embodiments, the present invention provides compound A succinate salt, wherein said compound has an XRPD substantially similar to that depicted in FIG. 11.

In some embodiments, the present invention provides compound A succinate salt, wherein said compound has one or more peaks in its XRPD selected from those at about 12.3, about 19.4 and about 24.7 degrees 2-theta. In some such embodiments, the present invention provides compound A succinate salt, wherein said compound has at least two peaks in its XRPD selected from those at about 12.3, about 19.4 and about 24.7 degrees 2-theta. In some such embodiments, the present invention provides compound A succinate salt, wherein said compound has at least three peaks in its XRPD selected from those at about 12.3, about 19.4 and about 24.7 degrees 2-theta. In some such embodiments, the present invention provides compound A succinate salt, wherein said compound is of Form D.

In some embodiments, the present invention provides compound A succinate salt, wherein said compound has an XRPD substantially similar to that depicted in FIG. 13.

In some embodiments, the present invention provides compound A succinate salt, wherein said compound has one or more peaks in its XRPD selected from those at about 11.9, about 23.1 and about 24.1 degrees 2-theta. In some such embodiments, the present invention provides compound A succinate salt, wherein said compound has at least two peaks in its XRPD selected from those at about 11.9, about 23.1 and about 24.1 degrees 2-theta. In some such embodiments, the present invention provides compound A succinate salt, wherein said compound has at least three peaks in its XRPD selected from those at about 11.9, about 23.1 and about 24.1 degrees 2-theta. In some such embodiments, the present invention provides compound A succinate salt, wherein said compound is of Form E.

In some embodiments, the present invention provides compound A succinate salt, wherein said compound has an XRPD substantially similar to that depicted in FIG. 15.

In some embodiments, the present invention provides compound A succinate salt, wherein said compound has one or more peaks in its XRPD selected from those at about 6.0, about 12.5 and about 24.5 degrees 2-theta. In some such embodiments, the present invention provides compound A succinate salt, wherein said compound has at least two peaks in its XRPD selected from those at about 6.0, about 12.5 and about 24.5 degrees 2-theta. In some such embodiments, the present invention provides compound A succinate salt, wherein said compound has at least three peaks in its XRPD selected from those at about 6.0, about 12.5 and about 24.5 degrees 2-theta. In some such embodiments, the present invention provides compound A succinate salt, wherein said compound is of Form F.

In some embodiments, the present invention provides compound A succinate salt, wherein said compound has an XRPD substantially similar to that depicted in FIG. 17.

In some embodiments, the present invention provides compound A succinate salt, wherein said compound has one or more peaks in its XRPD selected from those at about 6.0, about 15.3 and about 23.4 degrees 2-theta. In some such embodiments, the present invention provides compound A succinate salt, wherein said compound has at least two peaks in its XRPD selected from those at about 6.0, about 15.3 and about 23.4 degrees 2-theta. In some such embodiments, the present invention provides compound A succinate salt, wherein said compound has at least three peaks in its XRPD selected from those at about 6.0, about 15.3 and about 23.4 degrees 2-theta. In some such embodiments, the present invention provides compound A succinate salt, wherein said compound is of Form G.

In some embodiments, the present invention provides compound A succinate salt, wherein said compound has an XRPD substantially similar to that depicted in FIG. 20.

In some embodiments, the present invention provides a composition comprising compound A succinate salt and a pharmaceutically acceptable carrier or excipient.

In some embodiments, the present invention provides a method of treating a CDK4-mediated and a CDK6-mediated disorder in a patient in need thereof, comprising administering to said patient compound A succinate salt or composition thereof. In some embodiments, compound A succinate salt is of Form A. In some embodiments, compound A succinate salt is of Form B. In some embodiments, compound A succinate salt is of Form C. In some embodiments, compound A succinate salt is of Form D. In some embodiments, compound A succinate salt is of Form E. In some embodiments, compound A succinate salt is of Form F. In some embodiments, compound A succinate salt is of Form G.

In some embodiments, the CDK4-mediated and CDK6-mediated disorder is a cancer. In some embodiments, the cancer is breast cancer, malignant brain tumors, colon cancer, small-cell lung cancer, non-small-cell lung cancer, bladder cancer, ovarian cancer, prostate cancer, chronic lymphoid leukemia, lymphoma, myeloma, acute myeloid leukemia, secondary pancreatic cancer or secondary brain metastases.

In some embodiments, the breast cancer is HR+/HER2− or HR+/HER2+ advanced or metastatic breast cancer; and the malignant brain tumors are glioblastoma, astrocytoma, or pontine glioma.

In some embodiments, the patient is administered a pharmaceutical composition of compound A succinate salt. In some embodiments, the administration is oral administration of compound A succinate salt. In some embodiments, the administration is intravenous administration of compound A succinate salt.

In some embodiments, the methods described herein further comprise administering an additional therapeutic agent to the patient. In some embodiments, the additional therapeutic agent is a PRMT5 inhibitor, a HER2 kinase inhibitor, an aromatase inhibitor, an estrogen receptor antagonist or an alkylating agent. In some embodiments, the aromatase inhibitor is letrozole. In some embodiments, the estrogen receptor antagonist is fulvestrant. In some embodiments, the alkylating agent is temozolomide.

### Pharmaceutical Salts of Compound A

In some embodiments, the disclosure is directed to a pharmaceutical salt of Compound A:

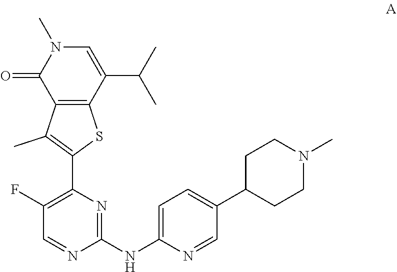 [SMILES: Cc1c(-c2nc(Nc3ccc(C4CCN(C)CC4)cn3)ncc2F)sc2c(C(C)C)cn(C)c(=O)c12]

wherein the salt is a hydrochloride, sulfate, maleate, phosphate, L-tartarate, fumarate, citrate, L-malate, tosylate, succinate, methanesulfonate, ethanesulfonate, glutarate, n-acetylglycine, acetate, malonate, or sebacate salt.

In some embodiments, the disclosure is directed to a pharmaceutical salt of Compound A that is a hydrochloride salt. In some embodiments, the pharmaceutical salt of Compound A is a hydrochloride salt in amorphous form. In some embodiments, the pharmaceutical salt of Compound A is a hydrochloride salt in crystalline form.

In some embodiments, the disclosure is directed to a pharmaceutical salt of Compound A that is a sulfate salt. In some embodiments, the pharmaceutical salt of Compound A is a sulfate salt in amorphous form. In some embodiments, the pharmaceutical salt of Compound A is a sulfate salt in crystalline form.

In some embodiments, the disclosure is directed to a pharmaceutical salt of Compound A that is a maleate salt. In some embodiments, the pharmaceutical salt of Compound A is a maleate salt in amorphous form. In some embodiments, the pharmaceutical salt of Compound A is a maleate salt in crystalline form.

In some embodiments, the disclosure is directed to a pharmaceutical salt of Compound A that is a phosphate salt. In some embodiments, the pharmaceutical salt of Compound A is a phosphate salt in amorphous form. In some embodiments, the pharmaceutical salt of Compound A is a phosphate salt in crystalline form.

In some embodiments, the disclosure is directed to a pharmaceutical salt of Compound A that is an L-tartrate salt. In some embodiments, the pharmaceutical salt of Compound A is an L-tartrate salt in amorphous form. In some embodiments, the pharmaceutical salt of Compound A is an L-tartrate salt in crystalline form.

In some embodiments, the disclosure is directed to a pharmaceutical salt of Compound A that is a fumarate salt. In some embodiments, the pharmaceutical salt of Compound A is a fumarate salt in amorphous form. In some embodiments, the pharmaceutical salt of Compound A is a fumarate salt in crystalline form.

In some embodiments, the disclosure is directed to a pharmaceutical salt of Compound A that is a citrate salt. In some embodiments, the pharmaceutical salt of Compound A is a citrate salt in amorphous form. In some embodiments, the pharmaceutical salt of Compound A is a citrate salt in crystalline form.

In some embodiments, the disclosure is directed to a pharmaceutical salt of Compound A that is an L-malate salt. In some embodiments, the pharmaceutical salt of Compound A is an L-malate salt in amorphous form. In some embodiments, the pharmaceutical salt of Compound A is an L-malate salt in crystalline form.

In some embodiments, the disclosure is directed to a pharmaceutical salt of Compound A that is a tosylate salt. In some embodiments, the pharmaceutical salt of Compound A is a tosylate salt in amorphous form. In some embodiments, the pharmaceutical salt of Compound A is a tosylate salt in crystalline form.

In some embodiments, the disclosure is directed to a pharmaceutical salt of Compound A that is a succinate salt. In some embodiments, the pharmaceutical salt of Compound A is a succinate salt in amorphous form. In some embodiments, the pharmaceutical salt of Compound A is a succinate salt in crystalline form.

In some embodiments, the disclosure is directed to a pharmaceutical salt of Compound A that is a mesylate salt. In some embodiments, the pharmaceutical salt of Compound A is a mesylate salt in amorphous form. In some embodiments, the pharmaceutical salt of Compound A is a mesylate salt in crystalline form.

In some embodiments, the disclosure is directed to a pharmaceutical salt of Compound A that is an esylate salt. In some embodiments, the pharmaceutical salt of Compound A is an esylate salt in amorphous form. In some embodiments, the pharmaceutical salt of Compound A is an esylate salt in crystalline form.

In some embodiments, the disclosure is directed to a pharmaceutical salt of Compound A that is a glutarate salt. In some embodiments, the pharmaceutical salt of Compound A is a glutarate salt in amorphous form. In some embodiments, the pharmaceutical salt of Compound A is a glutarate salt in crystalline form.

In some embodiments, the disclosure is directed to a pharmaceutical salt of Compound A that is an aceturate salt. In some embodiments, the pharmaceutical salt of Compound A is an aceturate salt in amorphous form. In some embodiments, the pharmaceutical salt of Compound A is an aceturate salt in crystalline form.

In some embodiments, the disclosure is directed to a pharmaceutical salt of Compound A that is an acetate salt. In some embodiments, the pharmaceutical salt of Compound A is an acetate salt in amorphous form. In some embodiments, the pharmaceutical salt of Compound A is an acetate salt in crystalline form.

In some embodiments, the disclosure is directed to a pharmaceutical salt of Compound A that is a malonate salt. In some embodiments, the pharmaceutical salt of Compound A is a malonate salt in amorphous form. In some embodiments, the pharmaceutical salt of Compound A is a malonate salt in crystalline form.

In some embodiments, the disclosure is directed to a pharmaceutical salt of Compound A that is a sebacate salt. In some embodiments, the pharmaceutical salt of Compound A is a sebacate salt in amorphous form. In some embodiments, the pharmaceutical salt of Compound A is a sebacate salt in crystalline form.

### General Methods of Providing a Salt Compound

Compound A is prepared according to the methods described in detail in the '323 application, the entirety of which is hereby incorporated herein by reference. Salt compounds of general formula X, which formula encompasses, inter alia, salt compound A succinate salt and/or particular forms thereof, are prepared from compound A, according to the general Scheme below.

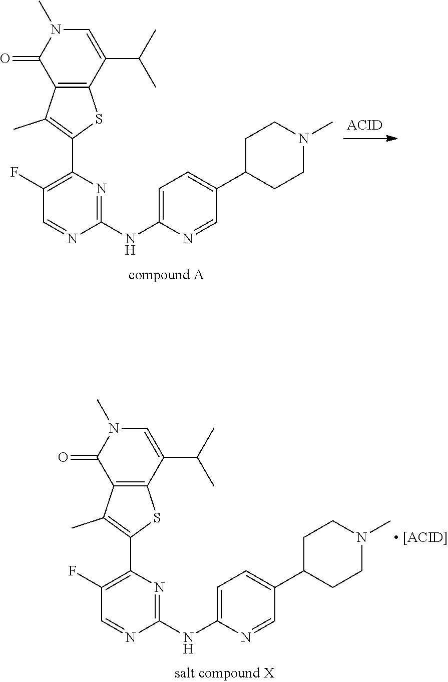

For instance, compound A succinate salt and forms thereof, are prepared from compound A by combining compound A with an appropriate acid to form a salt of that acid. Thus, another aspect of the present invention provides a method for preparing compound A succinate salt and forms thereof.

As described generally above, in some embodiments, the present invention provides a method for preparing a salt compound of the general formula X:

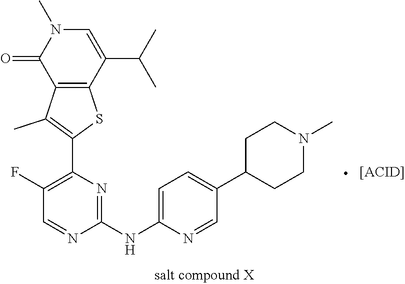

comprising steps of:
combining compound A:

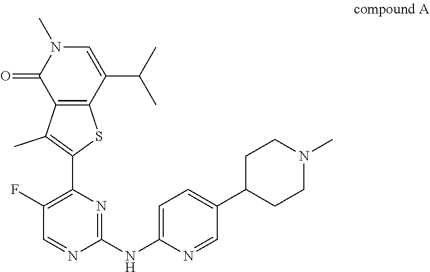 [SMILES: Cc1c(-c2nc(Nc3ccc(C4CCN(C)CC4)cn3)ncc2F)sc2c(C(C)C)cn(C)c(=O)c12]

with a suitable acid and optionally a suitable solvent under conditions suitable for forming a salt compound of general formula X.

In some embodiments, a suitable acid is succinic acid. In some embodiments, the present invention provides a method of making a succinate salt of compound A. In certain embodiments, the succinate salt of compound A is compound A succinate salt. In certain embodiments, the succinate salt of compound A is Form A of compound A succinate salt. In certain embodiments, the succinate salt of compound A is Form B of compound A succinate salt. In certain embodiments, the succinate salt of compound A is Form C of compound A succinate salt. In certain embodiments, the succinate salt of compound A is Form D of compound A succinate salt. In certain embodiments, the succinate salt of compound A is Form E of compound A succinate salt. In certain embodiments, the succinate salt of compound A is Form F of compound A succinate salt. In certain embodiments, the succinate salt of compound A is Form G of compound A succinate salt.

In some embodiments, a suitable acid is hydrochloric acid. In some embodiments, the present invention provides a method of making a hydrochloride salt of compound A. In certain embodiments, the hydrochloride salt of compound A is compound A hydrochloride salt. In certain embodiments, the hydrochloride salt of compound A is Form A of compound A hydrochloride salt. In certain embodiments, the hydrochloride salt of compound A is Form B of compound A hydrochloride salt. In certain embodiments, the hydrochloride salt of compound A is Form C of compound A hydrochloride salt. In certain embodiments, the hydrochloride salt of compound A is Form D of compound A hydrochloride salt. In certain embodiments, the hydrochloride salt of compound A is Form E of compound A hydrochloride salt. In certain embodiments, the hydrochloride salt of compound A is Form F of compound A hydrochloride salt.

In some embodiments, a suitable acid is maleic acid. In some embodiments, the present invention provides a method of making a malonate salt of compound A. In certain embodiments, the malonate salt of compound A is compound A malonate salt. In certain embodiments, the malonate salt of compound A is Form A of compound A malonate salt.

In some embodiments, a suitable acid is sulfuric acid. In some embodiments, the present invention provides a method of making a sulfate salt of compound A. In certain embodiments, the sulfate salt of compound A is compound A sulfate salt. In certain embodiments, the sulfate salt of compound A is Form A of compound A sulfate salt. In certain embodiments, the sulfate salt of compound A is Form B of compound A sulfate salt. In certain embodiments, the sulfate salt of compound A is Form C of compound A sulfate salt.

In some embodiments, a suitable acid is phosphoric acid. In some embodiments, the present invention provides a method of making a phosphate salt of compound A. In certain embodiments, the phosphate salt of compound A is compound A phosphate salt. In certain embodiments, the phosphate salt of compound A is Form A of compound A phosphate salt.

In some embodiments, a suitable acid is L-tartaric acid. In some embodiments, the present invention provides a method of making an L-tartrate salt of compound A. In certain embodiments, the phosphate salt of compound A is compound A L-tartrate salt. In certain embodiments, the L-tartrate salt of compound A is Form A of compound A L-tartrate salt.

In some embodiments, a suitable acid is fumaric acid. In some embodiments, the present invention provides a method of making a fumarate salt of compound A. In certain embodiments, the fumarate salt of compound A is compound A fumarate salt. In certain embodiments, the fumarate salt of compound A is Form A of compound A fumarate salt. In certain embodiments, the fumarate salt of compound A is Form B of compound A fumarate salt. In certain embodiments, the fumarate salt of compound A is Form C of compound A fumarate salt. In certain embodiments, the fumarate salt of compound A is Form D of compound A fumarate salt.

In some embodiments, a suitable acid is citric acid. In some embodiments, the present invention provides a method of making a citrate salt of compound A. In certain embodiments, the phosphate salt of compound A is compound A citrate salt. In certain embodiments, the citrate salt of compound A is Form A of compound A citrate salt.

In some embodiments, a suitable acid is L-malic acid. In some embodiments, the present invention provides a method of making an L-malate salt of compound A. In certain embodiments, the phosphate salt of compound A is compound A L-malate salt. In certain embodiments, the L-malate salt of compound A is Form A of compound A L-malate salt.

In some embodiments, a suitable acid is methanesulfonic acid. In some embodiments, the present invention provides a method of making a mesylate salt of compound A. In certain embodiments, the mesylate salt of compound A is compound A mesylate salt. In certain embodiments, the mesylate salt of compound A is Form A of compound A mesylate salt. In certain embodiments, the mesylate salt of compound A is Form B of compound A mesylate salt.

In some embodiments, a suitable acid is ethanesulfonic acid. In some embodiments, the present invention provides a method of making an esylate salt of compound A. In certain embodiments, the esylate salt of compound A is compound A esylate salt. In certain embodiments, the esylate salt of compound A is Form A of compound A esylate salt. In certain embodiments, the esylate salt of compound A is Form B of compound A esylate salt.

In some embodiments, a suitable acid is toluenesulfonic acid. In some embodiments, the present invention provides a method of making a tosylate salt of compound A. In certain embodiments, the tosylate salt of compound A is compound A tosylate salt. In certain embodiments, the tosylate salt of compound A is Form A of compound A tosylate salt. In certain embodiments, the tosylate salt of compound A is Form B of compound A tosylate salt.

In some embodiments, a suitable acid is glutaric acid. In some embodiments, the present invention provides a method of making a glutarate salt of compound A. In certain embodiments, the glutarate salt of compound A is compound A glutarate salt. In certain embodiments, the glutarate salt of compound A is Form A of compound A glutarate salt.

In some embodiments, a suitable acid is acetic acid. In some embodiments, the present invention provides a method of making an acetate salt of compound A. In certain embodiments, the acetate salt of compound A is compound A acetate salt. In certain embodiments, the acetate salt of compound A is Form A of compound A acetate salt.

In some embodiments, a suitable acid is malonic acid. In some embodiments, the present invention provides a method of making malonate salt of compound A. In certain embodiments, the malonate salt of compound A is compound A malonate salt. In certain embodiments, the malonate salt of compound A is Form A of compound A malonate salt. In certain embodiments, the malonate salt of compound A is Form B of compound A malonate salt.

In some embodiments, a suitable acid is sebacic acid. In some embodiments, the present invention provides a method of making a sebacate salt of compound A. In certain embodiments, the sebacate salt of compound A is compound A sebacate salt. In certain embodiments, the sebacate salt of compound A is Form A of compound A sebacate salt.

In some embodiments, a suitable acid is aceturic acid. In some embodiments, the present invention provides a method of making an aceturate salt of compound A. In certain embodiments, the aceturate salt of compound A is compound A aceturate salt. In certain embodiments, the aceturate salt of compound A is Form A of compound A aceturate salt. In certain embodiments, the aceturate salt of compound A is Form B of compound A aceturate salt.

A suitable solvent may be any solvent system (e.g., one solvent or a mixture of solvents) in which compound A and/or an acid are soluble or are at least partially soluble.

Examples of suitable solvents useful in the present invention include, but are not limited to protic solvents, aprotic solvents, polar aprotic solvent, or mixtures thereof. In certain embodiments, suitable solvents include an ether, an ester, an alcohol, a ketone, or a mixture thereof. In some embodiments, the solvent is one or more organic alcohols. In some embodiments, the solvent is chlorinated. In some embodiments, the solvent is an aromatic solvent.

In certain embodiments, a suitable solvent is methanol, ethanol, isopropanol, or acetone wherein said solvent is anhydrous or in combination with water or heptane. In some embodiments, suitable solvents include tetrahydrofuran, dimethylformamide, dimethylsulfoxide, glyme, diglyme, methyl t-butyl ether, t-butanol, n-butanol, and acetonitrile. In some embodiments, a suitable solvent is ethanol. In some embodiments, a suitable solvent is anhydrous ethanol. In some embodiments, the suitable solvent is MTBE.

In some embodiments, a suitable solvent is ethyl acetate. In some embodiments, a suitable solvent is a mixture of methanol and methylene chloride. In some embodiments, a suitable solvent is a mixture of acetonitrile and water. In certain embodiments, a suitable solvent is methyl acetate, isopropyl acetate, acetone, or tetrahydrofuran. In certain embodiments, a suitable solvent is diethylether. In certain embodiments, a suitable solvent is water. In certain embodiments, a suitable solvent is methyl ethyl ketone. In certain embodiments, a suitable solvent is toluene.

In some embodiments, the present invention provides a method for preparing a salt compound of the general formula X, comprising one or more steps of removing a solvent and adding a solvent. In some embodiments, an added solvent is the same as the solvent removed. In some embodiments, an added solvent is different from the solvent removed. Means of solvent removal are known in the synthetic and chemical arts and include, but are not limited to, any of those described herein and in the Exemplification.

In some embodiments, a method for preparing a salt compound of the general formula X comprises one or more steps of heating or cooling a preparation.

In some embodiments, a method for preparing a salt compound of the general formula X comprises one or more steps of agitating or stirring a preparation.

In some embodiments, a method for preparing a salt compound of the general formula X comprises a step of adding a suitable acid to a solution or slurry of compound A.

In some embodiments, a method for preparing a salt compound of the general formula X comprises a step of heating.

In certain embodiments, a salt compound of formula X precipitates from the mixture. In another embodiment, a salt compound of formula X crystallizes from the mixture. In other embodiments, a salt compound of formula X crystallizes from solution following seeding of the solution (i.e., adding crystals of a salt compound of formula X to the solution).

A salt compound of formula X can precipitate out of the reaction mixture or be generated by removal of part or all of the solvent through methods such as evaporation, distillation, filtration (ex. nanofiltration, ultrafiltration), reverse osmosis, absorption and reaction, by adding an anti-solvent such as heptane, by cooling or by different combinations of these methods.

As described generally above, a salt compound of formula X is optionally isolated. It will be appreciated that a salt compound of formula X may be isolated by any suitable physical means known to one of ordinary skill in the art. In certain embodiments, precipitated solid salt compound of formula X is separated from the supernatant by filtration. In other embodiments, precipitated solid salt compound of formula X is separated from the supernatant by decanting the supernatant.

In certain embodiments, a salt compound of formula X is separated from the supernatant by filtration.

In certain embodiments, an isolated salt compound of formula X is dried in air. In other embodiments, isolated salt compound of formula X is dried under reduced pressure, optionally at elevated temperature.

### Pharmaceutical Compositions and Methods of Administration

In some embodiments, the disclosure is directed to pharmaceutical compositions comprising compound A succinate salt.

The subject pharmaceutical compositions are typically formulated to provide a therapeutically effective amount of a compound of the present disclosure as the active ingredient, or a pharmaceutically acceptable salt, ester, prodrug, solvate, hydrate or derivative thereof. Where desired, the pharmaceutical compositions contain pharmaceutically acceptable salt and/or coordination complex thereof, and one or more pharmaceutically acceptable excipients, carriers, including inert solid diluents and fillers, diluents, including sterile aqueous solution and various organic solvents, permeation enhancers, solubilizers and adjuvants.

The subject pharmaceutical compositions can be administered alone or in combination with one or more other agents, which are also typically administered in the form of pharmaceutical compositions. Where desired, the one or more compounds of the invention and other agent(s) may be mixed into a preparation or both components may be formulated into separate preparations to use them in combination separately or at the same time.

In some embodiments, the concentration of one or more compounds provided in the pharmaceutical compositions of the present invention is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% (or a number in the range defined by and including any two numbers above) w/w, w/v or v/v.

In some embodiments, the concentration of one or more compounds of the invention is greater than 90%, 80%, 70%, 60%, 50%, 40%, 30%, 20%, 19.75%, 19.50%, 19.25%, 19%, 18.75%, 18.50%, 18.25% 18%, 17.75%, 17.50%, 17.25% 17%, 16.75%, 16.50%, 16.25%, 16%, 15.75%, 15.50%, 15.25% 15%, 14.75%, 14.50%, 14.25% 14%, 13.75%, 13.50%, 13.25%, 13%, 12.75%, 12.50%, 12.25%, 12%, 11.75%, 11.50%, 11.25% 11%, 10.75%, 10.50%, 10.25% 10%, 9.75%, 9.50%, 9.25%, 9%, 8.75%, 8.50%, 8.25% 8%, 7.75%, 7.50%, 7.25%, 7%, 6.75%, 6.50%, 6.25%, 6%, 5.75%, 5.50%, 5.25%, 5%, 4.75%, 4.50%, 4.25%, 4%, 3.75%, 3.50%, 3.25%, 3%, 2.75%, 2.50%, 2.25%, 2%, 1.75%, 1.50%, 1.25%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% (or a number in the range defined by and including any two numbers above) w/w, w/v, or v/v.

In some embodiments, the concentration of one or more compounds of the invention is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v.

In some embodiments, the concentration of one or more compounds of the invention is in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v or v/v.

In some embodiments, the amount of one or more compounds of the invention is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g (or a number in the range defined by and including any two numbers above).

In some embodiments, the amount of one or more compounds of the invention is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g (or a number in the range defined by and including any two numbers above).

In some embodiments, the amount of one or more compounds of the invention is in the range of 0.0001-10 g, 0.0005-9 g, 0.001-8 g, 0.005-7 g, 0.01-6 g, 0.05-5 g, 0.1-4 g, 0.5-4 g, or 1-3 g.

The compounds according to the invention are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. An exemplary dosage is 10 to 30 mg per day. The exact dosage will depend upon the route of administration, the form in which the compound is administered, the subject to be treated, the body weight of the subject to be treated, and the preference and experience of the attending physician.

A pharmaceutical composition of the invention typically contains an active ingredient (i.e., a compound of the disclosure) of the present invention or a pharmaceutically acceptable salt and/or coordination complex thereof, and one or more pharmaceutically acceptable excipients, carriers, including but not limited to inert solid diluents and fillers, diluents, sterile aqueous solution and various organic solvents, permeation enhancers, solubilizers and adjuvants.

Described below are non-limiting exemplary pharmaceutical compositions and methods for preparing the same.

### Pharmaceutical Compositions for Oral Administration.

In some embodiments, the invention provides a pharmaceutical composition for oral administration containing a compound of the invention, and a pharmaceutical excipient suitable for oral administration.

In some embodiments, the invention provides a solid pharmaceutical composition for oral administration containing: (i) an effective amount of a compound of the invention; optionally (ii) an effective amount of a second agent; and (iii) a pharmaceutical excipient suitable for oral administration. In some embodiments, the composition further contains: (iv) an effective amount of a third agent.

In some embodiments, the pharmaceutical composition may be a liquid pharmaceutical composition suitable for oral consumption. Pharmaceutical compositions of the invention suitable for oral administration can be presented as discrete dosage forms, such as capsules, cachets, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion. Such dosage forms can be prepared by any of the methods of pharmacy, but all methods include the step of bringing the active ingredient into association with the carrier, which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

This invention further encompasses anhydrous pharmaceutical compositions and dosage forms comprising an active ingredient, since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the invention can be prepared using anhydrous or low moisture containing ingredients and low moisture or low humidity conditions. Pharmaceutical compositions and dosage forms of the invention which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

An active ingredient can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the invention to provide tablets that disintegrate when exposed to an aqueous environment. Too much of a disintegrant may produce tablets which may disintegrate in the bottle. Too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the pharmaceutical composition.

When aqueous suspensions and/or elixirs are desired for oral administration, the active ingredient therein may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactant which can be used to form pharmaceutical compositions and dosage forms of the invention include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions.

Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyl lactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but are not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-lOoleate, Tween 40, Tween 60, sucrose monostearate, sucrose mono laurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In one embodiment, the composition may include a solubilizer to ensure good solubilization and/or dissolution of the compound of the present invention and to minimize precipitation of the compound of the present invention. This can be especially important for compositions for non-oral use, e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, ε-caprolactone and isomers thereof, δ-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a subject using conventional techniques, such as distillation or evaporation. Thus, if present, the solubilizer can be in a weight ratio of 10%, 25%, 50%, 100%, or up to about 200% by weight, based on the combined weight of the drug, and other excipients. If desired, very small amounts of solubilizer may also be used, such as 5%, 2%, 1%, or even less. Typically, the solubilizer may be present in an amount of about 1% to about 100%, more typically about 5% to about 25% by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like.

Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, p-bromophenylsulfonic acid, propionic acid, p-toluenesulfonic acid, salicylic acid, stearic acid, succinic acid, tannic acid, tartaric acid, thioglycolic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, p-bromophenylsulfonic acid, propionic acid, p-toluenesulfonic acid, salicylic acid, stearic acid, succinic acid, tannic acid, tartaric acid, thioglycolic acid, uric acid and the like.

### Pharmaceutical Compositions for Injection.

In some embodiments, the invention provides a pharmaceutical composition for injection containing a compound of the present invention and a pharmaceutical excipient suitable for injection. Components and amounts of agents in the compositions are as described herein.

The forms in which the novel compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the compound of the present invention in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

Pharmaceutical Compositions for Topical (e.g. Transdermal) Delivery.

In some embodiments, the invention provides a pharmaceutical composition for transdermal delivery containing a compound of the present invention and a pharmaceutical excipient suitable for transdermal delivery.

Compositions of the present invention can be formulated into preparations in solid, semisolid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation.

Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another exemplary formulation for use in the methods of the present invention employs transdermal delivery devices (“patches”). Such transdermal patches may be used to provide continuous or discontinuous infusion of a compound of the present invention in controlled amounts, either with or without another agent.

The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents

### Pharmaceutical Compositions for Inhalation.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner.

### Other Pharmaceutical Compositions.

Pharmaceutical compositions may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 20037ybg; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999); all of which are incorporated by reference herein in their entirety.

Administration of the compounds or pharmaceutical composition of the present invention can be affected by any method that enables delivery of the compounds to the site of action. These methods include oral routes, intraduodenal routes, parenteral injection (including intravenous, intraarterial, subcutaneous, intramuscular, intravascular, intraperitoneal or infusion), topical (e.g., transdermal application), rectal administration, via local delivery by catheter or stent or through inhalation. Compounds can also be administered intraadiposally or intrathecally.

The amount of the compound administered will be dependent on the subject being treated, the severity of the disorder or condition, the rate of administration, the disposition of the compound and the discretion of the prescribing physician. However, an effective dosage is in the range of about 0.001 to about 100 mg per kg body weight per day, preferably about 1 to about 35 mg/kg/day, in single or divided doses. For a 70 kg human, this would amount to about 0.05 to 7 g/day, preferably about 0.05 to about 2.5 g/day. In some instances, dosage levels below the lower limit of the aforesaid range may be more than adequate, while in other cases still larger doses may be employed without causing any harmful side effect, e.g. by dividing such larger doses into several small doses for administration throughout the day.

In some embodiments, a compound of the invention is administered in a single dose.

Typically, such administration will be by injection, e.g., intravenous injection, in order to introduce the agent quickly. However, other routes may be used as appropriate. A single dose of a compound of the invention may also be used for treatment of an acute condition.

In some embodiments, a compound of the invention is administered in multiple doses. Dosing may be about once, twice, three times, four times, five times, six times, or more than six times per day. Dosing may be about once a month, once every two weeks, once a week, or once every other day. In another embodiment a compound of the invention and another agent are administered together about once per day to about 6 times per day. In another embodiment the administration of a compound of the invention and an agent continues for less than about 7 days. In yet another embodiment the administration continues for more than about 6, 10, 14, 28 days, two months, six months, or one year. In some cases, continuous dosing is achieved and maintained as long as necessary.

Administration of the compounds of the invention may continue as long as necessary. In some embodiments, a compound of the invention is administered for more than 1, 2, 3, 4, 5, 6, 7, 14, or 28 days. In some embodiments, a compound of the invention is administered for less than 28, 14, 7, 6, 5, 4, 3, 2, or 1 day. In some embodiments, a compound of the invention is administered chronically on an ongoing basis, e.g., for the treatment of chronic effects.

An effective amount of a compound of the invention may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, or as an inhalant.

The compositions of the invention may also be delivered via an impregnated or coated device such as a stent, for example, or an artery-inserted cylindrical polymer. Such a method of administration may, for example, aid in the prevention or amelioration of restenosis following procedures such as balloon angioplasty. Without being bound by theory, compounds of the invention may slow or inhibit the migration and proliferation of smooth muscle cells in the arterial wall which contribute to restenosis. A compound of the invention may be administered, for example, by local delivery from the struts of a stent, from a stent graft, from grafts, or from the cover or sheath of a stent. In some embodiments, a compound of the invention is admixed with a matrix. Such a matrix may be a polymeric matrix, and may serve to bond the compound to the stent. Polymeric matrices suitable for such use, include, for example, lactone-based polyesters or copolyesters such as polylactide, polycaprolactonglycolide, polyorthoesters, polyanhydrides, polyaminoacids, polysaccharides, polyphosphazenes, poly (ether-ester) copolymers (e.g. PEO-PLLA); polydimethylsiloxane, poly (ethylene-vinylacetate), acrylate-based polymers or copolymers (e.g. polyhydroxyethyl methylmethacrylate, polyvinyl pyrrolidinone), fluorinated polymers such as polytetrafluoroethylene and cellulose esters. Suitable matrices may be nondegrading or may degrade with time, releasing the compound or compounds. Compounds of the invention may be applied to the surface of the stent by various methods such as dip/spin coating, spray coating, dip-coating, and/or brush-coating. The compounds may be applied in a solvent and the solvent may be allowed to evaporate, thus forming a layer of compound onto the stent. Alternatively, the compound may be located in the body of the stent or graft, for example in microchannels or micropores. When implanted, the compound diffuses out of the body of the stent to contact the arterial wall. Such stents may be prepared by dipping a stent manufactured to contain such micropores or microchannels into a solution of the compound of the invention in a suitable solvent, followed by evaporation of the solvent. Excess drug on the surface of the stent may be removed via an additional brief solvent wash. In yet other embodiments, compounds of the invention may be covalently linked to a stent or graft. A covalent linker may be used which degrades in vivo, leading to the release of the compound of the invention. Any bio-labile linkage may be used for such a purpose, such as ester, amide or anhydride linkages. Compounds of the invention may additionally be administered intravascularly from a balloon used during angioplasty. Extravascular administration of the compounds via the pericard or via advential application of formulations of the invention may also be performed to decrease restenosis.

A variety of stent devices which may be used as described are disclosed, for example, in the following references, all of which are hereby incorporated by reference: U.S. Pat. Nos. 5,451,233; 5,040,548; 5,061,273; 5,496,346; 5,292,331; 5,674,278; 3,657,744; 4,739,762; 5,195,984; 5,292,331; 5,674,278; 5,879,382; 6,344,053.

The compounds of the invention may be administered in dosages. It is known in the art that due to intersubject variability in compound pharmacokinetics, individualization of dosing regimen is necessary for optimal therapy. Dosing for a compound of the invention may be found by routine experimentation in light of the instant disclosure.

When a compound of the invention is administered in a composition that comprises one or more agents, and the agent has a shorter half-life than the compound of the invention unit dose forms of the agent and the compound of the invention may be adjusted accordingly.

The subject pharmaceutical composition may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc.

Exemplary parenteral administration forms include solutions or suspensions of active compound in sterile aqueous solutions, for example, aqueous propylene glycol or dextrose solutions. Such dosage forms can be suitably buffered, if desired.

### Methods of Use

The method typically comprises administering to a subject a therapeutically effective amount of a compound of the invention. The therapeutically effective amount of the subject combination of compounds may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of proliferation or downregulation of activity of a target protein. The specific dose will vary depending on the particular compounds chosen, the dosing regimen to be followed, whether it is administered in combination with other compounds, timing of administration, the tissue to which it is administered, and the physical delivery system in which it is carried.

As used herein, the term “IC₅₀” refers to the half maximal inhibitory concentration of an inhibitor in inhibiting biological or biochemical function. This quantitative measure indicates how much of a particular inhibitor is needed to inhibit a given biological process (or component of a process, i.e., an enzyme, cell, cell receptor or microorganism) by half. In other words, it is the half maximal (50%) inhibitory concentration (IC) of a substance (50% IC, or IC₅₀). EC₅₀ refers to the plasma concentration required for obtaining 50%> of a maximum effect in vivo.

In some embodiments, the subject methods utilize a CDK inhibitor with an IC50 value of about or less than a predetermined value, as ascertained in an in vitro assay. In some embodiments, the CDK inhibitor inhibits CDK a with an IC₅₀ value of about 1 nM or less, 2 nM or less, 5 nM or less, 7 nM or less, 10 nM or less, 20 nM or less, 30 nM or less, 40 nM or less, 50 nM or less, 60 nM or less, 70 nM or less, 80 nM or less, 90 nM or less, 100 nM or less, 120 nM or less, 140 nM or less, 150 nM or less, 160 nM or less, 170 nM or less, 180 nM or less, 190 nM or less, 200 nM or less, 225 nM or less, 250 nM or less, 275 nM or less, 300 nM or less, 325 nM or less, 350 nM or less, 375 nM or less, 400 nM or less, 425 nM or less, 450 nM or less, 475 nM or less, 500 nM or less, 550 nM or less, 600 nM or less, 650 nM or less, 700 nM or less, 750 nM or less, 800 nM or less, 850 nM or less, 900 nM or less, 950 nM or less, 1 μM or less, 1.1 μM or less, 1.2 μM or less, 1.3 μM or less, 1.4 μM or less, 1.5 μM or less, 1.6 μM or less, 1.7 μM or less, 1.8 μM or less, 1.9 μM or less, 2 μM or less, 5 μM or less, 10 μM or less, 15 μM or less, 20 μM or less, 25 μM or less, 30 μM or less, 40 μM or less, 50 μM, 60 μM, 70 μM, 80 μM, 90 μM, 100 μM, 200 μM, 300 μM, 400 μM, or 500 μM, or less, (or a number in the range defined by and including any two numbers above).

In some embodiments, the CDK inhibitor selectively inhibits CDK a with an IC₅₀ value that is at least 2, 3, 4, 5, 6, 7, 8, 9, 10, 15, 20, 25, 30, 35, 40, 45, 50, 100, or 1000 times less (or a number in the range defined by and including any two numbers above) than its IC₅₀ value against one, two, or three other CDKs.

In some embodiments, the CDK inhibitor selectively inhibits CDK a with an IC₅₀ value that is less than about 1 nM, 2 nM, 5 nM, 7 nM, 10 nM, 20 nM, 30 nM, 40 nM, 50 nM, 60 nM, 70 nM, 80 nM, 90 nM, 100 nM, 120 nM, 140 nM, 150 nM, 160 nM, 170 nM, 180 nM, 190 nM, 200 nM, 225 nM, 250 nM, 275 nM, 300 nM, 325 nM, 350 nM, 375 nM, 400 nM, 425 nM, 450 nM, 475 nM, 500 nM, 550 nM, 600 nM, 650 nM, 700 nM, 750 nM, 800 nM, 850 nM, 900 nM, 950 nM, 1 μM, 1.1 μM, 1.2 μM, 1.3 μM, 1.4 μM, 1.5 μM, 1.6 μM, 1.7 μM, 1.8 μM, 1.9 μM, 2 μM, 5 μM, 10 μM, 15 μM, 20 μM, 25 μM, 30 μM, 40 μM, 50 μM, 60 μM, 70 μM, 80 μM, 90 μM, 100 μM, 200 μM, 300 μM, 400 μM, or 500 μM (or in the range defined by and including any two numbers above), and said IC50 value is at least 2, 3, 4, 5, 6, 7, 8, 9, 10, 15, 20, 25, 30, 35, 40, 45, 50, 100, or 1000 times less (or a number in the range defined by and including any two numbers above) than its IC₅₀ value against one, two or three other CDKs.

The subject methods are useful for treating a disease condition associated with CDK. Any disease condition that results directly or indirectly from an abnormal activity or expression level of CDK can be an intended disease condition.

Different disease conditions associated with CDK have been reported. CDK has been implicated, for example, auto-immune diseases, neurodegeneration (such as Parkinson's disease, Alzheimer's disease and ischaemia), inflammatory diseases, viral infections and cancer such as, for example, colon cancer, breast cancer, small-cell lung cancer, non-small-cell lung cancer, bladder cancer, ovarian cancer, prostate cancer, chronic lymphoid leukemia, lymphoma, myeloma, acute myeloid leukemia, or pancreatic cancer.

Non-limiting examples of such conditions include but are not limited to Acanthoma, Acinic cell carcinoma, Acoustic neuroma, Acral lentiginous melanoma, Acrospiroma, Acute eosinophilic leukemia, Acute lymphoblastic leukemia, Acute lymphocytic leukemia, Acute megakaryoblastic leukemia, Acute monocytic leukemia, Acute myeloblasts leukemia with maturation, Acute myeloid dendritic cell leukemia, Acute myeloid leukemia, Acute myelogenous leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epidermoid cancer, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Head and Neck Cancer, Head and neck cancer, Heart cancer, Hemoglobinopathies such as b-thalassemia and sickle cell disease (SCD), Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mastocytosis, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplasia Disease, Myelodysplasia Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene onChromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor, or any combination thereof.

In some embodiments, said method is for treating a disease selected from the group consisting of tumor angiogenesis, chronic inflammatory disease such as rheumatoid arthritis, atherosclerosis, inflammatory bowel disease, skin diseases such as psoriasis, eczema, and scleroderma, diabetes, diabetic retinopathy, retinopathy of prematurity, age-related macular degeneration, hemangioma, glioma, melanoma, Kaposi's sarcoma and ovarian, breast, lung, pancreatic, prostate, colon and epidermoid cancer.

In other embodiments, said method is for treating a disease selected from breast cancer, lung cancer, pancreatic cancer, prostate cancer, colon cancer, ovarian cancer, uterine cancer, or cervical cancer.

In other embodiments, said method is for treating a disease selected from leukemia such as acute myeloid leukemia (AML), acute lymphocytic leukemia, chronic lymphocytic leukemia, chronic myeloid leukemia, hairy cell leukemia, myelodysplasia, myeloproliferative disorders, acute myelogenous leukemia (AML), chronic myelogenous leukemia (CML), mastocytosis, chronic lymphocytic leukemia (CLL), multiple myeloma (MM), myelodysplastic syndrome (MDS) or epidermoid cancer.

Compounds of the disclosure, as well as pharmaceutical compositions comprising them, can be administered to treat any of the described diseases, alone or in combination with a medical therapy. Medical therapies include, for example, surgery and radiotherapy (e.g., gamma-radiation, neutron beam radiotherapy, electron beam radiotherapy, proton therapy, brachytherapy, systemic radioactive isotopes).

In other aspects, compounds of the disclosure, as well as pharmaceutical compositions comprising them, can be administered to treat any of the described diseases, alone or in combination with one or more other agents.

In other methods, the compounds of the disclosure, as well as pharmaceutical compositions comprising them, can be administered in combination with agonists of nuclear receptors agents.

In other methods, the compounds of the disclosure, as well as pharmaceutical compositions comprising them, can be administered in combination with antagonists of nuclear receptors agents.

In other methods, the compounds of the disclosure, as well as pharmaceutical compositions comprising them, can be administered in combination with an anti-proliferative agent.

In some embodiments, the disclosure is directed to methods for treating a CDK4-mediated and a CDK6-mediated disorder in a patient in need thereof, comprising administering to said patient a compound of Formula I, including all subgenera described herein, or a pharmaceutically acceptable salt thereof, or a pharmaceutical composition comprising the compound of Formula I, including all subgenera described herein.

In some embodiments, the CDK4-mediated and CDK6-mediated disorder is a cancer. In some embodiments, the cancer is breast cancer, malignant brain tumors, colon cancer, small-cell lung cancer, non-small-cell lung cancer, bladder cancer, ovarian cancer, prostate cancer, chronic lymphoid leukemia, lymphoma, myeloma, acute myeloid leukemia, secondary pancreatic cancer or secondary brain metastases.

In some embodiments, the cancer is breast cancer. In some embodiments, the cancer is malignant brain tumors. In some embodiments, the cancer is colon cancer. In some embodiments, the cancer is small-cell lung cancer. In some embodiments, the cancer is non-small-cell lung cancer. In some embodiments, the cancer is bladder cancer. In some embodiments, the cancer is ovarian cancer.

In some embodiments, the cancer is prostate cancer. In some embodiments, the cancer is chronic lymphoid leukemia. In some embodiments, the cancer is lymphoma. In some embodiments, the cancer is myeloma. In some embodiments, the cancer is acute myeloid leukemia. In some embodiments, the cancer is secondary pancreatic cancer. In some embodiments, the cancer is secondary brain metastases.

In some embodiments, the breast cancer is HR+/HER2− or HR+/HER2+ advanced or metastatic breast cancer. In some embodiments, the breast cancer is HR+/HER2− advanced breast cancer. In some embodiments, the breast cancer is HR+/HER2− metastatic breast cancer. In some embodiments, the breast cancer is HR+/HER2+ advanced breast cancer. In some embodiments, the breast cancer is HR+/HER2+ metastatic breast cancer.

In some embodiments, the malignant brain tumors are glioblastoma, astrocytoma, or pontine glioma. In some embodiments, the malignant brain tumors are a glioblastoma. In some embodiments, the malignant brain tumors are an astrocytoma. In some embodiments, the malignant brain tumors are a pontine glioma.

In some embodiments, the patient is administered a pharmaceutical composition comprising a compound of Formula I, including all subgenera described herein, or a pharmaceutically acceptable salt thereof. In some embodiments, the administration is oral administration.

### Combination Therapies

For treating cancer and other proliferative diseases, the compounds of the invention can be used in combination with chemotherapeutic agents, agonists or antagonists of nuclear receptors, or other anti-proliferative agents. The compounds of the invention can also be used in combination with a medical therapy such as surgery or radiotherapy, e.g., gamma-radiation, neutron beam radiotherapy, electron beam radiotherapy, proton therapy, brachytherapy, and systemic radioactive isotopes. Examples of suitable chemotherapeutic agents include any of: abarelix, aldesleukin, alemtuzumab, alitretinoin, allopurinol, all-trans retinoic acid, altretamine, anastrozole, arsenic trioxide, asparaginase, azacitidine, bendamustine, bevacizumab, bexarotene, bleomycin, bortezombi, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, epirubicin, erlotinib, estramustine, etoposide phosphate, etoposide, exemestane, fentanyl citrate, filgrastim, floxuridine, fludarabine, fluorouracil, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, oxaliplatin, paclitaxel, pamidronate, panobinostat, panitumumab, pegaspargase, pegfilgrastim, pemetrexed disodium, pentostatin, pipobroman, plicamycin, procarbazine, quinacrine, rasburicase, rituximab, ruxolitinib, sorafenib, streptozocin, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, vorinstat and zoledronate.

In some embodiments, the compounds of the invention can be used in combination with a therapeutic agent that targets an epigenetic regulator. Examples of epigenetic regulators include bromodomain inhibitors, the histone lysine methyltransferase inhibitors, histone arginine methyl transferase inhibitors, histone demethylase inhibitors, histone deacetylase inhibitors, histone acetylase inhibitors, and DNA methyltransferase inhibitors. Histone deacetylase inhibitors include, e.g., vorinostat. Histone arginine methyl transferase inhibitors include inhibitors of protein arginine methyltransferases (PRMTs) such as PRMT5, PRMT1 and PRMT4. DNA methyltransferase inhibitors include inhibitors of DNMT1 and DNMT3.

For treating cancer and other proliferative diseases, the compounds of the invention can be used in combination with targeted therapies, including JAK kinase inhibitors (e.g. Ruxolitinib), PI3 kinase inhibitors including PI3K-delta selective and broad spectrum PI3K inhibitors, MEK inhibitors, Cyclin Dependent kinase inhibitors, including CDK4/6 inhibitors and CDK9 inhibitors, BRAF inhibitors, mTOR inhibitors, proteasome inhibitors (e.g. Bortezomib, Carfilzomib), HDAC inhibitors (e.g. panobinostat, vorinostat), DNA methyl transferase inhibitors, dexamethasone, bromo and extra terminal family member (BET) inhibitors, BTK inhibitors (e.g. ibrutinib, acalabrutinib), BCL2 inhibitors (e.g. venetoclax), dual BCL2 family inhibitors (e.g. BCL2/BCLxL), PARP inhibitors, FLT3 inhibitors, or LSD1 inhibitors.

In some embodiments, the inhibitor of an immune checkpoint molecule is an inhibitor of PD-1, e.g., an anti-PD-1 monoclonal antibody. In some embodiments, the anti-PD-1 monoclonal antibody is nivolumab, pembrolizumab (also known as MK-3475), or PDR001. In some embodiments, the anti-PD-1 monoclonal antibody is nivolumab or pembrolizumab. In some embodiments, the anti-PD1 antibody is pembrolizumab. In some embodiments, the inhibitor of an immune checkpoint molecule is an inhibitor of PD-L1, e.g., an anti-PD-L1 monoclonal antibody. In some embodiments, the anti-PD-L1 monoclonal antibody is atezolizumab, durvalumab, or BMS-935559. In some embodiments, the inhibitor of an immune checkpoint molecule is an inhibitor of CTLA-4, e.g., an anti-CTLA-4 antibody. In some embodiments, the anti-CTLA-4 antibody is ipilimumab.

In some embodiments, the agent is an alkylating agent, a proteasome inhibitor, a corticosteroid, or an immunomodulatory agent. Examples of an alkylating agent include cyclophosphamide (CY), melphalan (MEL), and bendamustine. In some embodiments, the proteasome inhibitor is carfilzomib. In some embodiments, the corticosteroid is dexamethasone (DEX). In some embodiments, the immunomodulatory agent is lenalidomide (LEN) or pomalidomide (POM).

For treating autoimmune or inflammatory conditions, the compound of the invention can be administered in combination with a corticosteroid such as triamcinolone, dexamethasone, fluocinolone, cortisone, prednisolone, or flumetholone.

For treating autoimmune or inflammatory conditions, the compound of the invention can be administered in combination with an immune suppressant such as fluocinolone acetonide (Retisert®), rimexolone (AL-2178, Vexol, Alcon), or cyclosporine (Restasis®).

In some embodiments, the disclosure is directed to methods described herein, further comprising administering an additional therapeutic agent to the patient. In some embodiments, the additional therapeutic agent is a PRMT5 inhibitor, a HER2 kinase inhibitor, an aromatase inhibitor, an estrogen receptor antagonist or an alkylating agent.

In some embodiments, the additional therapeutic agent is a PRMT5 inhibitor. In some embodiments, the additional therapeutic agent is a HER2 kinase inhibitor. In other embodiments, the additional therapeutic agent is an aromatase inhibitor. In other embodiments, the additional therapeutic agent is an estrogen receptor antagonist. In yet other embodiments, the additional therapeutic agent is an alkylating agent.

In some embodiments, the aromatase inhibitor is letrozole. In some embodiments, the estrogen receptor antagonist is fulvestrant. In other embodiments, the alkylating agent is temozolomide.

In yet other embodiments, the PRMT5 inhibitor is a compound disclosed in US Published Patent Application No. 2020/0148692 (filed Jan. 16, 2020); US Published Patent Application No. 2019/0284193 (filed Apr. 5, 2019); and US Published Patent Application No. 2019/0048014 (filed Aug. 9, 2018); each of which is hereby incorporated herein in its entirety.

In some embodiments, the PRMT5 inhibitor is:
(2R,3R,4S,5S)-2-(4-amino-7H-pyrrolo[2,3-d]pyrimidin-7-yl)-5-((R)-6-chloroiso-chroman-1-yl)tetrahydrofuran-3,4-diol, or a pharmaceutically acceptable salt or solvate thereof;
(2R,3R,4S,5S)-2-(4-amino-7H-pyrrolo[2,3-d]pyrimidin-7-yl)-5-((R)-7-chloroiso-chroman-1-yl)tetrahydrofuran-3,4-diol, or a pharmaceutically acceptable salt or solvate thereof;
(2R,3R,4S,5S)-2-(4-amino-7H-pyrrolo[2,3-d]pyrimidin-7-yl)-5-((R)-5-chloroiso-chroman-1-yl)tetrahydrofuran-3,4-diol, or a pharmaceutically acceptable salt or solvate thereof;
(2R,3R,4S,5S)-2-(4-amino-7H-pyrrolo[2,3-d]pyrimidin-7-yl)-5-((R)-6,7-difluoroiso-chroman-1-yl)tetrahydrofuran-3,4-diol, or a pharmaceutically acceptable salt or solvate thereof;
(2R,3R,4S,5S)-2-(4-amino-7H-pyrrolo[2,3-d]pyrimidin-7-yl)-5-((R)-5,6-difluoroiso-chroman-1-yl)tetrahydrofuran-3,4-diol, or a pharmaceutically acceptable salt or solvate thereof;
(2R,3R,4S,5S)-2-(4-amino-7H-pyrrolo[2,3-d]pyrimidin-7-yl)-5-((R)-6-chloro-5-fluoroisochroman-1-yl)tetrahydrofuran-3,4-diol, or a pharmaceutically acceptable salt or solvate thereof;
(2R,3R,4S,5S)-2-(4-amino-5-fluoro-7H-pyrrolo[2,3-d]pyrimidin-7-yl)-5-((R)-6-chloroisochroman-1-yl)tetrahydrofuran-3,4-diol, or a pharmaceutically acceptable salt or solvate thereof;
(2R,3R,4S,5S)-2-(4-amino-7H-pyrrolo[2,3-d]pyrimidin-7-yl)-5-((R)-6,7-dichloroiso-chroman-1-yl)tetrahydrofuran-3,4-diol, or a pharmaceutically acceptable salt or solvate thereof;
(2S,3S,4R,5R)-2-((R)-6-chloroisochroman-1-yl)-5-(4-methyl-7H-pyrrolo[2,3-d]pyrimidin-7-yl)tetrahydrofuran-3,4-diol, or a pharmaceutically acceptable salt or solvate thereof;
(2S,3S,4R,5R)-2-((R)-6,7-difluoroisochroman-1-yl)-5-(4-methyl-7H-pyrrolo[2,3-d]pyrimidin-7-yl)tetrahydrofuran-3,4-diol, or a pharmaceutically acceptable salt or solvate thereof;
(2S,3S,4R,5R)-2-((R)-5,6-difluoroisochroman-1-yl)-5-(4-methyl-7H-pyrrolo[2,3-d]pyrimidin-7-yl)tetrahydrofuran-3,4-diol, or a pharmaceutically acceptable salt or solvate thereof;
(2S,3S,4R,5R)-2-((R)-6-chloroisochroman-1-yl)-5-(5-fluoro-4-methyl-7H-pyrrolo[2,3-d]pyrimidin-7-yl)tetrahydrofuran-3,4-diol, or a pharmaceutically acceptable salt or solvate thereof;
(2S,3S,4R,5R)-2-((R)-6,7-dichloroisochroman-1-yl)-5-(4-methyl-7H-pyrrolo[2,3-d]pyrimidin-7-yl)tetrahydrofuran-3,4-diol, or a pharmaceutically acceptable salt or solvate thereof.

In some embodiments, the PRMT5 inhibitor is (2S,3S,4R,5R)-2-((R)-6-chloroiso-chroman-1-yl)-5-(4-methyl-7H-pyrrolo[2,3-d]pyrimidin-7-yl)tetrahydrofuran-3,4-diol, or a pharmaceutically acceptable salt or solvate thereof.

All features of each of the aspects of the invention apply to all other aspects mutatis mutandis.

In order that the invention described herein may be more fully understood, the following examples are set forth. It should be understood that these examples are for illustrative purposes only and are not to be construed as limiting this invention in any manner.

### EXEMPLIFICATION

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

### Example A—General Preparation of Compound A

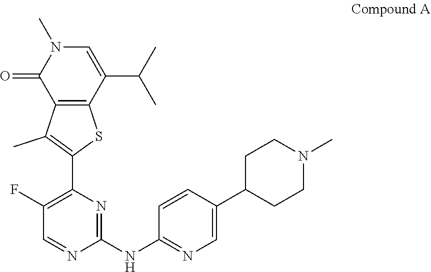 [SMILES: Cc1c(-c2nc(Nc3ccc(C4CCN(C)CC4)cn3)ncc2F)sc2c(C(C)C)cn(C)c(=O)c12]

The title compound was prepared according to the steps and intermediates described below and in the '323 application, the entirety of which is incorporated herein by reference.

### Step 1. tert-Butyl 6-nitro-3′,6′-dihydro-[3,4′-bipyridine]-1′(2′H)-carboxylate

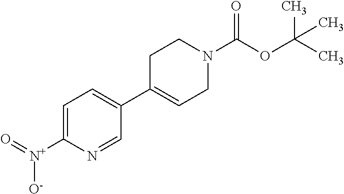 [SMILES: CC(C)(C)OC(=O)N1CC=C(c2ccc([N+](=O)[O-])nc2)CC1]

N-Boc-1,2,3,6-tetrahydropyridine-4-boronic acid pinacol ester (12.0 g, 38.8 mmol), 5-bromo-2-nitropyridine (7.80 g, 38.4 mmol), sodium carbonate (15.9 g, 115 mmol), and 1,1′-bis(diphenylphosphino)ferrocene]dichloropalladium (II) (2.51 g, 3.84 mmol) were suspended in 1,4-dioxane (120 mL) and water (40.0 mL) under inert atmosphere. The reaction mixture was heated at 100° C. for 6 h. The reaction mixture was cooled to room temperature and diluted with water (100 mL). The mixture was extracted with EtOAc (100 mL×3). The organic layers were combined, washed with brine, dried over sodium sulfate, filtered, and concentrated. The crude residue was purified by silica gel chromatography using EtOAc in heptanes (10-60% with 0.1% Et₃N) to afford the title compound (9.20 g, 30.1 mmol, 78.4% yield) as a brown solid. LCMS calc. for C₁₅H₂₀N₃O₄ [M+H]⁺: m/z=306.1; Found: 306.1.

### Step 2. 6-Nitro-1′,2′,3′,6′-tetrahydro-3,4′-bipyridine

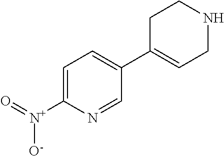 [SMILES: O=[N+]([O-])c1ccc(C2=CCNCC2)cn1]

tert-Butyl 6-nitro-3′,6′-dihydro-[3,4′-bipyridine]-1′ (2′H)-carboxylate (11.0 g, 36.0 mmol) was dissolved in DCM (30.0 mL) and cooled to 0° C. Trifluoroacetic acid (10.0 mL, 131 mmol) was added slowly at 0° C. The reaction mixture was warmed to room temperature and stirred for 5 h. The reaction mixture was concentrated under reduced pressure and then diluted with water (10.0 mL). The solution was lyophilized and used without further purification to afford the crude TFA salt of the title compound (18.6 g) as a yellow solid. LCMS calc. for C₁₀H₁₂N₃O₂ [M+H]: m/z=206.1; Found: 206.0.

### Step 3. 1′-Methyl-6-nitro-1′,2′,3′,6′-tetrahydro-3,4′-bipyridine

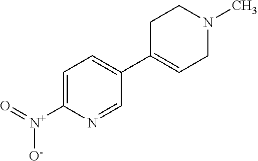 [SMILES: CN1CC=C(c2ccc([N+](=O)[O-])nc2)CC1]

Formaldehyde (24.9 mL, 335 mmol, 37 wt % in H₂O) and the crude TFA salt of 6-nitro-1′,2′,3′,6′-tetrahydro-3,4′-bipyridine (18.6 g) were dissolved in DCM (60 mL) at room temperature. The mixture was stirred for 30 min, and the reaction was cooled to 0° C. Sodium triacetoxyborohydride (14.2 g, 66.9 mmol) was added portion-wise at 0° C. The resulting reaction mixture was allowed to warm to room temperature and stirred overnight. The reaction was quenched with sat. sodium bicarbonate (aq) (60.0 mL), and sodium carbonate was added until gas evolution was no longer observed. The organic phase was separated, and the aqueous layer was extracted with DCM (30.0 mL×2). The organic layers were combined, dried over sodium sulfate, filtered, and concentrated under reduced pressure. The reaction sequence in steps 2-3 was repeated with an additional portion of tert-butyl 6-nitro-3′,6′-dihydro-[3,4′-bipyridine]-1′ (2′H)-carboxylate (2.5 mmol in Step 2), and the crude material from both sequences was combined for purification. Purification by silica gel chromatography using MeOH in DCM (0-50%) afforded the freebase of the title compound (8.10 g, 36.9 mmol, quantitative yield over two steps) as a yellow solid. ¹H NMR (300 MHz, DMSO-d₆) δ 8.74 (d, J=2.1 Hz, 1H), 8.29-8.16 (m, 2H), 6.57 (t, J=3.6 Hz, 1H), 3.09-3.03 (m, 2H), 2.60-2.52 (m, 4H), 2.27 (s, 3H). LCMS calc. for C₁₁H₁₄N₃O₂ [M+H]⁺: m/z=220.1; Found: 220.0.

The freebase was dissolved in EtOAc (50.0 mL), DCM (5.00 mL), and MeOH (5.00 mL). Then a solution of HCl (37.0 mL, 74.0 mmol, 2N in iPrOAc) was added. The reaction mixture was stirred for 2 h at room temperature. The precipitate was collected by filtration and dried under reduced pressure to afford the HCl salt of the title compound (9.46 g, 32.4 mmol, 90.1% yield) as a white solid.

### Step 4. 5-(1-Methylpiperidin-4-yl) pyridin-2-amine

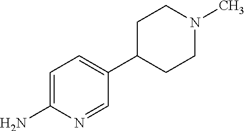

In a 500 mL reaction vessel, l′-methyl-6-nitro-1′,2′,3′,6′-tetrahydro-3,4′-bipyridine, HCl salt (9.46 g, 32.4 mmol) was dissolved in MeOH (150 mL). Palladium on carbon (0.875 g, 0.822 mmol, 10 wt %) and glacial acetic acid (1.00 mL, 17.5 mmol) were added sequentially at room temperature. The reaction vessel was sealed in a Parr shaker, and the vessel was charged with hydrogen (60 psi). The reaction mixture was mixed overnight. The atmosphere of hydrogen was removed, the mixture was filtered, and the filtrate was concentrated under reduced pressure. The dark residue was dissolved in DCM (30.0 mL) and neutralized with sodium hydroxide (3.39 g, 84.8 mmol). The organic layer was washed with water (30.0 mL×2), dried over sodium sulfate, filtered, and concentrated under reduced pressure. The crude residue was purified by silica gel chromatography using MeOH in DCM (0-50% with 0.1% Et₃N) to afford the title compound (6.10 g, 31.9 mmol, 98.5% yield) as an off-white solid. ¹H NMR (300 MHz, DMSO-d₆) δ 7.74 (d, J=2.2 Hz, 1H), 7.23 (dd, J=8.5, 2.4 Hz, 1H), 6.36 (d, J=8.5 Hz, 1H), 5.62 (s, 2H), 2.81 (d, J=11.4 Hz, 2H), 2.40-2.16 (m, 1H), 2.15 (s, 3H), 2.00-1.75 (m, 2H), 1.64-1.47 (m, 4H). LCMS calc. for C₁₁H₁₈N₃ [M+H]: m/z=192.1; Found: 192.1.

### Step 5. 2-(2-Chloro-5-fluoropyrimidin-4-yl)-7-isopropyl-3,5-dimethylthieno[3,2-c]pyridin-4 (5H)-one

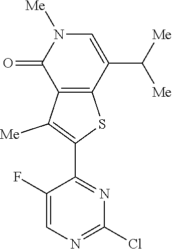 [SMILES: Cc1c(-c2nc(Cl)ncc2F)sc2c(C(C)C)cn(C)c(=O)c12]

n-Butyllithium (5.20 mL, 13.0 mmol, 2.5 M in hexanes) was added dropwise to a solution of 2-bromo-7-isopropyl-3,5-dimethylthieno[3,2-c]pyridin-4 (5H)-one (2.60 g, 8.66 mmol) at −78° C. The reaction mixture was stirred for 20 min at −78° C. 2-Chloro-5-fluoropyrimidine (1.38 g, 10.4 mmol) was then added in a single portion. The reaction mixture was stirred for 30 min at −78° C. The reaction was quenched with sat. NH₄Cl (aq) and diluted with DCM (30.0 mL). The mixture was allowed to warm to room temperature, and the two phases were separated. The organic layer was removed, and the aqueous layer was extracted with DCM (30.0 mL×2). The combined organic layers were washed with brine, dried over sodium sulfate, and concentrated.

To the crude residue in THF (30.0 mL) was added 2,3-dichloro-5,6-dicyano-p-benzoquinone (1.86 g, 8.20 mmol). The reaction mixture was stirred for 30 min at room temperature. The reaction mixture was diluted with DCM (30.0 mL), washed with hot potassium carbonate (10 wt % aq.) (30.0 mL×2), dried over sodium sulfate, and concentrated. The crude residue was purified by prep-HPLC using a C18 column (20-100% MeCN/0.1% TFA (aq.)) to afford the TFA salt of the title compound (1.35 g, 2.90 mmol, 35.3% yield) as an off-white solid. LCMS calc. for C₁₆H₁₆ClFN₃OS [M+H]⁺: m/z=352.1, 354.1; Found: 352.0, 353.9.

### Step 6. 2-(5-Fluoro-2-((5-(1-methylpiperidin-4-yl) pyridin-2-yl)amino)pyrimidin-4-yl)-7-isopropyl-3,5-dimethylthieno[3,2-c]pyridin-4 (5H)-one

To a solution of 2-(2-chloro-5-fluoropyrimidin-4-yl)-7-isopropyl-3,5-dimethyl-thieno[3,2-c]pyridin-4 (5H)-one, TFA salt (1.20 g, 2.58 mmol) and 5-(1-methylpiperidin-4-yl) pyridin-2-amine (0.783 g, 4.09 mmol) in 1,4-dioxane (24.0 mL) was added K₃PO₄ (2.17 g, 10.2 mmol) and XPhos Pd G2 (268 mg, 0.341 mmol, CAS 1310584-14-5). The reaction vessel was sealed, and the mixture was degassed with N₂ three times. The reaction mixture was heated at 100° C. overnight. The reaction mixture was cooled to room temperature, filtered, and concentrated under reduced pressure. The dark residue was purified by prep-HPLC on a C18 column (10-50% MeCN/0.1% TFA (aq.)) to afford the title compound as the TFA salt. The TFA salt was neutralized with sat. NaHCO₃(aq) (20.0 mL), and the aqueous layer was extracted with DCM (20.0 mL×3). The organic layers were combined, washed with brine, dried over sodium sulfate, filtered, and concentrated under reduced pressure. The residue was dissolved in 1N HCl (2.20 mL, 2.20 mmol, 1.05 equiv), and the solvent was removed by lyophilization to afford the HCl salt of the title compound (1.21 g, 2.23 mmol, 86.5% yield) as a yellow solid. ¹H NMR (300 MHz, DMSO-d₆) δ 10.46 (br s, 1H), 10.31 (br s, 1H), 8.75 (d, J=2.5 Hz, 1H), 8.21 (d, J=2.4 Hz, 1H), 8.12 (d, J=8.7 Hz, 1H), 7.71 (dd, J=8.8, 2.4 Hz, 1H), 7.55 (s, 1H), 3.52 (s, 3H), 3.46 (s, 2H), 3.20-2.97 (m, 2H), 2.95-2.84 (m, 2H), 2.77 (d, J=4.3 Hz, 3H), 2.69 (d, J=3.5 Hz, 3H), 2.06-1.93 (m, 4H), 1.31 (d, J=6.8 Hz, 6H). LCMS calc. for C₂₇H₃₂FN₆OS [M+H]⁺: m/z=507.2; Found: 507.2.

### General Procedures for Examples 1-8

X-Ray Powder Diffraction (XRPD) Data. XRPD data was collected with the following parameters:
Instrument: Rigaku Smart lab SE
X-Ray Wavelength: Cu, Kα, Kα1(Å): 1.540598, Kα2(Å): 1.544426
Kα2:Kα1 intensity ratio: 0.50
X-Ray Tube Setting: 40 kV, 15 mA, Scan Mode 1D
Scan Range (2 Theta): 3°-40°, Step Size (2 Theta): 0.02° with Scan Speed (2 Theta) 10°/min.

Thermogravimetric Analysis (TGA). TGA data was collected with the following parameters:
Instrument: TA, TGA500
Method: Ramp, Temperature Range RT-300° C., Heating Rate 10° C./min
Purge Gas: N2 (>99.999%)
Pan Type: Aluminum, Open.

Differential Scanning calorimetry (DSC): DSC data was collected with the following parameters:
Instrument: TA, TGA500
Method: Ramp, Temperature Range RT-300° C., Heating Rate 10° C./min
Purge Gas: N2 (>99.999%)
Pan Type: Aluminum, Open.

Polarized Light Microscopy (PLM): PLM data was collected with the following parameters:
Instrument: Olympus BX53
Method: The sample was dispersed with methyl silicone oil on a glass slide.

### Example 1—Form A of Compound A Free Base

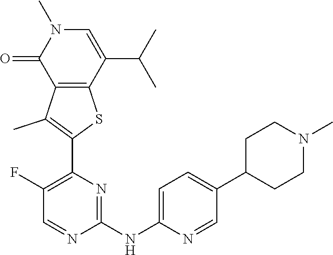 [SMILES: Cc1c(-c2nc(Nc3ccc(C4CCN(C)CC4)cn3)ncc2F)sc2c(C(C)C)cn(C)c(=O)c12]

### Form A of Compound A Free Base

Table 1, supra, is reproduced below and sets forth the X-ray diffraction peaks observed for Form A of compound A free base.

TABLE 1
XRPD Peak Positions for Form A of Compound A Free Base

| Position (°2θ) | Intensity cps° |
|---|---|
| 9.0 | 1057 |
| 11.5 | 2282 |
| 12.9 | 1758 |
| 16.8 | 1433 |
| 17.9 | 1752 |
| 21.2 | 1097 |
| 22.0 | 443 |
| 23.5 | 1823 |
| 25.7 | 936 |
| 28.2 | 1093 |

FIG. 1 depicts an XRPD pattern of Form A of compound A free base.

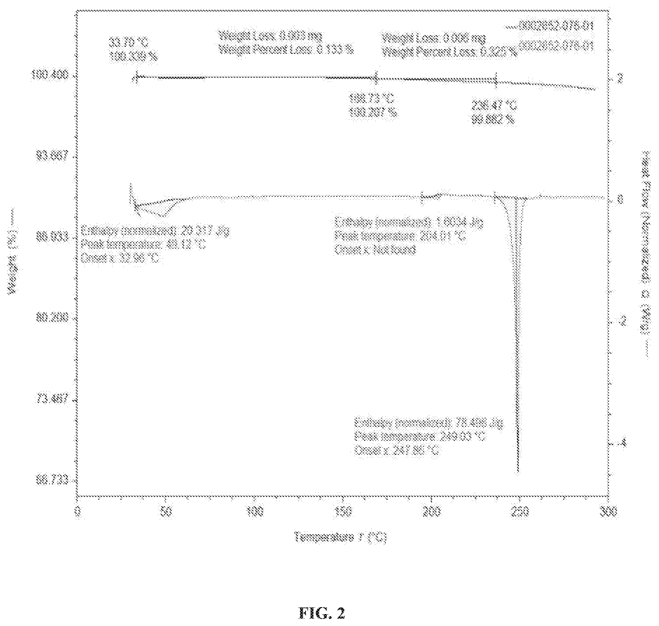

FIG. 2 depicts a DSC thermogram and TGA trace of Form A of compound A free base.

Properties of Form A of compound A free base are summarized below in Table 11.

TABLE 11
Properties of Form A of compound A Free Base

| Characterization | Instruments | Results |
|---|---|---|
| Crystallinity | XRPD | Moderate crystallinity |
| Thermal Weight Loss | TGA | 0.5% at 236.4° C. |
| Melting Point | DSC | Melting points at about |
| | | 189.87° C. and 247.86° C. |
| Morphology | PLM | Finely granular |
| Residual Solvent | NMR | No residual solvent |
| Purity | HPLC | 97.0% |

### Example 2—Form B of Compound A Free Base

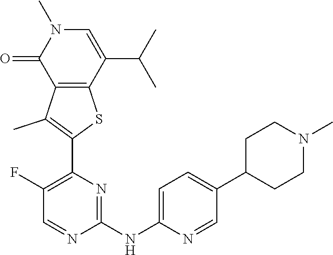 [SMILES: Cc1c(-c2nc(Nc3ccc(C4CCN(C)CC4)cn3)ncc2F)sc2c(C(C)C)cn(C)c(=O)c12]

### Form B of Compound A Free Base

Table 2, supra, is reproduced below and sets forth the X-ray diffraction peaks observed for Form B of compound A free base.

TABLE 2
XRPD Peak Positions for Form B of Compound A Free Base

| Position (°2θ) | Intensity cps° |
|---|---|
| 11.2 | 1838 |
| 16.0 | 1234 |
| 17.8 | 2637 |
| 17.9 | 634 |
| 18.3 | 3016 |
| 19.1 | 640 |
| 20.8 | 5682 |
| 22.5 | 874 |
| 22.9 | 1368 |
| 31.6 | 1095 |

FIG. 3 depicts an XRPD pattern of Form B of compound A free base.

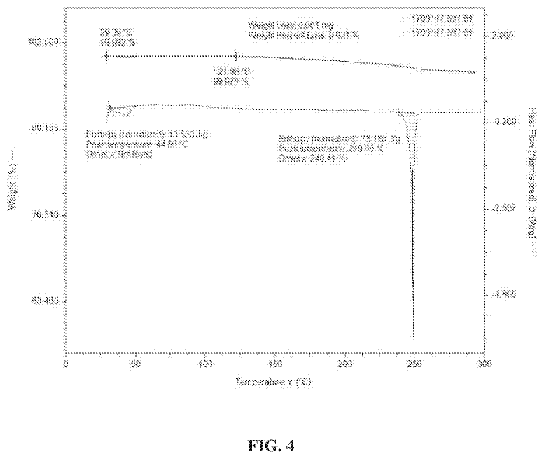

FIG. 4 depicts a DSC thermogram and TGA trace of Form B of compound A free base.

Properties of Form B of compound A free base are summarized below in Table 12.

TABLE 12
Properties of Form B of compound A Free Base

| Characterization | Instruments | Results |
|---|---|---|
| Crystallinity | | |
| Thermal Weight Loss | TGA | No obvious weight loss |
| Melting Point | DSC | Melting points at about |
| | | 248.41° C. |
| Morphology | PLM | Finely granular |
| ¹HNMR | NMR | Confirm its structure |
| | | No residual solvent |
| Purity | HPLC | 98.6% |

### Example 3—Form C of Compound A Free Base

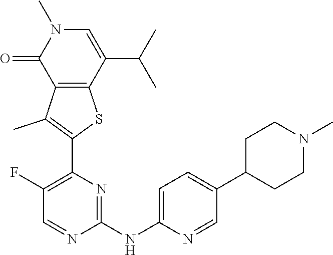 [SMILES: Cc1c(-c2nc(Nc3ccc(C4CCN(C)CC4)cn3)ncc2F)sc2c(C(C)C)cn(C)c(=O)c12]

### Form C of Compound A Free Base

Table 3, supra, is reproduced below and sets forth the X-ray diffraction peaks observed for Form C of compound A free base.

TABLE 3
XRPD Peak Positions for Form C of Compound A Free Base

| Position (°2θ) | Intensity cps° |
|---|---|
| 13.6 | 1965 |
| 16.4 | 2483 |
| 17.2 | 1233 |
| 20.0 | 1322 |
| 20.6 | 1123 |
| 20.8 | 1744 |
| 22.6 | 974 |
| 22.9 | 2889 |
| 23.7 | 1384 |
| 31.6 | 2668 |

FIG. 5 depicts an XRPD pattern of Form C of compound A free base.

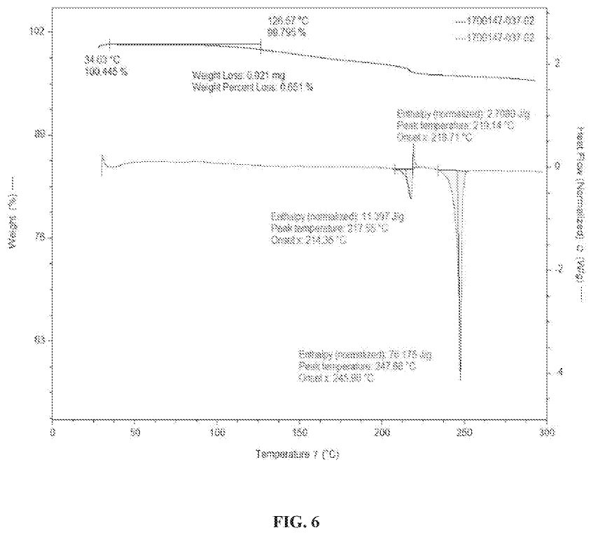

FIG. 6 depicts a DSC thermogram and TGA trace of Form C of compound A free base.

Properties of Form C of compound A free base are summarized below in Table 13.

TABLE 13
Properties of Form C of compound A Free Base

| Characterization | Instruments | Results |
|---|---|---|
| Crystallinity | XRPD | High crystallinity |
| Thermal Weight | TGA | 0.65% at 126.6° C. |
| Loss | | |
| Melting Point | DSC | Melting points at about |
| | | 214.35° C. and 245.98° C. |
| Morphology | PLM | Finely granular |
| ¹HNMR | NMR | Confirm its structure |
| | | Residue solvent of |
| | | DCM was 1.4% |
| Purity | HPLC | 98.0% |

### Example 4—Form A of Compound A Succinate Salt

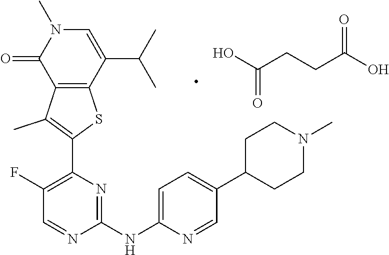

### Form A of Compound A Succinate Salt

Form A of compound A succinate salt was prepared similar to Examples 12-41, infra.

Three endothermic peaks (40.4° C., 113.1° C. and 148.6° C.) were checked by DSC. Form A of compound A succinate salt had a mass loss of 2.7% when heated to 105.4° C. by TGA. Residue solvent of EtOH was 0.19% by ¹H NMR.

Table 4, supra, is reproduced below and sets forth the X-ray diffraction peaks observed for Form A of compound A succinate salt.

TABLE 4
XRPD Peak Positions for Form A of Compound A Succinate Salt

| Position (°2θ) | Intensity cps° |
|---|---|
| 8.1 | 1598 |
| 10.8 | 1007 |
| 11.0 | 741 |
| 11.6 | 3424 |
| 12.0 | 556 |
| 13.6 | 860 |
| 15.8 | 640 |
| 17.5 | 872 |
| 24.4 | 1148 |
| 25.2 | 1001 |

FIG. 7 depicts an XRPD pattern of Form A of compound A succinate salt.

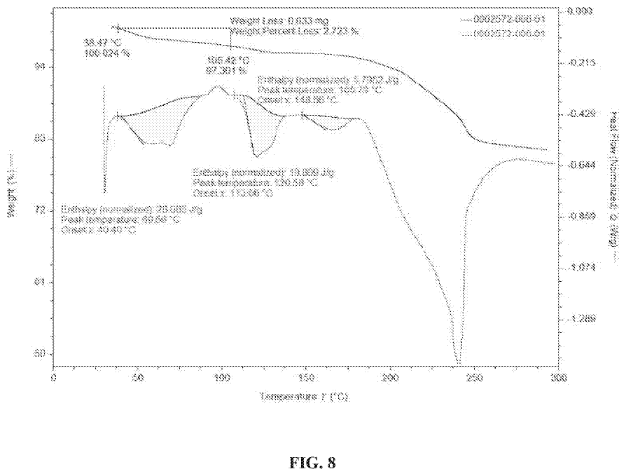

FIG. 8 depicts a DSC thermogram and TGA trace of Form A of compound A succinate salt.

### Example 5—Form B of Compound A Succinate Salt

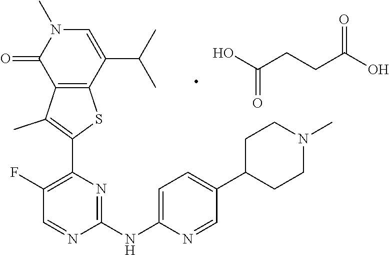

### Form B of Compound A Succinate Salt

Form B of compound A succinate salt was prepared similar to Examples 12-41, infra.

Form B of compound A succinate salt was obtained by stirring in 2-MeTHF at 25° C. Two endothermic peak (39.2° C. and 136.6° C.) were checked by DSC. Form B of compound A succinate salt had a mass loss of 2.4% when heated to 117.5° C. by TGA. Residue solvent of 2-MeTHF was 0.11% by ¹H NMR.

Table 5, supra, is reproduced below and sets forth the X-ray diffraction peaks observed for Form B of compound A succinate salt.

TABLE 5
XRPD Peak Positions for Form B of Compound A Succinate Salt

| Position (°2θ) | Intensity cps° |
|---|---|
| 11.9 | 1476 |
| 12.3 | 1571 |
| 14.3 | 388 |
| 15.1 | 786 |
| 18.6 | 2087 |
| 20.61 | 411 |
| 21.8 | 629 |
| 24.1 | 1904 |
| 24.6 | 2561 |
| 25.6 | 715 |

FIG. 9 depicts an XRPD pattern of Form B of compound A succinate salt.

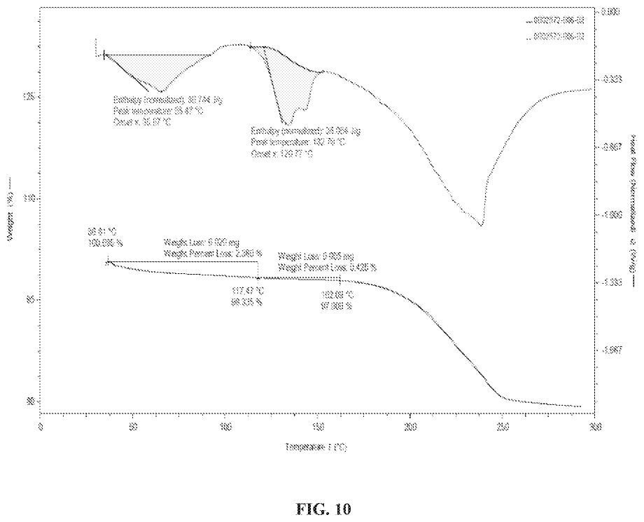

FIG. 10 depicts a DSC thermogram and TGA trace of Form B of compound A succinate salt.

### Example 6—Form C of Compound A Succinate Salt

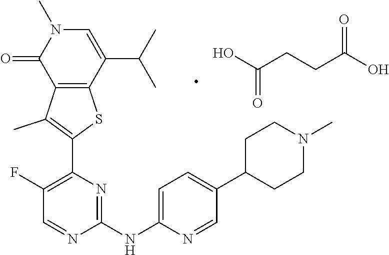

### Form C of Compound A Succinate Salt

Form C of compound A succinate salt was prepared similar to Examples 12-41, infra.

Form C of compound A succinate salt was obtained by stirring in almost solvent system at 25° C. and 50° C. One endothermic peak (144.0° C.) was checked by DSC. Form C of compound A succinate salt had a mass loss of 1.3% when heated to 105.6° C. by TGA. Residue solvent of acetone was 0.34% by ¹H NMR.

Table 6, supra, is reproduced below and sets forth the X-ray diffraction peaks observed for Form C of compound A succinate salt.

TABLE 6
XRPD Peak Positions for Form C of Compound A Succinate Salt

| Position (°2θ) | Intensity cps° |
|---|---|
| 9.2 | 3483 |
| 11.0 | 1956 |
| 11.9 | 3746 |
| 14.2 | 2881 |
| 15.1 | 3132 |
| 18.6 | 3295 |
| 18.9 | 3877 |
| 23.9 | 1819 |
| 24.0 | 2111 |
| 24.2 | 3363 |

FIG. 11 depicts an XRPD pattern of Form C of compound A succinate salt.

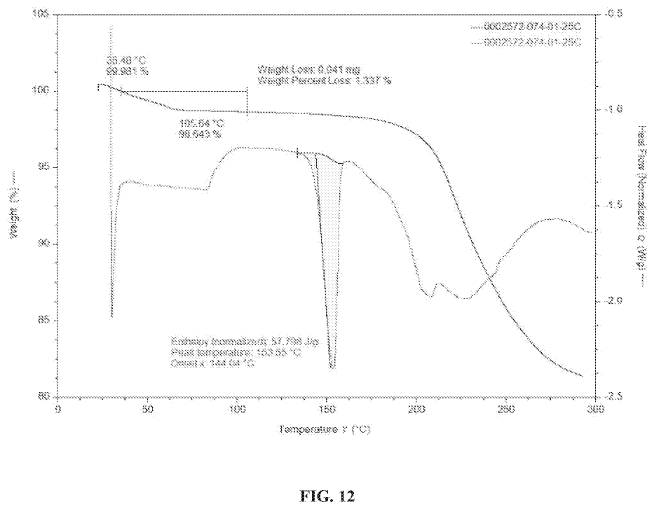

FIG. 12 depicts a DSC thermogram and TGA trace of Form C of compound A succinate salt.

### Example 7—Form D of Compound A Succinate Salt

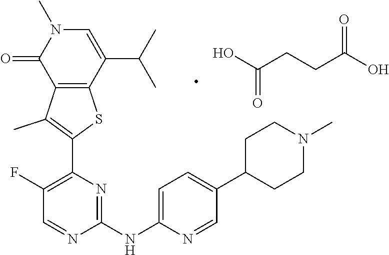

### Form D of Compound A Succinate Salt

Form D of compound A succinate salt was prepared similar to Examples 12-41, infra.

Form D of compound A succinate salt was obtained by stirring in THF/H₂O or acetone/H₂O systems at 25° C. and 50° C. One endothermic peak (138.7° C.) was checked by DSC. Form D of compound A succinate salt had a mass loss of 4.1% when heated to 131.2° C. by TGA. Residue solvent of THF was 0.9% by ¹H NMR.

Table 7, supra, is reproduced below and sets forth the X-ray diffraction peaks observed for Form D of compound A succinate salt.

TABLE 7
XRPD Peak Positions for Form D of Compound A Succinate Salt

| Position (°2θ) | Intensity cps° |
|---|---|
| 11.3 | 1074 |
| 12.3 | 2971 |
| 15.2 | 378 |
| 18.5 | 1582 |
| 19.4 | 2426 |
| 21.0 | 692 |
| 22.7 | 848 |
| 24.3 | 2178 |
| 24.7 | 2637 |
| 25.4 | 973 |

FIG. 13 depicts an XRPD pattern of Form D of compound A succinate salt.

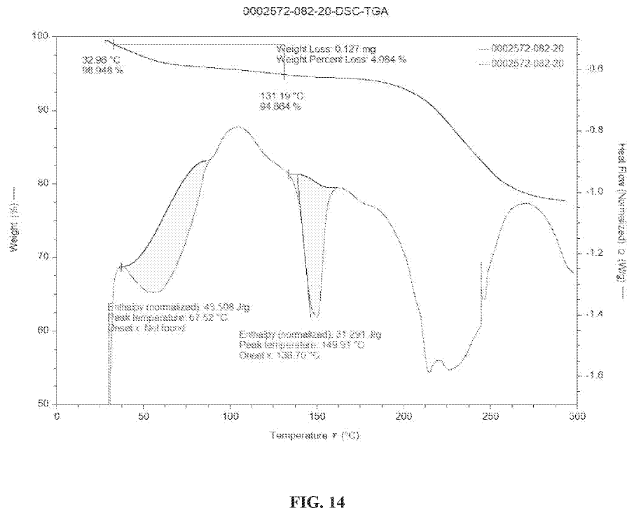

FIG. 14 depicts a DSC thermogram and TGA trace of Form D of compound A succinate salt.

### Example 8—Form E of Compound A Succinate Salt

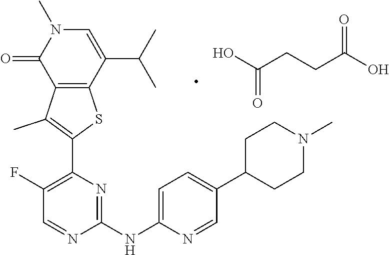

### Form E of Compound A Succinate Salt

Form E of compound A succinate salt was prepared similar to Examples 12-41, infra.

Form E of compound A succinate salt was obtained by stirring in MtBE at 50° C. or anti-solvent addition in MeOH systems with moderate crystallinity. One endothermic peak (134.1° C.) was checked by DSC. Form E of compound A succinate salt had a mass loss of 0.8% when heated to 125.4° C. by TGA. Residue solvent of MtBE was 0.8% by ¹HNMR.

Table 8, supra, is reproduced below and sets forth the X-ray diffraction peaks observed for Form E of compound A succinate salt.

TABLE 8
XRPD Peak Positions for Form E of Compound A Succinate Salt

| Position (°2θ) | Intensity cps° |
|---|---|
| 9.2 | 773 |
| 11.0 | 285 |
| 11.9 | 2806 |
| 14.2 | 652 |
| 15.1 | 1170 |
| 18.0 | 1309 |
| 18.6 | 993 |
| 18.9 | 1059 |
| 23.1 | 1781 |
| 24.1 | 2603 |

FIG. 15 depicts an XRPD pattern of Form E of compound A succinate salt.

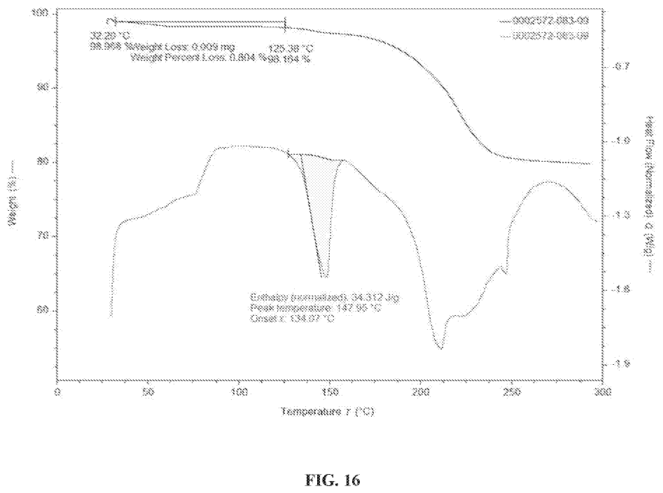

FIG. 16 depicts a DSC thermogram and TGA trace of Form E of compound A succinate salt.

### General Procedures for Examples 9, 10 and 11

X-Ray Powder Diffraction (XRPD) Data Collection Strategy: XRPD patterns of samples were recorded at room temperature on Aeris X-ray diffractometer (Almelo, The Netherlands) using Cu Kα radiation (λ=1.54 Å) at 40 kV, 15 mA passing through a Ni monochromater. Data was collected in a continuous scan mode with a step size of 0.02° and dwell time of 149 s over an angular range of 3° to 40° 20. The sample was loaded on a zero background holder and gently pressed by a clean glass slide to ensure coplanarity of the sample surface with the surface of the holder. Obtained diffractograms were analyzed and plotted with HighScore Plus software (V 5.0; Almelo, The Netherlands).

Thermogravimetric Analysis (TGA): TGA was performed using a Discovery TGA 5500 (TAR Instruments, New Castle, Delaware, USA) instrument operating with TRIOS software (Version 5.0). The sample was placed in an aluminum pan. The sample cell was purged with dry nitrogen at a flow rate of 15 mL/min. A heating rate of 10° C./min from 25-350° C. was used in all the experiments.

Differential Scanning calorimetry (DSC): Conventional DSC experiments were performed by using either Q100 or Discovery DSC 2500 (TAR Instruments, New Castle, Delaware, USA) instrument equipped with a refrigerated cooling system (RCS90). The sample cell was purged with dry nitrogen at a flow rate of 50 mL/min. Accurately weighed samples (2-5 mg) placed in TZero pans with a pin hole were scanned at a heating rate of 10° C./min over a desired temperature range.

Examples 9 and 10 were prepared by the reactive crystallization procedures described below, e.g., method I, method II and method III.

Method-I: Compound A (ca. 50 mg) and succinic acid (ca. 13 mg) were suspended in solvent (ca. 1-2 mL) and stirred at ambient temperature for ˜15 hours. The resultant solids were collected by filtration, and then characterized.

Method-II: Compound A (ca. 50 mg) and succinic acid (ca. 13 mg) together were dissolve in minimum amount of the stated solvent while heating at 80° C. When necessary, the hot solutions were filtered using a 0.45 μm PTFE syringe filter. The resultant solutions were kept at RT to induce precipitation of the solids.

Method-III: In a separate vial, 235 mg of succinic acid was dissolved in 20 mL of ethanol and used as a stock solution. Compound A (ca. 50 mg) was dissolved in minimum amount of solvent. One equivalent of succinic acid containing EtOH solution was added. The resultant solids were collected by filtration and characterized.

### Example 9—Form F of Compound A Succinate Salt

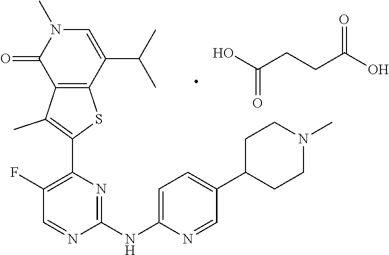

### Form F of Compound A Succinate Salt

Table 9, supra, is reproduced below and sets forth the X-ray diffraction peaks observed for Form F of compound A succinate salt.

TABLE 9
XRPD Peak Positions for Form F of Compound A Succinate Salt

| Position (°2θ) | Rel. Int. [%] |
|---|---|
| 6.0 | 69.55 |
| 6.5 | 34.06 |
| 8.1 | 11.08 |
| 9.8 | 19.53 |
| 11.6 | 18.09 |
| 12.5 | 100.00 |
| 13.5 | 9.30 |
| 14.8 | 10.73 |
| 15.8 | 40.61 |
| 16.4 | 16.76 |
| 17.2 | 18.19 |
| 18.4 | 40.48 |
| 18.6 | 37.40 |
| 19.1 | 31.21 |
| 20.2 | 9.75 |
| 21.2 | 36.05 |
| 22.4 | 7.29 |
| 23.7 | 17.44 |
| 24.5 | 90.59 |
| 24.7 | 90.50 |
| 26.3 | 15.31 |
| 27.4 | 10.51 |
| 28.5 | 12.96 |
| 32.1 | 3.11 |
| 34.0 | 3.97 |
| 37.1 | 3.14 |

FIG. 17 depicts an XRPD pattern of Form F of compound A succinate salt.

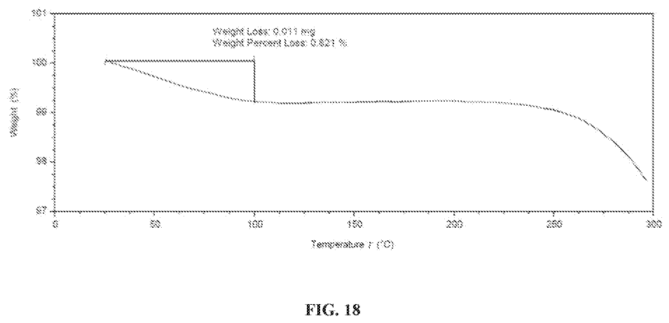

FIG. 18 depicts a TGA trace of Form F of compound A succinate salt.

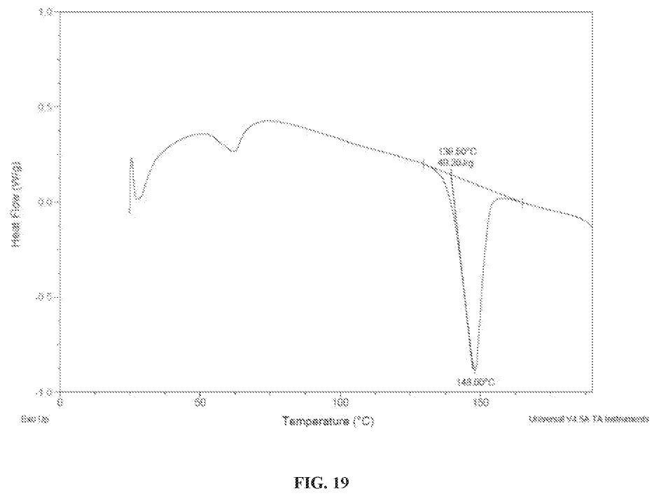

FIG. 19 depicts a DSC thermogram of Form F of compound A succinate salt.

### Example 10—Form G of Compound A Succinate Salt

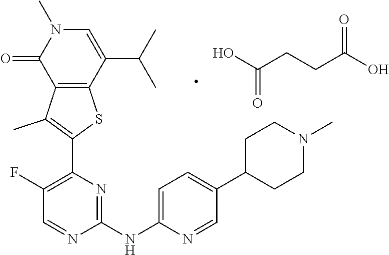

### Form G of Compound A Succinate Salt

Form G of compound A succinate salt was prepared by reactive crystallization experiments from 1:1 v/v CHCl₃—MeOH solution.

Table 10, supra, is reproduced below and sets forth the X-ray diffraction peaks observed for Form G of compound A succinate salt.

TABLE 10
XRPD Peak Positions for Form G of Compound A Succinate Salt

| Position (°2θ) | Rel. Int. [%] |
|---|---|
| 4.6 | 3.48 |
| 5.4 | 32.18 |
| 6.0 | 100.00 |
| 7.5 | 32.37 |
| 9.1 | 0.46 |
| 9.6 | 0.72 |
| 10.0 | 3.90 |
| 10.9 | 5.72 |
| 11.9 | 19.84 |
| 12.8 | 14.01 |
| 14.0 | 7.12 |
| 14.2 | 3.37 |
| 15.1 | 35.06 |
| 15.3 | 37.67 |
| 15.6 | 3.32 |
| 15.9 | 2.56 |
| 16.4 | 3.81 |
| 16.9 | 3.49 |
| 17.2 | 15.36 |
| 17.8 | 10.88 |
| 17.9 | 23.54 |
| 18.3 | 3.34 |
| 18.6 | 5.86 |
| 18.9 | 9.48 |
| 19.3 | 10.66 |
| 19.8 | 7.14 |
| 20.0 | 16.43 |
| 20.4 | 5.45 |
| 20.8 | 4.20 |
| 21.0 | 2.46 |
| 21.5 | 1.70 |
| 22.0 | 8.62 |
| 22.3 | 1.26 |
| 22.9 | 9.44 |
| 23.1 | 21.49 |
| 23.3 | 26.91 |
| 23.4 | 36.38 |
| 23.5 | 21.20 |
| 24.0 | 15.44 |
| 24.2 | 10.39 |
| 24.4 | 3.00 |
| 24.6 | 5.88 |
| 24.8 | 3.52 |
| 25.1 | 15.50 |
| 25.7 | 1.04 |
| 26.5 | 1.35 |
| 27.2 | 8.67 |
| 27.7 | 22.08 |
| 28.0 | 2.65 |
| 28.4 | 5.03 |
| 28.6 | 2.23 |
| 29.1 | 4.45 |
| 29.5 | 2.31 |
| 29.9 | 0.78 |
| 30.1 | 1.88 |
| 30.8 | 1.77 |
| 31.2 | 2.08 |
| 31.7 | 2.20 |
| 32.1 | 1.43 |
| 32.4 | 2.60 |
| 32.5 | 3.02 |
| 32.7 | 1.29 |
| 32.9 | 0.95 |
| 33.2 | 3.46 |
| 33.5 | 2.15 |
| 34.3 | 3.46 |
| 34.5 | 1.88 |
| 34.9 | 2.81 |
| 35.2 | 1.27 |
| 35.7 | 3.28 |
| 36.2 | 0.86 |
| 36.7 | 1.54 |
| 37.6 | 2.38 |
| 37.9 | 0.89 |
| 38.3 | 1.25 |
| 38.5 | 1.60 |
| 39.4 | 1.18 |

FIG. 20 depicts an XRPD pattern of Form G of compound A succinate salt.

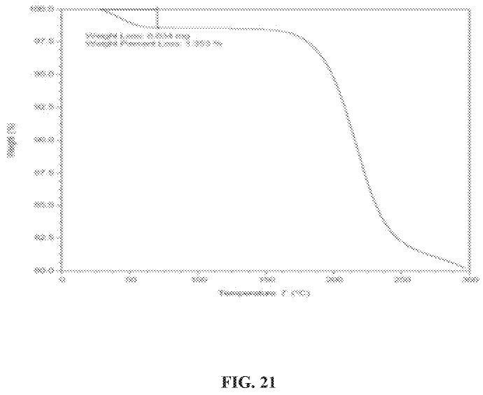

FIG. 21 depicts a TGA trace of Form G of compound A succinate salt.

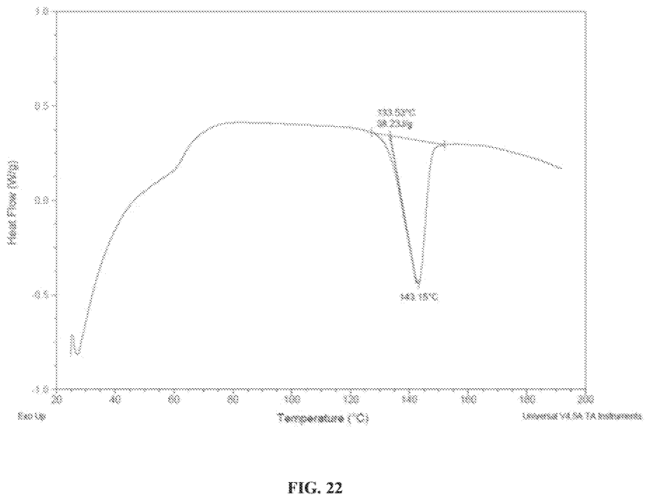

FIG. 22 depicts a DSC thermogram of Form G of compound A succinate salt.

### Example 11—Single Crystal of Form G of Compound A Succinate Salt

Structural elucidation by single crystal X-ray diffraction (SCXRD) revealed that Form G of Compound A succinate salt has one equivalent of CHCl₃ molecule per one molecule of Compound A. This data along with TGA data suggest that CHCl₃ plays a role in stabilizing the Form G crystal lattice.

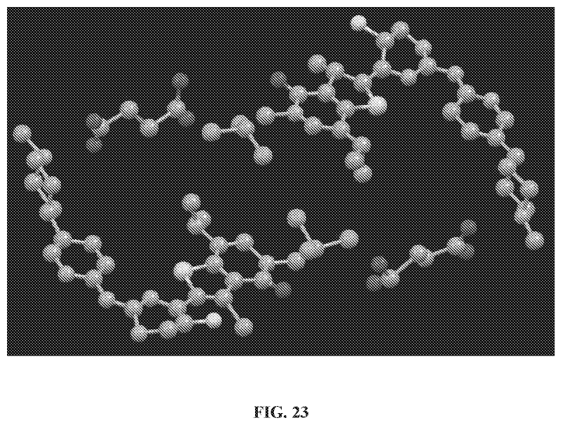

FIG. 23 depicts an X-ray diffraction pattern of a single crystal of Compound A succinate salt, Form G.

Examples 12-41 were prepared as follows:

Step (1): Compound A (˜30 mg) was added to a reaction vessel (RV1) and charged with solvent (1 mL). The mixture was stirred at room temperature to obtain a clear solution or suspension.

Step (2): Counter-ion (˜1.1 eq.) was charged with solvent (0.5 mL) into a separate reaction vessel (RV2) to obtain a clear solution or suspension. This solution or suspension was added dropwise into RV1 with stirring.

Step (3): The mixture in RV1 was heated and stirred at 50° C. for 2 hours and subsequently cooled down to 25° C. for 3 hours.

Step (4): The mixture was centrifuged, and the filtrate cake was dried. The remaining solution of the filtrate cake was evaporated at 25° C. under atmospheric pressure to obtain a solid product and characterized, infra.

### Example 12—Form A of Compound A Hydrochloride Salt

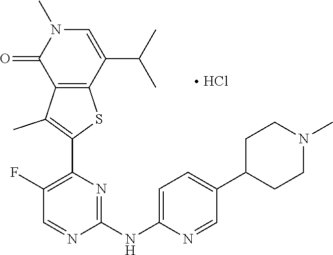 [SMILES: Cc1c(-c2nc(Nc3ccc(C4CCN(C)CC4)cn3)ncc2F)sc2c(C(C)C)cn(C)c(=O)c12 ; Cl]

### Form A of Compound A Hydrochloride Salt

Table 14 sets forth the X-ray diffraction peaks observed for Form A of compound A hydrochloride salt.

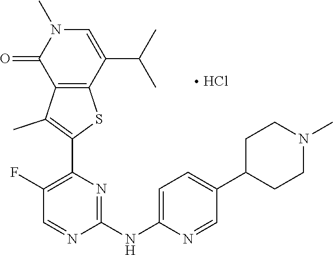 [SMILES: Cc1c(-c2nc(Nc3ccc(C4CCN(C)CC4)cn3)ncc2F)sc2c(C(C)C)cn(C)c(=O)c12 ; Cl]

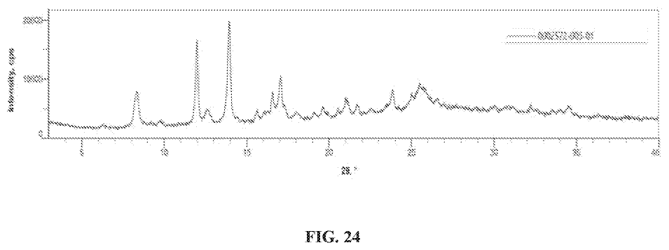

FIG. 24 depicts an XRPD pattern of Form A of compound A hydrochloride salt.

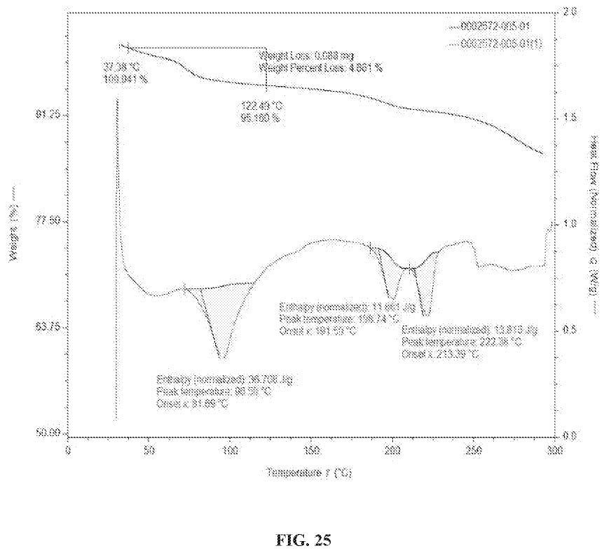

FIG. 25 depicts a DSC thermogram and TGA trace of Form A of compound A hydrochloride salt.

### Example 13—Form B of Compound A Hydrochloride Salt

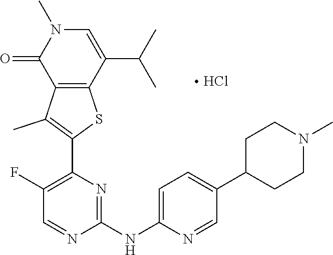 [SMILES: Cc1c(-c2nc(Nc3ccc(C4CCN(C)CC4)cn3)ncc2F)sc2c(C(C)C)cn(C)c(=O)c12 ; Cl]

### Form B of Compound A Hydrochloride Salt

Table 15 sets forth the X-ray diffraction peaks observed for Form B of compound A hydrochloride salt.

TABLE 15
XRPD Peak Positions for Form B of
Compound A Hydrochloride Salt

| Position (°2θ) | Intensity cps° |
|---|---|
| 9.3 | 232 |
| 11.7 | 596 |
| 12.1 | 312 |
| 16.2 | 564 |
| 22.2 | 401 |
| 23.1 | 561 |
| 25.6 | 489 |
| 25.7 | 552 |
| 26.4 | 807 |
| 32.9 | 297 |

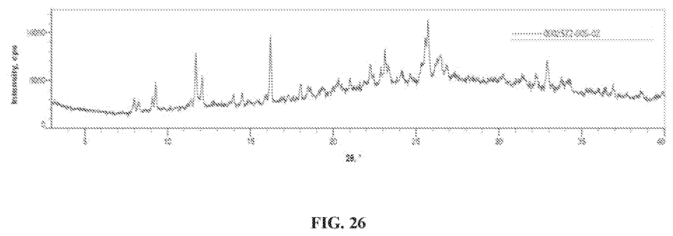

FIG. 26 depicts an XRPD pattern of Form B of compound A hydrochloride salt.

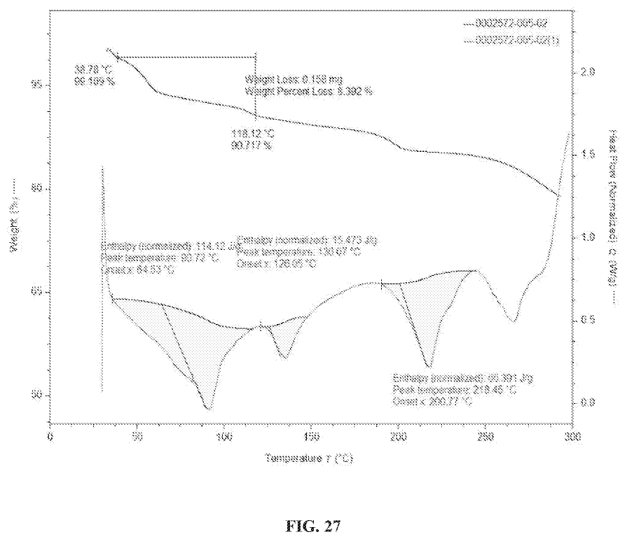

FIG. 27 depicts a DSC thermogram and TGA trace of Form B of compound A hydrochloride salt.

### Example 14—Form C of Compound A Hydrochloride Salt

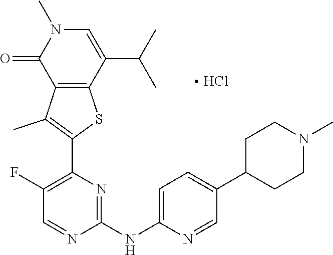 [SMILES: Cc1c(-c2nc(Nc3ccc(C4CCN(C)CC4)cn3)ncc2F)sc2c(C(C)C)cn(C)c(=O)c12 ; Cl]

### Form C of Compound A Hydrochloride Salt

Table 16 sets forth the X-ray diffraction peaks observed for Form C of compound A hydrochloride salt.

TABLE 16
XRPD Peak Positions for Form C of
Compound A Hydrochloride Salt

| Position (°2θ) | Intensity cps° |
|---|---|
| 8.2 | 495 |
| 10.3 | 684 |
| 11.7 | 303 |
| 13.4 | 385 |
| 19.4 | 479 |
| 24.3 | 888 |
| 25.2 | 1429 |

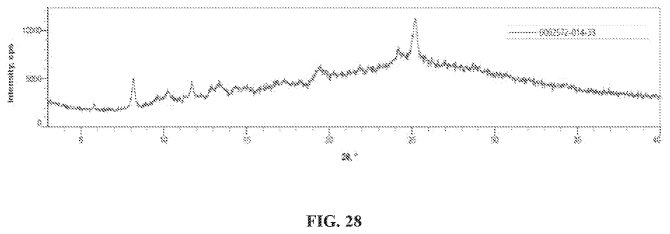

FIG. 28 depicts an XRPD pattern of Form C of compound A hydrochloride salt.

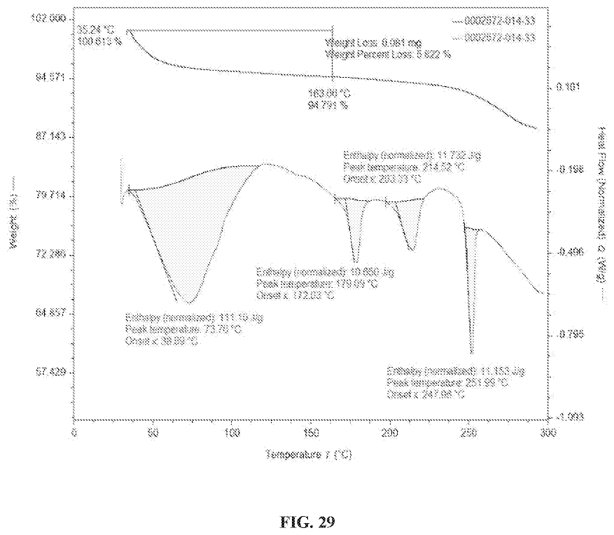

FIG. 29 depicts a DSC thermogram and TGA trace of Form C of compound A hydrochloride salt.

### Example 15—Form D of Compound A Hydrochloride Salt

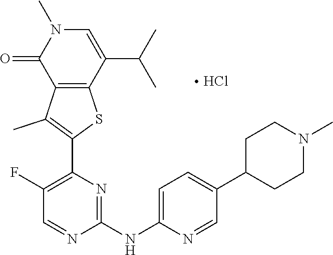 [SMILES: Cc1c(-c2nc(Nc3ccc(C4CCN(C)CC4)cn3)ncc2F)sc2c(C(C)C)cn(C)c(=O)c12 ; Cl]

### Form D of Compound A Hydrochloride Salt

Table 17 sets forth the X-ray diffraction peaks observed for Form D of compound A hydrochloride salt.

TABLE 17
XRPD Peak Positions for Form D of
Compound A Hydrochloride Salt

| Position (°2θ) | Intensity cps° |
|---|---|
| 5.5 | 556 |
| 7.9 | 238 |
| 9.3 | 62 |
| 9.6 | 403 |
| 11.0 | 2200 |
| 13.2 | 107 |
| 13.8 | 274 |
| 15.9 | 92 |
| 16.6 | 178 |
| 23.8 | 482 |

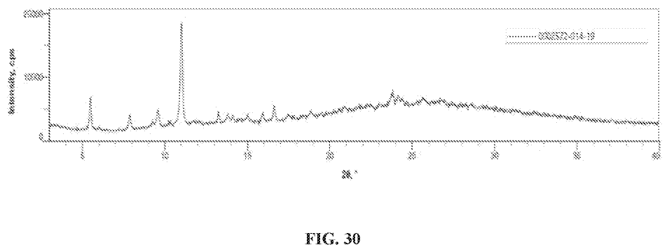

FIG. 30 depicts an XRPD pattern of Form D of compound A hydrochloride salt.

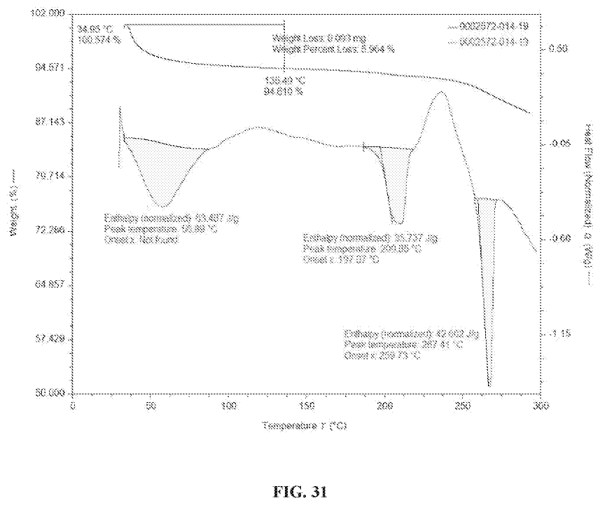

FIG. 31 depicts a DSC thermogram and TGA trace of Form D of compound A hydrochloride salt.

### Example 16—Form E of Compound A Hydrochloride Salt

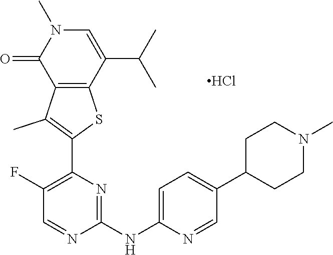 [SMILES: Cc1c(-c2nc(Nc3ccc(C4CCN(C)CC4)cn3)ncc2F)sc2c(C(C)C)cn(C)c(=O)c12 ; Cl]

### Form E of Compound A Hydrochloride Salt

Table 18 sets forth the X-ray diffraction peaks observed for Form E of compound A hydrochloride salt.

TABLE 18
XRPD Peak Positions for Form E of
Compound A Hydrochloride Salt

| Position (°2θ) | Intensity cps° |
|---|---|
| 12.0 | 3107 |
| 14.0 | 3451 |
| 15.6 | 437 |
| 16.6 | 708 |
| 17.1 | 990 |
| 17.3 | 789 |
| 21.1 | 622 |
| 23.8 | 1047 |
| 25.6 | 1088 |
| 26.6 | 472 |

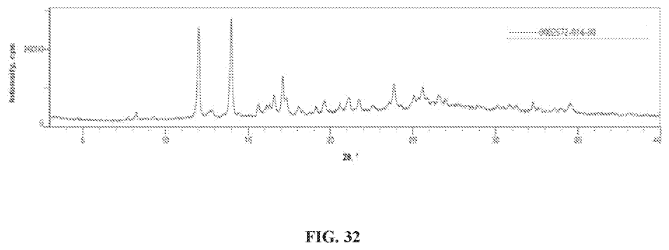

FIG. 32 depicts an XRPD pattern of Form E of compound A hydrochloride salt.

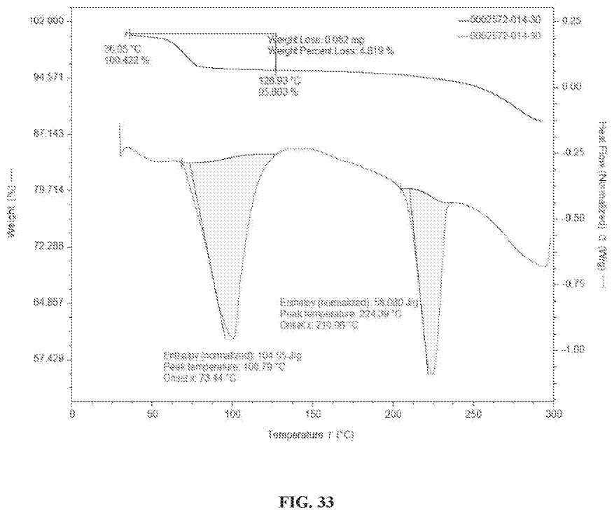

FIG. 33 depicts a DSC thermogram and TGA trace of Form E of compound A hydrochloride salt.

### Example 17—Form F of Compound A Hydrochloride Salt

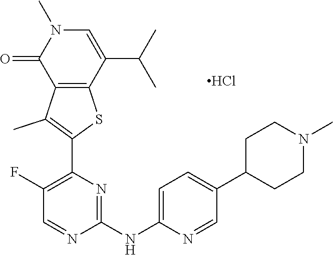 [SMILES: Cc1c(-c2nc(Nc3ccc(C4CCN(C)CC4)cn3)ncc2F)sc2c(C(C)C)cn(C)c(=O)c12 ; Cl]

### Form F of Compound A Hydrochloride Salt

Table 19 sets forth the X-ray diffraction peaks observed for Form F of compound A hydrochloride salt.

TABLE 19
XRPD Peak Positions for Form F of
Compound A Hydrochloride Salt

| Position (°2θ) | Intensity cps° |
|---|---|
| 8.4 | 599 |
| 9.7 | 299 |
| 12.3 | 2072 |
| 15.6 | 487 |
| 17.6 | 481 |
| 20.2 | 728 |
| 23.6 | 612 |
| 25.0 | 1078 |
| 26.1 | 271 |
| 34.7 | 151 |

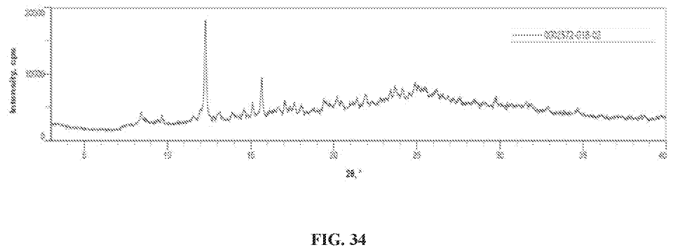

FIG. 34 depicts an XRPD pattern of Form F of compound A hydrochloride salt.

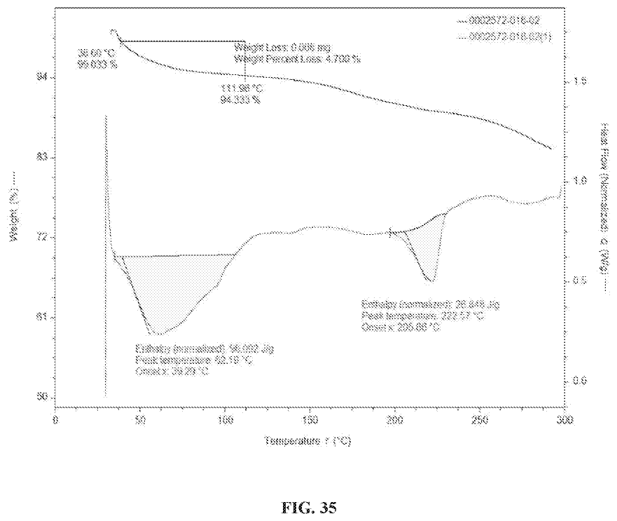

FIG. 35 depicts a DSC thermogram and TGA trace of Form F of compound A hydrochloride salt.

### Example 18—Form A of Compound A Maleate Salt

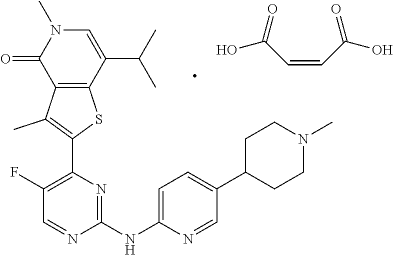 [SMILES: Cc1c(-c2nc(Nc3ccc(C4CCN(C)CC4)cn3)ncc2F)sc2c(C(C)C)cn(C)c(=O)c12 ; O=C(O)/C=C\C(=O)O]

### Form A of Compound A Maleate Salt

Table 20 sets forth the X-ray diffraction peaks observed for Form A of compound A maleate salt.

TABLE 20
XRPD Peak Positions for Form A of Compound A Maleate Salt

| Position (°2θ) | Intensity cps° |
|---|---|
| 12.2 | 2285 |
| 17.2 | 1123 |
| 17.7 | 1056 |
| 18.4 | 888 |
| 21.1 | 1206 |
| 22.2 | 1216 |
| 22.8 | 1068 |
| 24.5 | 2972 |
| 26.0 | 1122 |
| 31.6 | 2362 |

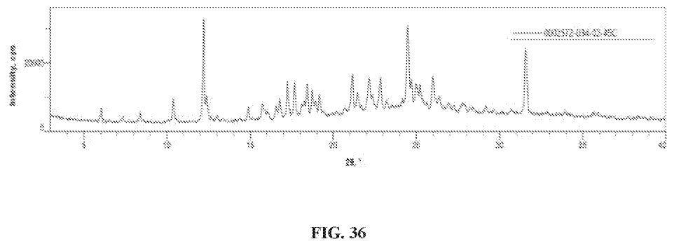

FIG. 36 depicts an XRPD pattern of Form A of compound A maleate salt.

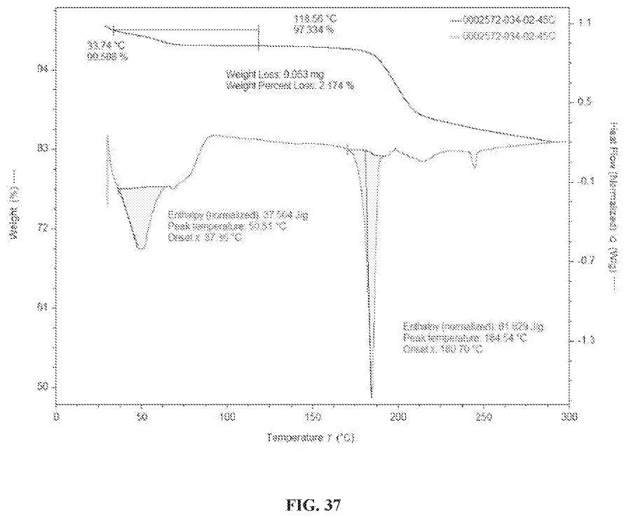

FIG. 37 depicts a DSC thermogram and TGA trace of Form A of compound A maleate salt.

### Example 19—Form A of Compound A Sulfate Salt

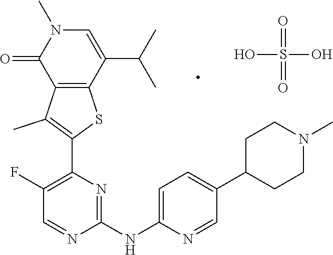

### Form A of Compound A Sulfate Salt

Table 21 sets forth the X-ray diffraction peaks observed for Form A of compound A sulfate salt.

TABLE 21
XRPD Peak Positions for Form A of Compound A Sulfate Salt

| Position (°2θ) | Intensity cps° |
|---|---|
| 4.7 | 95 |
| 8.0 | 1131 |
| 11.7 | 508 |
| 14.7 | 770 |
| 17.6 | 166 |
| 20.2 | 953 |
| 24.7 | 613 |
| 26.4 | 2418 |

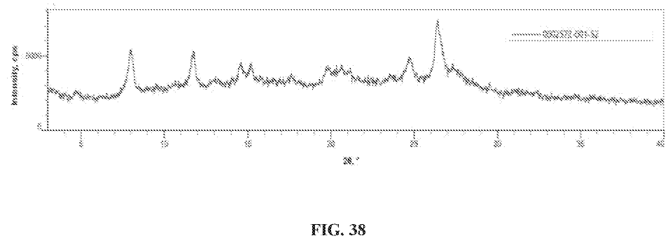

FIG. 38 depicts an XRPD pattern of Form A of compound A sulfate salt.

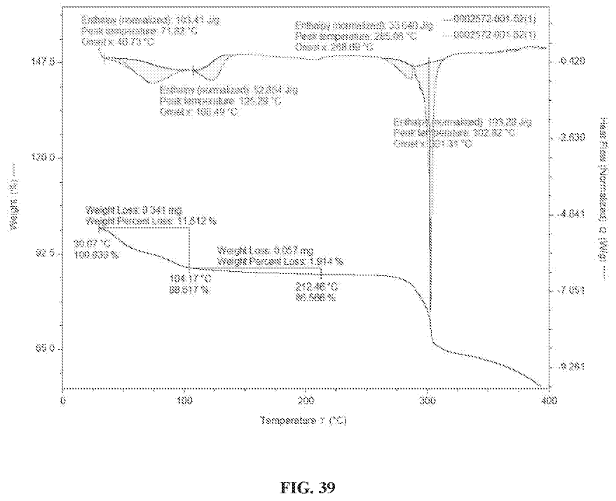

FIG. 39 depicts a DSC thermogram and TGA trace of Form A of compound A sulfate salt.

### Example 20—Form B of Compound A Sulfate Salt

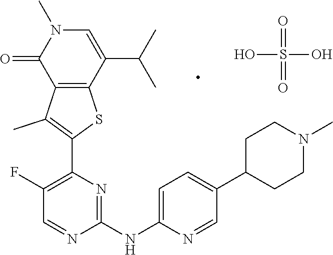

### Form B of Compound A Sulfate Salt

Table 22 sets forth the X-ray diffraction peaks observed for Form B of compound A sulfate salt.

TABLE 22
XRPD Peak Positions for Form B of Compound A Sulfate Salt

| Position (°2θ) | Intensity cps° |
|---|---|
| 12.0 | 3693 |
| 13.6 | 272 |
| 13.9 | 1171 |
| 15.6 | 656 |
| 18.9 | 396 |
| 22.5 | 370 |
| 23.8 | 583 |
| 31.7 | 1010 |
| 32.0 | 572 |
| 33.7 | 1109 |

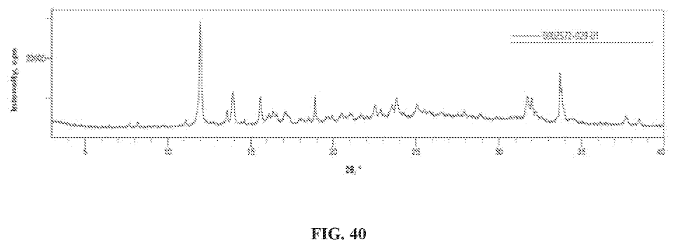

FIG. 40 depicts an XRPD pattern of Form B of compound A sulfate salt.

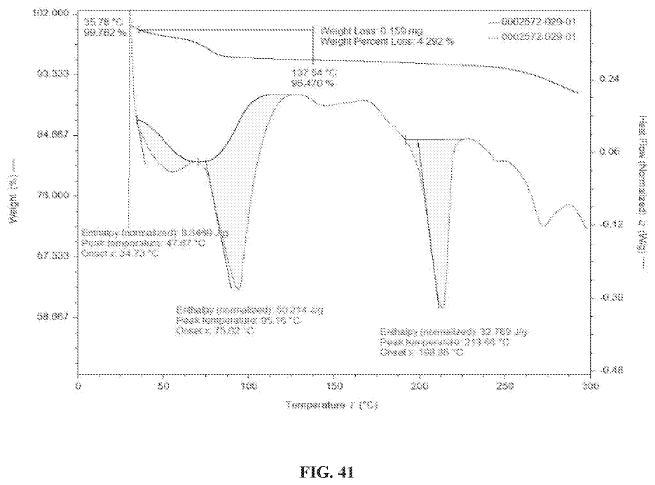

FIG. 41 depicts a DSC thermogram and TGA trace of Form B of compound A sulfate salt.

### Example 21—Form C of Compound A Sulfate Salt

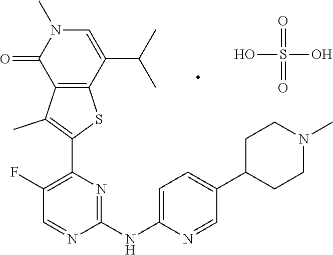

### Form C of Compound A Sulfate Salt

Table 23 sets forth the X-ray diffraction peaks observed for Form C of compound A sulfate salt.

TABLE 23
XRPD Peak Positions for Form C of Compound A Sulfate Salt

| Position (°2θ) | Intensity cps° |
|---|---|
| 7.7 | 202 |
| 8.1 | 444 |
| 9.1 | 421 |
| 11.4 | 211 |
| 11.8 | 377 |
| 15.7 | 496 |
| 18.9 | 176 |
| 25.3 | 531 |
| 25.9 | 629 |
| 33.7 | 453 |

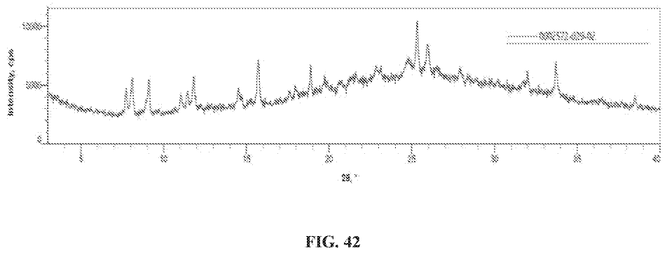

FIG. 42 depicts an XRPD pattern of Form C of compound A sulfate salt.

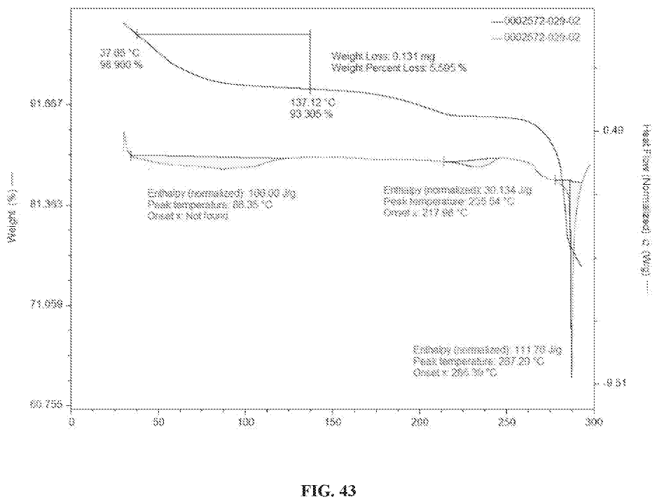

FIG. 43 depicts a DSC thermogram and TGA trace of Form C of compound A sulfate salt.

### Example 22—Form A of Compound A Phosphate Salt

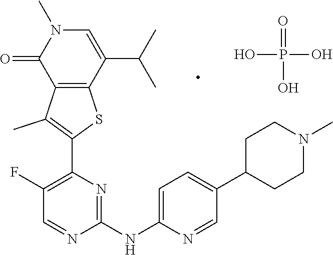

### Form A of Compound A Phosphate Salt

Table 24 sets forth the X-ray diffraction peaks observed for Form A of compound A phosphate salt.

TABLE 24
XRPD Peak Positions for Form A of Compound A Phosphate Salt

| Position (°2θ) | Intensity cps° |
|---|---|
| 9.4 | 1620 |
| 11.1 | 125 |
| 13.3 | 766 |
| 19.0 | 695 |
| 22.0 | 330 |
| 24.6 | 3654 |
| 27.3 | 533 |

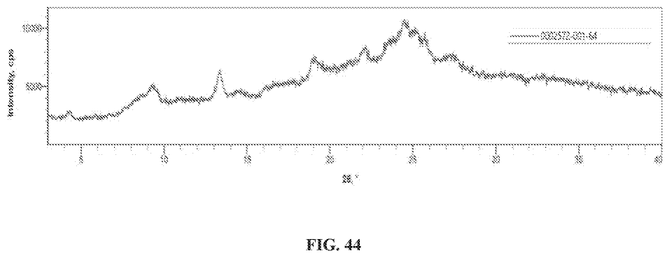

FIG. 44 depicts an XRPD pattern of Form A of compound A phosphate salt.

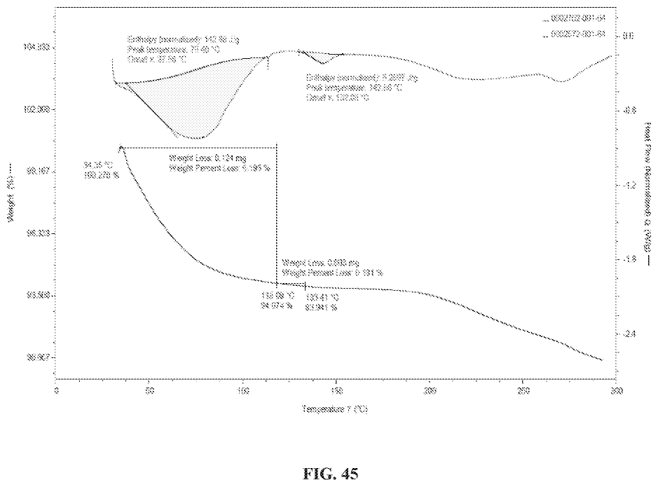

FIG. 45 depicts a DSC thermogram and TGA trace of Form A of compound A phosphate salt.

### Example 23—Form A of Compound A L-Tartrate Salt

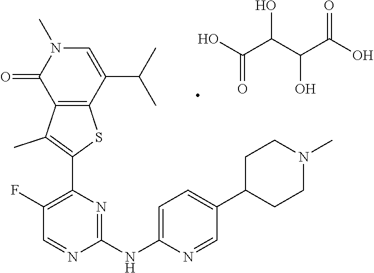 [SMILES: Cc1c(-c2nc(Nc3ccc(C4CCN(C)CC4)cn3)ncc2F)sc2c(C(C)C)cn(C)c(=O)c12 ; O=C(O)C(O)C(O)C(=O)O]

### Form A of Compound A L-Tartrate Salt

Table 25 sets forth the X-ray diffraction peaks observed for Form A of compound A L-tartrate salt.

TABLE 25
XRPD Peak Positions for Form A of Compound A L-Tartrate Salt

| Position (°2θ) | Intensity cps° |
|---|---|
| 4.6 | 311 |
| 4.9 | 673 |
| 8.3 | 528 |
| 8.7 | 2198 |
| 9.4 | 908 |
| 11.6 | 809 |
| 15.0 | 756 |
| 20.6 | 258 |
| 24.1 | 926 |
| 27.1 | 961 |

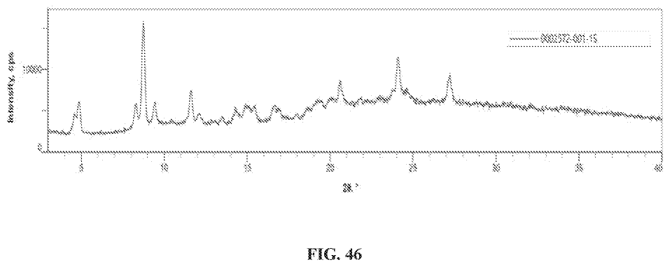

FIG. 46 depicts an XRPD pattern of Form A of compound A L-tartrate salt.

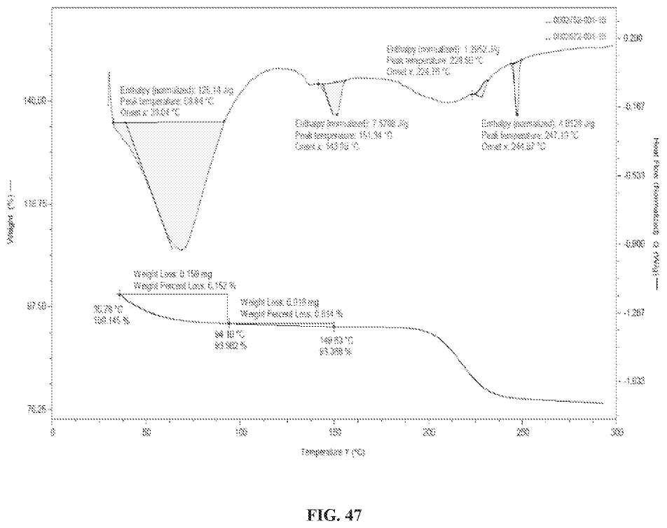

FIG. 47 depicts a DSC thermogram and TGA trace of Form A of compound A L-tartrate salt.

### Example 24—Form A of Compound A Fumarate Salt

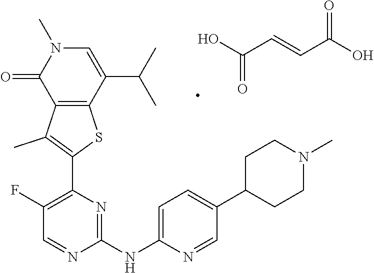 [SMILES: Cc1c(-c2nc(Nc3ccc(C4CCN(C)CC4)cn3)ncc2F)sc2c(C(C)C)cn(C)c(=O)c12 ; O=C(O)/C=C/C(=O)O]

### Form A of Compound A Fumarate Salt

Table 26 sets forth the X-ray diffraction peaks observed for Form A of compound A fumarate salt.

TABLE 26
XRPD Peak Positions for Form A of Compound A Fumarate Salt

| Position (°2θ) | Intensity cps° |
|---|---|
| 5.6 | 2046 |
| 7.9 | 1823 |
| 10.8 | 303 |
| 11.5 | 3759 |
| 13.4 | 897 |
| 17.2 | 910 |
| 19.3 | 745 |
| 24.3 | 2241 |
| 25.2 | 781 |
| 26.0 | 1330 |

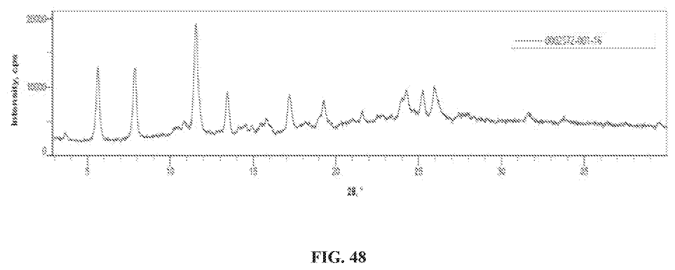

FIG. 48 depicts an XRPD pattern of Form A of compound A fumarate salt.

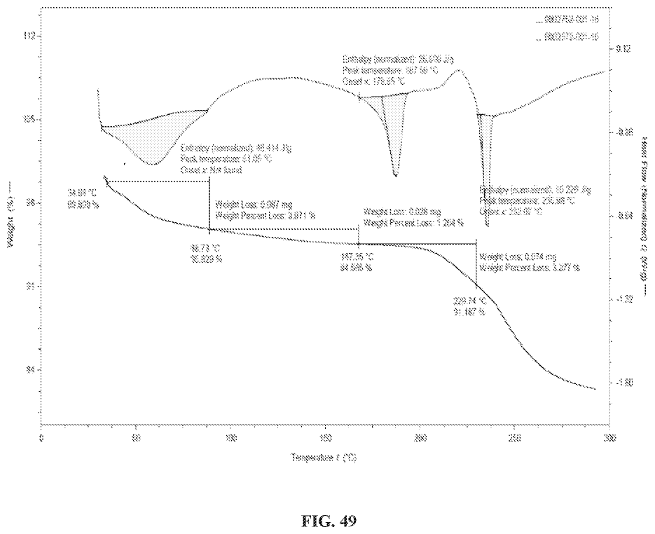

FIG. 49 depicts a DSC thermogram and TGA trace of Form A of compound A fumarate salt.

### Example 25—Form B of Compound A Fumarate Salt

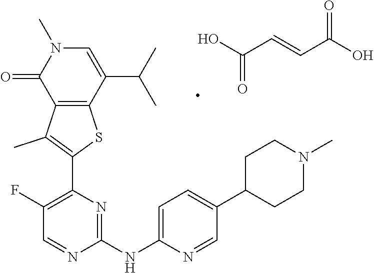 [SMILES: Cc1c(-c2nc(Nc3ccc(C4CCN(C)CC4)cn3)ncc2F)sc2c(C(C)C)cn(C)c(=O)c12 ; O=C(O)/C=C/C(=O)O]

### Form B of Compound A Fumarate Salt

Table 27 sets forth the X-ray diffraction peaks observed for Form B of compound A fumarate salt.

TABLE 27
XRPD Peak Positions for Form B of Compound A Fumarate Salt

| Position (°2θ) | Intensity cps° |
|---|---|
| 5.3 | 2061 |
| 5.8 | 954 |
| 9.0 | 770 |
| 10.9 | 549 |
| 11.9 | 2525 |
| 18.1 | 1207 |
| 18.6 | 420 |
| 20.3 | 734 |
| 23.6 | 3515 |
| 24.3 | 602 |

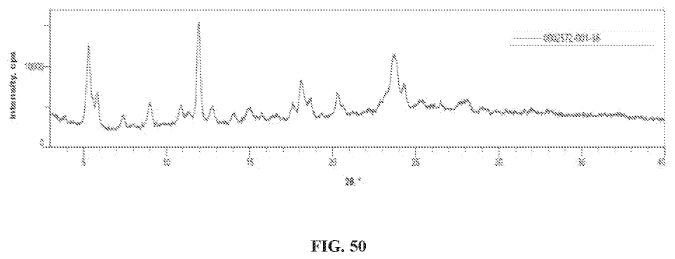

FIG. 50 depicts an XRPD pattern of Form B of compound A fumarate salt.

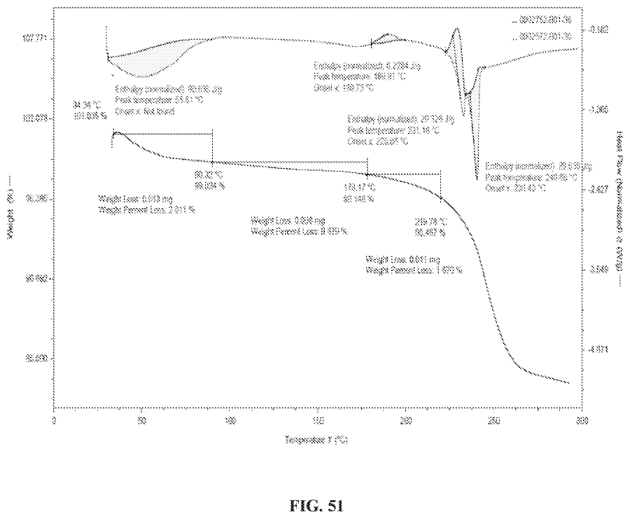

FIG. 51 depicts a DSC thermogram and TGA trace of Form B of compound A fumarate salt.

### Example 26—Form C of Compound A Fumarate Salt

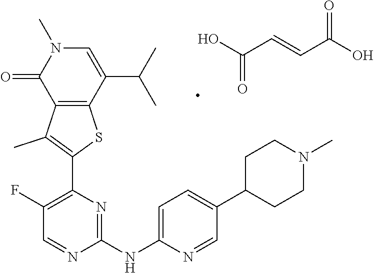 [SMILES: Cc1c(-c2nc(Nc3ccc(C4CCN(C)CC4)cn3)ncc2F)sc2c(C(C)C)cn(C)c(=O)c12 ; O=C(O)/C=C/C(=O)O]

### Form C of Compound A Fumarate Salt

Table 28 sets forth the X-ray diffraction peaks observed for Form C of compound A fumarate salt.

TABLE 28
XRPD Peak Positions for Form C of Compound A Fumarate Salt

| Position (°2θ) | Intensity cps° |
|---|---|
| 5.3 | 638 |
| 5.8 | 1250 |
| 7.5 | 1137 |
| 9.6 | 2075 |
| 10.4 | 2093 |
| 12.1 | 1777 |
| 14.7 | 686 |
| 16.1 | 536 |
| 22.4 | 936 |
| 24.5 | 1674 |

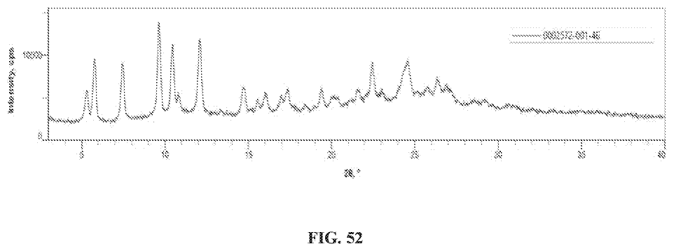

FIG. 52 depicts an XRPD pattern of Form C of compound A fumarate salt.

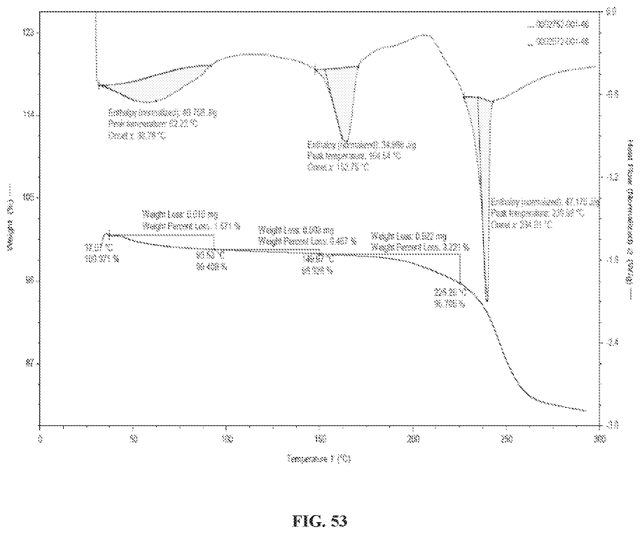

FIG. 53 depicts a DSC thermogram and TGA trace of Form C of compound A fumarate salt.

### Example 27—Form D of Compound A Fumarate Salt

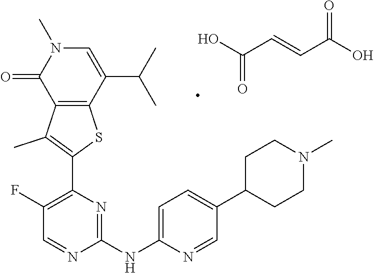 [SMILES: Cc1c(-c2nc(Nc3ccc(C4CCN(C)CC4)cn3)ncc2F)sc2c(C(C)C)cn(C)c(=O)c12 ; O=C(O)/C=C/C(=O)O]

### Form D of Compound A Fumarate Salt

Table 29 sets forth the X-ray diffraction peaks observed for Form D of compound A fumarate salt.

TABLE 29
XRPD Peak Positions for Form D of Compound A Fumarate Salt

| Position (°2θ) | Intensity cps° |
|---|---|
| 4.0 | 481 |
| 5.0 | 1647 |
| 5.6 | 1973 |
| 6.1 | 1191 |
| 9.4 | 757 |
| 11.4 | 1163 |
| 12.3 | 3245 |
| 12.7 | 1342 |
| 18.7 | 1919 |
| 24.0 | 3405 |

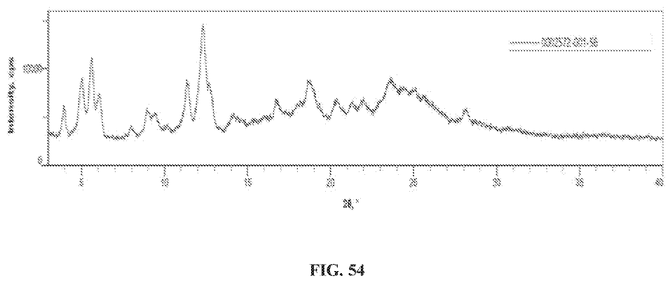

FIG. 54 depicts an XRPD pattern of Form D of compound A fumarate salt.

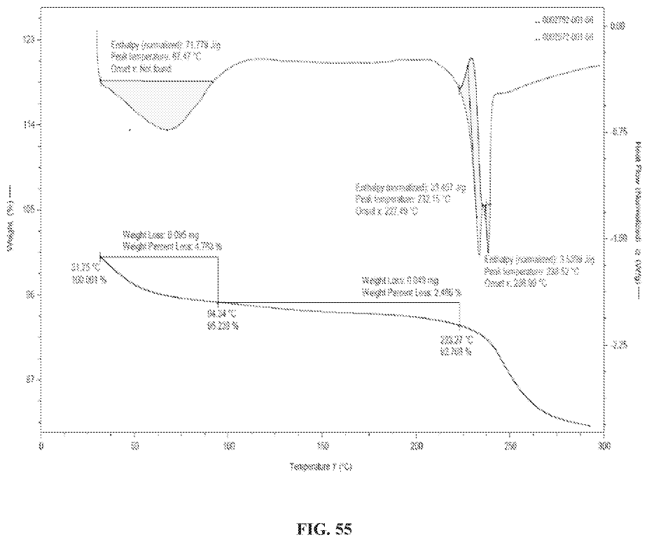

FIG. 55 depicts a DSC thermogram and TGA trace of Form D of compound A fumarate salt.

### Example 28—Form A of Compound A Citrate Salt

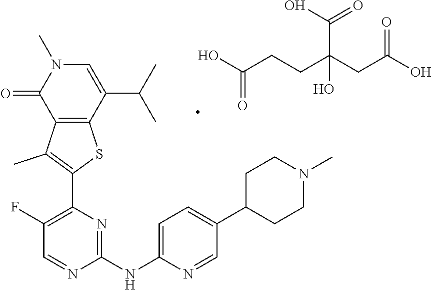 [SMILES: Cc1c(-c2nc(Nc3ccc(C4CCN(C)CC4)cn3)ncc2F)sc2c(C(C)C)cn(C)c(=O)c12 ; O=C(O)CCC(O)(CC(=O)O)C(=O)O]

### Form A of Compound A Citrate Salt

Table 30 sets forth the X-ray diffraction peaks observed for Form D of compound A citrate salt.

TABLE 30
XRPD Peak Positions for Form A of Compound A Citrate Salt

| Position (°2θ) | Intensity cps° |
|---|---|
| 12.4 | 3192 |
| 26.3 | 1495 |

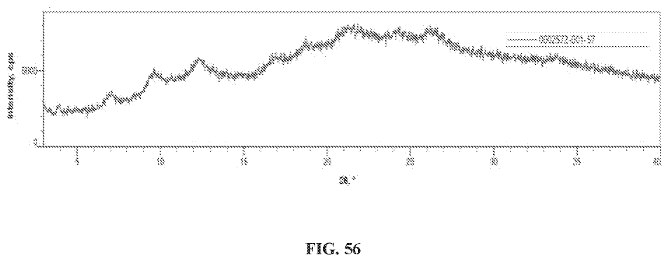

FIG. 56 depicts an XRPD pattern of Form A of compound A citrate salt.

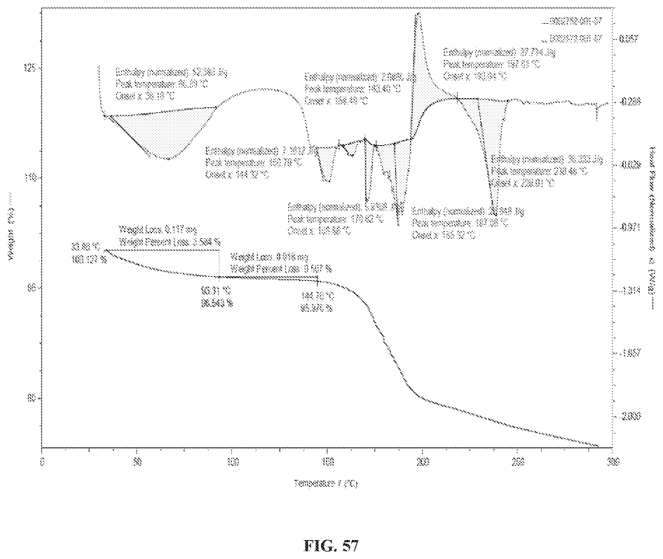

FIG. 57 depicts a DSC thermogram and TGA trace of Form A of compound A citrate salt.

### Example 29—Form a of Compound a L-Malate Salt

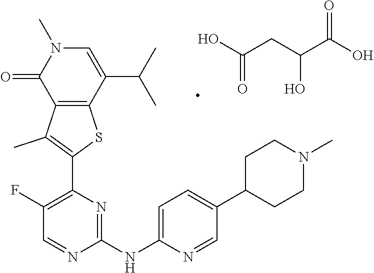 [SMILES: Cc1c(-c2nc(Nc3ccc(C4CCN(C)CC4)cn3)ncc2F)sc2c(C(C)C)cn(C)c(=O)c12 ; O=C(O)CC(O)C(=O)O]

### Form A of Compound A L-Malate Salt

Table 31 sets forth the X-ray diffraction peaks observed for Form A of compound A L-malate salt.

TABLE 31
XRPD Peak Positions for Form A of Compound A L-Malate Salt

| Position (°2θ) | Intensity cps° |
|---|---|
| 26.1 | 58801 |
| 66.0 | 985158 |

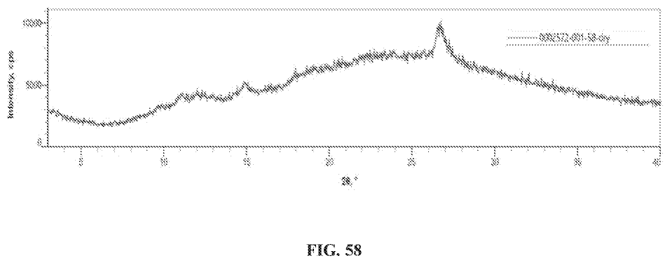

FIG. 58 depicts an XRPD pattern of Form A of compound A L-malate salt.

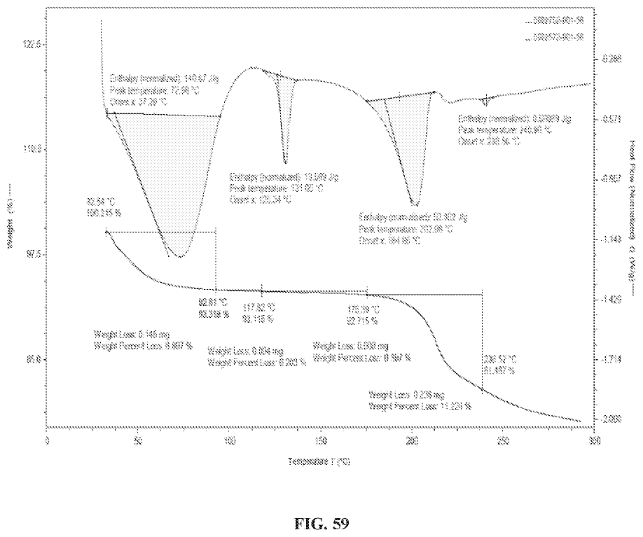

FIG. 59 depicts a DSC thermogram and TGA trace of Form A of compound A L-malate salt.

### Example 30—Form A of Compound A Mesylate Salt

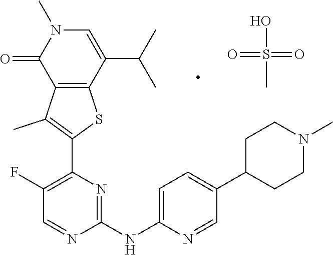

### Form A of Compound A Mesylate Salt

Table 32 sets forth the X-ray diffraction peaks observed for Form A of compound A mesylate salt.

TABLE 32
XRPD Peak Positions for Form A of Compound A Mesylate Salt

| Position (°2θ) | Intensity cps° |
|---|---|
| 10.6 | 322 |
| 21.7 | 141 |
| 23.4 | 323 |
| 26.1 | 935 |

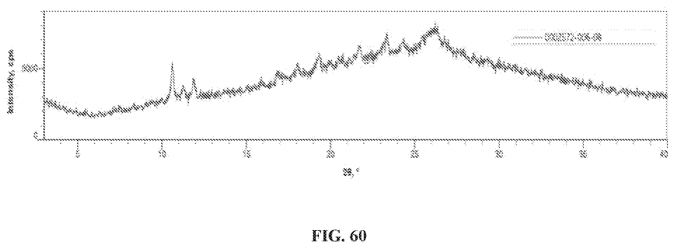

FIG. 60 depicts an XRPD pattern of Form A of compound A mesylate salt.

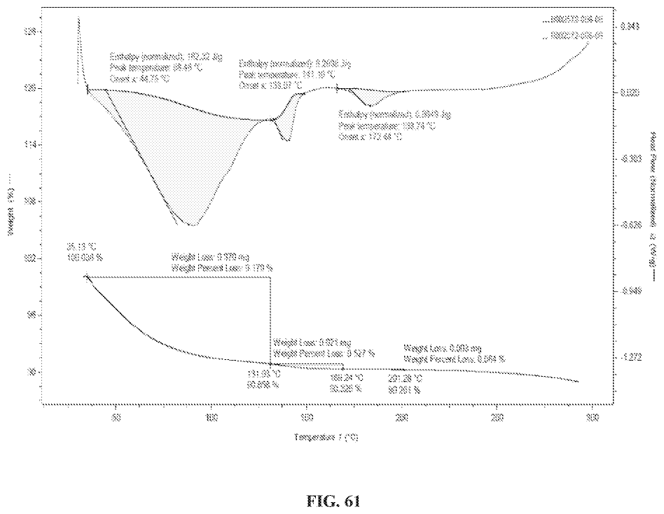

FIG. 61 depicts a DSC thermogram and TGA trace of Form A of compound A mesylate salt.

### Example 31—Form B of Compound A Mesylate Salt

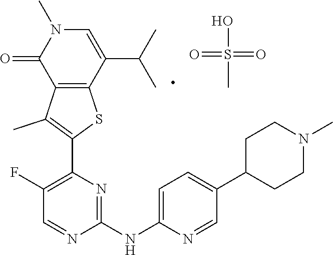

### Form B of Compound A Mesylate Salt

Table 33 sets forth the X-ray diffraction peaks observed for Form B of compound A mesylate salt.

TABLE 33
XRPD Peak Positions for Form B of Compound A Mesylate Salt

| Position (°2θ) | Intensity cps° |
|---|---|
| 10.7 | 1262 |
| 11.3 | 411 |
| 12.0 | 631 |
| 15.9 | 255 |
| 17.0 | 417 |
| 18.1 | 694 |
| 19.3 | 1147 |
| 21.8 | 881 |
| 23.4 | 1196 |
| 24.3 | 373 |

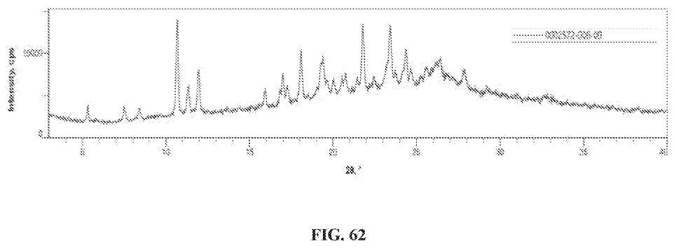

FIG. 62 depicts an XRPD pattern of Form B of compound A mesylate salt.

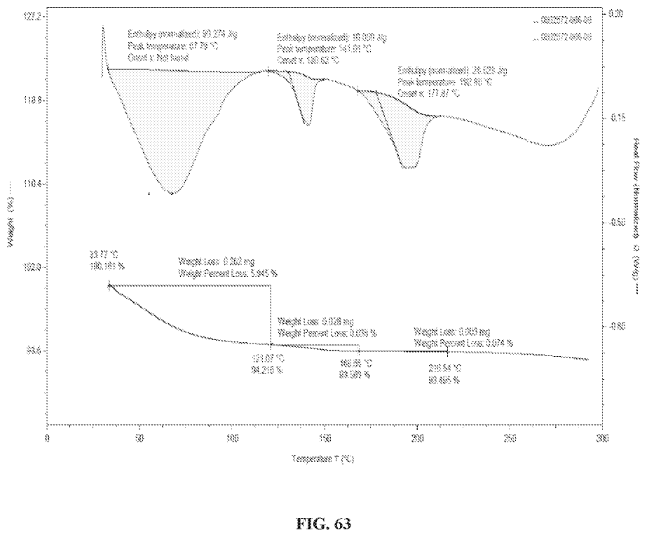

FIG. 63 depicts a DSC thermogram and TGA trace of Form B of compound A mesylate salt.

### Example 32—Form A of Compound A Esylate Salt

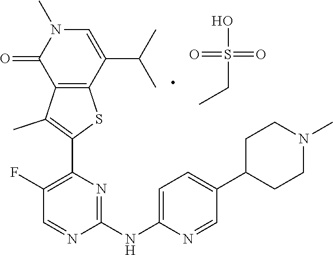

### Form A of Compound A Esylate Salt

Table 34 sets forth the X-ray diffraction peaks observed for Form A of compound A esylate salt.

TABLE 34
XRPD Peak Positions for Form A of Compound A Esylate Salt

| Position (°2θ) | Intensity cps° |
|---|---|
| 8.0 | 378 |
| 8.6 | 397 |
| 10.5 | 1452 |
| 11.2 | 323 |
| 12.4 | 547 |
| 16.5 | 506 |
| 19.9 | 1235 |
| 21.1 | 921 |
| 22.8 | 391 |
| 24.5 | 477 |

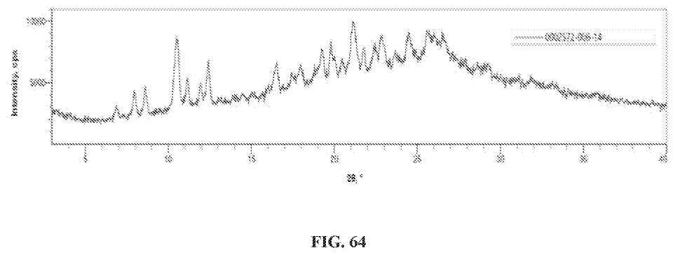

FIG. 64 depicts an XRPD pattern of Form A of compound A esylate salt.

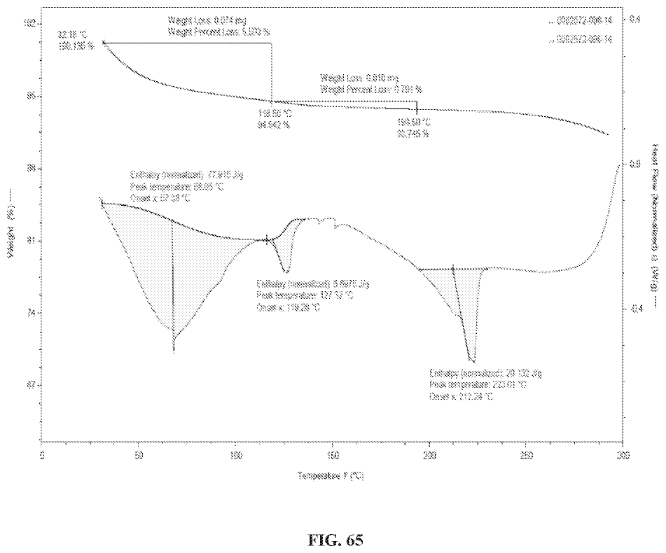

FIG. 65 depicts a DSC thermogram and TGA trace of Form A of compound A esylate salt.

### Example 33—Form B of Compound A Esylate Salt

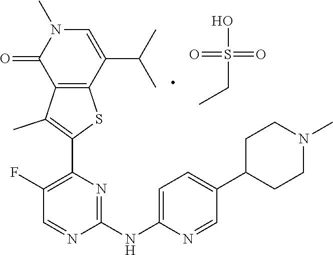

### Form B of Compound A Esylate Salt

Table 35 sets forth the X-ray diffraction peaks observed for Form B of compound A esylate salt.

TABLE 35
XRPD Peak Positions for Form B of Compound A Esylate Salt

| Position (°2θ) | Intensity cps° |
|---|---|
| 5.3 | 107 |
| 7.4 | 598 |
| 8.3 | 429 |
| 10.6 | 919 |
| 11.7 | 2359 |
| 15.6 | 267 |
| 17.6 | 186 |
| 18.8 | 767 |
| 22.9 | 328 |

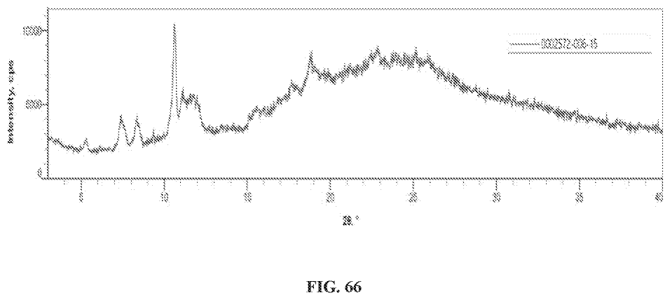

FIG. 66 depicts an XRPD pattern of Form B of compound A esylate salt.

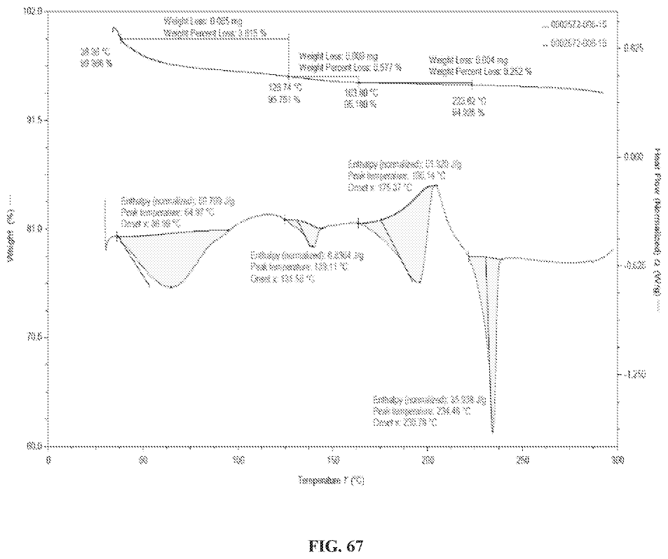

FIG. 67 depicts a DSC thermogram and TGA trace of Form B of compound A esylate salt.

### Example 34—Form A of Compound A Tosylate Salt

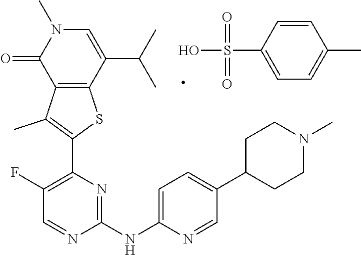

### Form A of Compound A Tosylate Salt

Table 36 sets forth the X-ray diffraction peaks observed for Form A of compound A tosylate salt.

TABLE 36
XRPD Peak Positions for Form A of Compound A Tosylate Salt

| Position (°2θ) | Intensity cps° |
|---|---|
| 5.6 | 1228 |
| 6.6 | 2832 |
| 11.4 | 1280 |
| 12.7 | 692 |
| 16.9 | 2123 |
| 17.2 | 1488 |
| 19.1 | 1009 |
| 20.8 | 1638 |
| 25.5 | 1661 |
| 26.3 | 1727 |

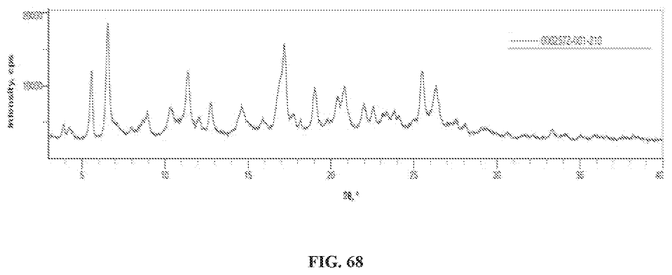

FIG. 68 depicts an XRPD pattern of Form A of compound A tosylate salt.

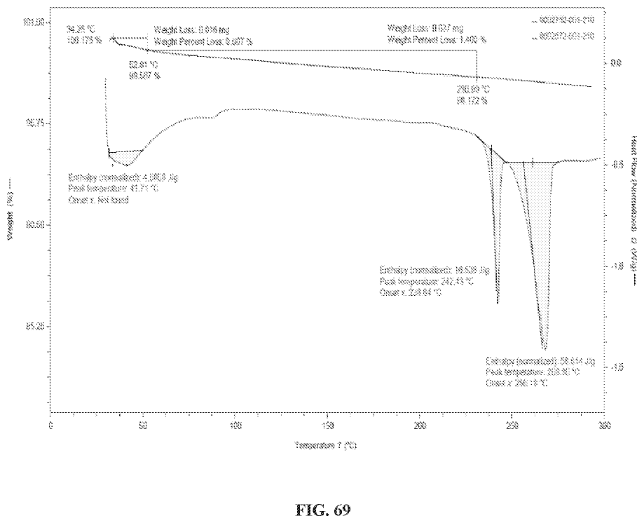

FIG. 69 depicts a DSC thermogram and TGA trace of Form A of compound A tosylate salt.

### Example 35—Form B of Compound A Tosylate Salt

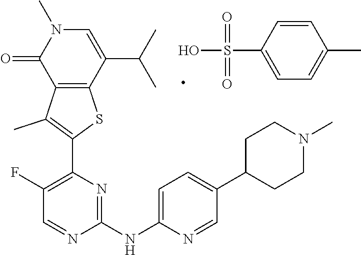

### Form B of Compound A Tosylate Salt

Table 37 sets forth the X-ray diffraction peaks observed for Form B of compound A tosylate salt.

TABLE 37
XRPD Peak Positions for Form B of Compound A Tosylate Salt

| Position (°2θ) | Intensity cps° |
|---|---|
| 5.6 | 1228 |
| 6.6 | 2832 |
| 11.4 | 1280 |
| 12.7 | 692 |
| 16.9 | 2123 |
| 17.2 | 1488 |
| 19.1 | 1009 |
| 20.8 | 1638 |
| 25.5 | 1661 |
| 26.3 | 1727 |

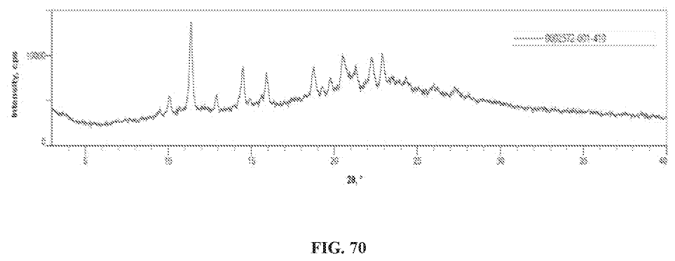

FIG. 70 depicts an XRPD pattern of Form B of compound A tosylate salt.

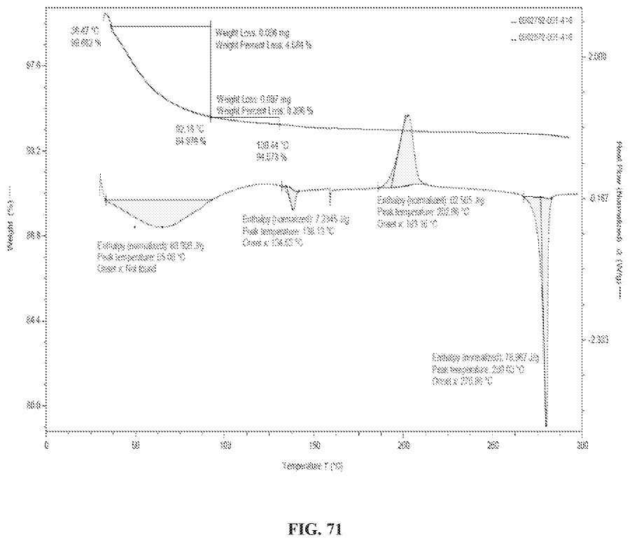

FIG. 71 depicts a DSC thermogram and TGA trace of Form B of compound A tosylate salt.

### Example 36—Form A of Compound A Glutarate Salt

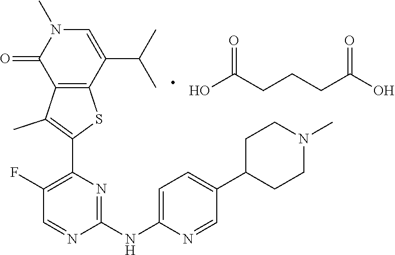

### Form A of Compound A Glutarate Salt

Table 38 sets forth the X-ray diffraction peaks observed for Form A of compound A glutarate salt.

TABLE 38
XRPD Peak Positions for Form A of Compound A Glutarate Salt

| Position (°2θ) | Intensity cps° |
|---|---|
| 8.5 | 3509 |
| 13.3 | 571 |
| 14.6 | 209 |
| 15.7 | 165 |
| 16.8 | 401 |
| 18.2 | 231 |
| 20.8 | 219 |
| 22.8 | 240 |
| 25.0 | 620 |
| 34.9 | 423 |

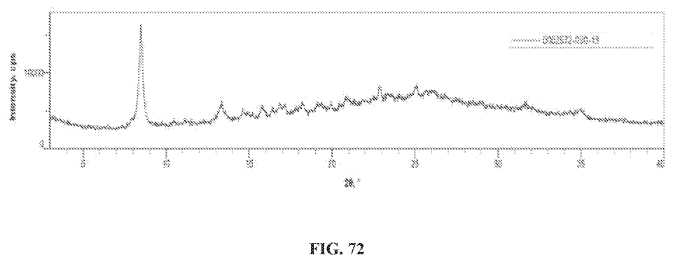

FIG. 72 depicts an XRPD pattern of Form A of compound A glutarate salt.

### Example 37—Form A of Compound A Acetate Salt

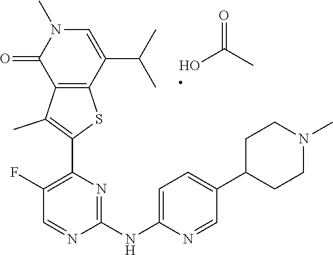

### Form A of Compound A Acetate Salt

Table 39 sets forth the X-ray diffraction peaks observed for Form A of compound A acetate salt.

TABLE 39
XRPD Peak Positions for Form A of Compound A Acetate Salt

| Position (°2θ) | Intensity cps° |
|---|---|
| 8.5 | 199 |
| 13.9 | 784 |
| 15.9 | 203 |
| 22.2 | 344 |
| 23.6 | 158 |
| 26.2 | 434 |

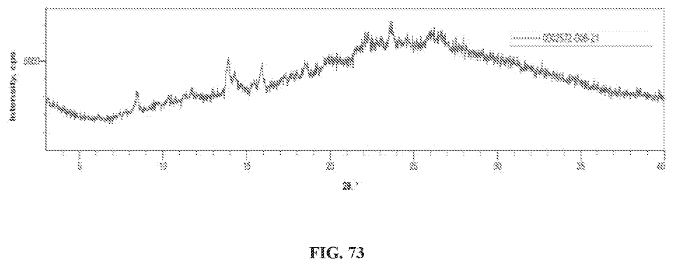

FIG. 73 depicts an XRPD pattern of Form A of compound A acetate salt.

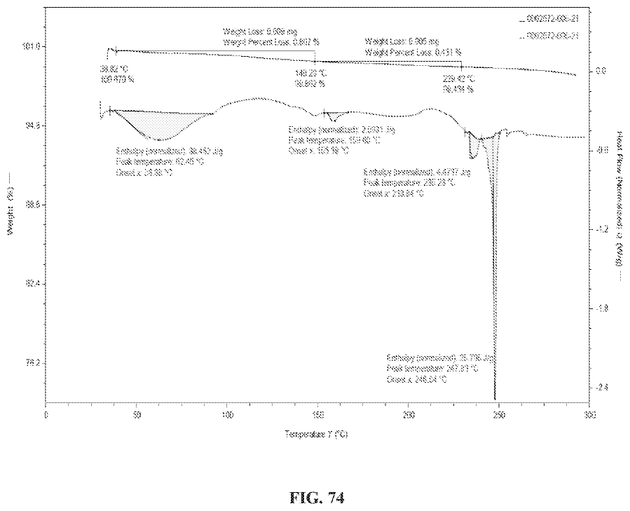

FIG. 74 depicts a DSC thermogram and TGA trace of Form A of compound A acetate salt.

### Example 38—Form A of Compound A Malonate Salt

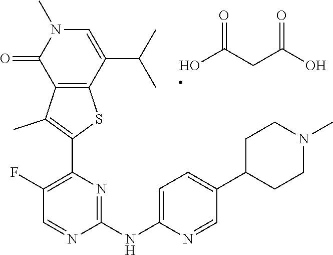

### Form A of Compound A Malonate Salt

Table 40 sets forth the X-ray diffraction peaks observed for Form A of compound A malonate salt.

TABLE 40
XRPD Peak Positions for Form A of Compound A Malonate Salt

| Position (°2θ) | Intensity cps° |
|---|---|
| 10.3 | 591 |
| 11.4 | 1530 |
| 12.6 | 892 |
| 14.6 | 464 |
| 15.2 | 1791 |
| 17.4 | 195 |
| 18.7 | 1485 |
| 20.0 | 176 |
| 21.9 | 662 |
| 26.9 | 1662 |

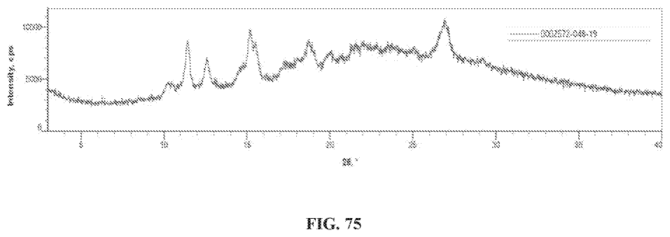

FIG. 75 depicts an XRPD pattern of Form A of compound A malonate salt.

### Example 39—Form B of Compound a Malonate Salt

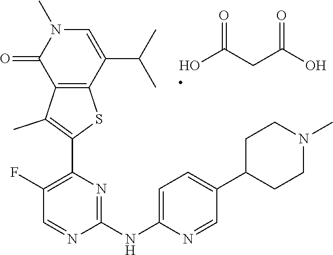

### Form B of Compound A Malonate Salt

Table 41 sets forth the X-ray diffraction peaks observed for Form B of compound A malonate salt.

TABLE 41
XRPD Peak Positions for Form B of Compound A Malonate Salt

| Position (°2θ) | Intensity cps° |
|---|---|
| 7.8 | 1768 |
| 9.6 | 3145 |
| 10.8 | 853 |
| 12.2 | 1736 |
| 12.9 | 933 |
| 14.7 | 2858 |
| 21.2 | 1217 |
| 23.0 | 1401 |
| 24.6 | 721 |
| 25.6 | 1721 |

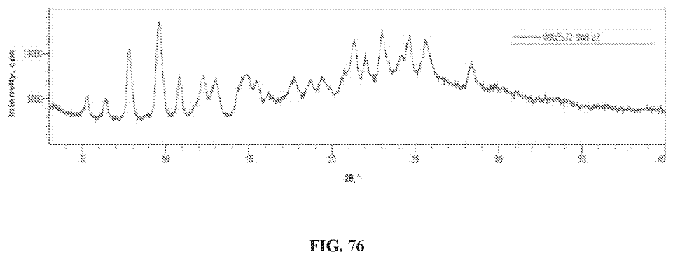

FIG. 76 depicts an XRPD pattern of Form B of compound A malonate salt.

### Example 40—Form A of Compound A Sebacate Salt

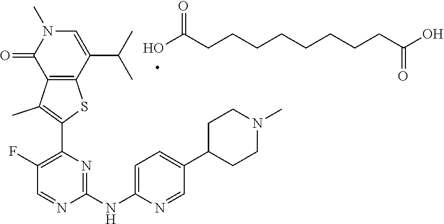

### Form A of Compound A Sebacate Salt

Table 42 sets forth the X-ray diffraction peaks observed for Form A of compound A sebacate salt.

TABLE 42
XRPD Peak Positions for Form A of Compound A Sebacate Salt

| Position (°2θ) | Intensity cps° |
|---|---|
| 7.5 | 3199 |
| 9.4 | 576 |
| 11.7 | 694 |
| 12.5 | 1362 |
| 13.5 | 982 |
| 16.9 | 2456 |
| 17.2 | 4806 |
| 19.1 | 1991 |
| 22.8 | 1186 |
| 25.6 | 740 |

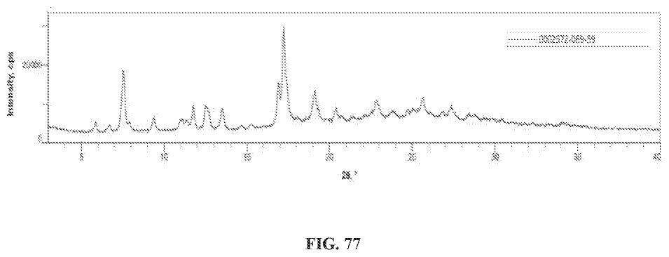

FIG. 77 depicts an XRPD pattern of Form A of compound A sebacate salt.

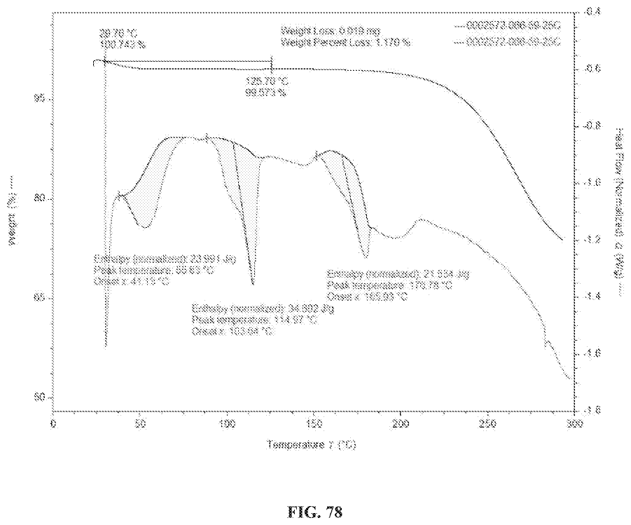

FIG. 78 depicts a DSC thermogram and TGA trace of Form A of compound A sebacate salt.

### Example 41—Form A of Compound A Aceturate Salt

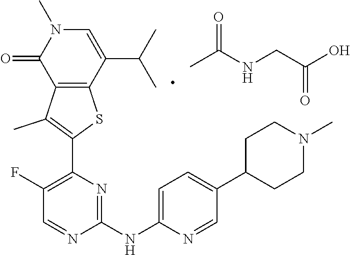

### Form A of Compound A Aceturate Salt

Table 43 sets forth the X-ray diffraction peaks observed for Form A of compound A aceturate salt.

TABLE 43
XRPD Peak Positions for Form A of Compound A Aceturate Salt

| Position (°2θ) | Intensity cps° |
|---|---|
| 13.3 | 934 |
| 14.6 | 544 |
| 15.7 | 459 |
| 16.3 | 556 |
| 17.1 | 266 |
| 18.1 | 482 |
| 19.9 | 726 |
| 21.0 | 611 |
| 22.8 | 1463 |
| 23.5 | 789 |

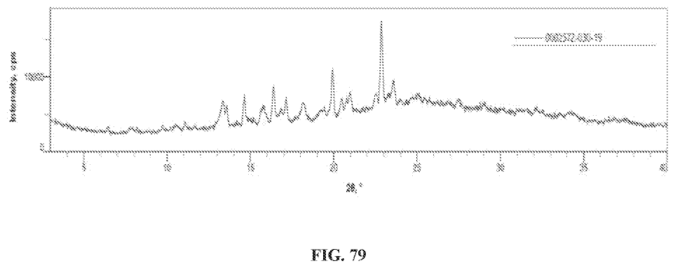

FIG. 79 depicts an XRPD pattern of Form A of compound A aceturate salt.

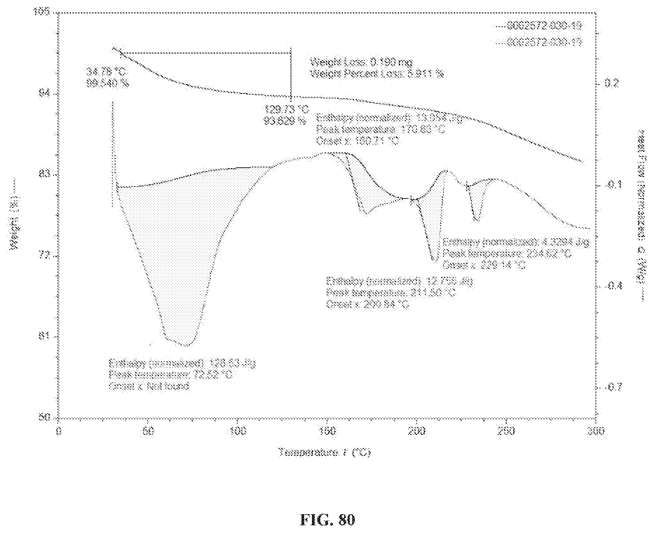

FIG. 80 depicts a DSC thermogram and TGA trace of Form A of compound A aceturate salt.

### Example 42—Form B of Compound A Aceturate Salt

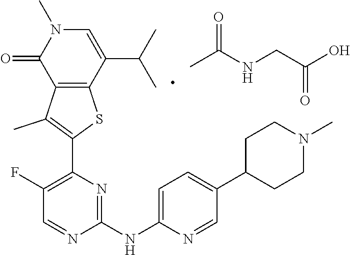

### Form B of Compound A Aceturate Salt

Table 44 sets forth the X-ray diffraction peaks observed for Form B of compound A aceturate salt.

TABLE 44
XRPD Peak Positions for Form B of Compound A Aceturate Salt

| Position (°2θ) | Intensity cps° |
|---|---|
| 14.4 | 142 |
| 15.0 | 406 |
| 16.6 | 153 |
| 18.1 | 176 |
| 21.0 | 223 |
| 21.3 | 176 |
| 21.9 | 118 |
| 27.0 | 104 |
| 29.6 | 285 |
| 38.9 | 146 |

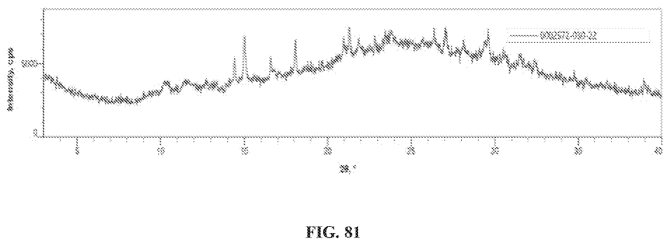

FIG. 81 depicts an XRPD pattern of Form B of compound A aceturate salt.

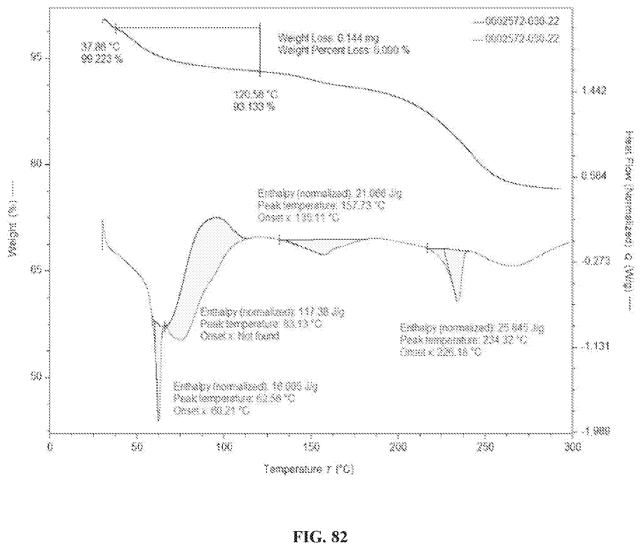

FIG. 82 depicts a DSC thermogram and TGA trace of Form B of compound A aceturate salt.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

1. Compound A: 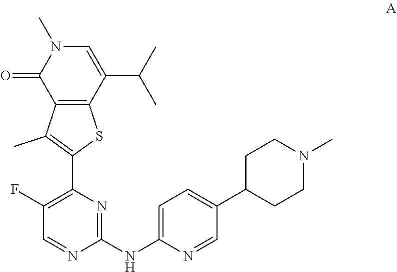 [SMILES: Cc1c(-c2nc(Nc3ccc(C4CCN(C)CC4)cn3)ncc2F)sc2c(C(C)C)cn(C)c(=O)c12]
of crystalline Form A, B or C.

2.-4. (canceled)

5. The compound according to claim 1, having one or more peaks in its XRPD selected from those at 11.5, 12.9 and 23.5±0.2 degrees theta.

6.-9. (canceled)

10. The compound according to claim 1, having one or more peaks in its XRPD selected from those at 17.8, 18.3 and 20.8±0.2 degrees theta.

11.-14. (canceled)

15. The compound according to claim 1, having one or more peaks in its XRPD selected from those at 16.4, 22.9 and 31.6±0.2 degrees theta.

16.-19. (canceled)

20. Compound A succinate salt:

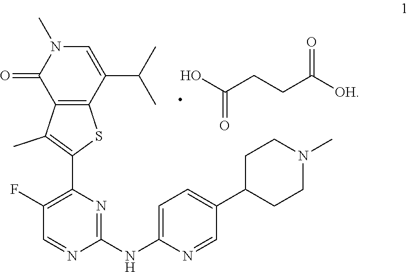

21. The compound according to claim 20, wherein said compound is crystalline.

22. (canceled)

23. (canceled)

24. The compound according to claim 21, having one or more peaks in its XRPD selected from those at 8.1, 11.6 and 24.4±0.2 degrees theta.

25.-28. (canceled)

29. The compound according to claim 21, having one or more peaks in its XRPD selected from those at 18.6, 24.1 and 24.6±0.2 degrees 2-theta.

30.-33. (canceled)

34. The compound according to claim 21, having one or more peaks in its XRPD selected from those at 9.2, 11.9 and 18.9±0.2 degrees 2-theta.

35.-38. (canceled)

39. The compound according to claim 21, having one or more peaks in its XRPD selected from those at 12.3, 19.4 and 24.7±0.2 degrees 2-theta.

40.-43. (canceled)

44. The compound according to claim 21, having one or more peaks in its XRPD selected from those at 11.9, 23.1 and 24.1±0.2 degrees 2-theta.

45.-48. (canceled)

49. The compound according to claim 21, having one or more peaks in its XRPD selected from those at 6.0, 12.5 and 24.5±0.2 degrees 2-theta.

50.-53. (canceled)

54. The compound according to claim 21, having one or more peaks in its XRPD selected from those at 6.0, 15.3 and 23.4±0.2 degrees 2-theta.

55.-58. (canceled)

59. A pharmaceutical composition comprising a compound according to claim 1, or a pharmaceutically acceptable salt thereof, and a pharmaceutically acceptable excipient.

60. (canceled)

61. A method for treating a CDK4-mediated and a CDK6-mediated disorder in a patient in need thereof, comprising administering to said patient a compound according to claim 1, or a pharmaceutically acceptable salt thereof.

62. The method according to claim 61, wherein the CDK4-mediated and CDK6-mediated disorder is a cancer.

63. The method according to claim 62, wherein the cancer is breast cancer, malignant brain tumors, colon cancer, small-cell lung cancer, non-small-cell lung cancer, bladder cancer, ovarian cancer, prostate cancer, chronic lymphoid leukemia, lymphoma, myeloma, acute myeloid leukemia, secondary pancreatic cancer or secondary brain metastases.

64. The method according to claim 63, wherein the breast cancer is HR+/HER2− or HR+/HER2+ advanced or metastatic breast cancer; and the malignant brain tumors are glioblastoma, astrocytoma, or pontine glioma.

65. (canceled)

66. (canceled)

67. The method according to claim 61, further comprising administering an additional therapeutic agent to the patient.

68. The method according to claim 67, wherein the additional therapeutic agent is a PRMT5 inhibitor, a HER2 kinase inhibitor, an aromatase inhibitor, an estrogen receptor antagonist or an alkylating agent.

69.-73. (canceled)

74. A pharmaceutical salt of Compound A: 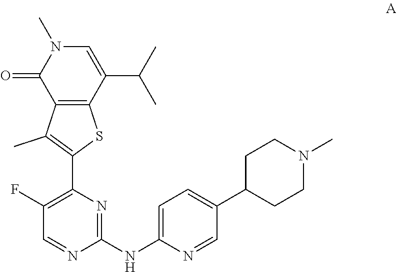 [SMILES: Cc1c(-c2nc(Nc3ccc(C4CCN(C)CC4)cn3)ncc2F)sc2c(C(C)C)cn(C)c(=O)c12]
wherein the salt is a hydrochloride, sulfate, maleate, phosphate, L-tartarate, fumarate, citrate, L-malate, tosylate, succinate, methanesulfonate, ethanesulfonate, glutarate, n-acetylglycine, acetate, malonate, or sebacate salt.

75.-108. (canceled)
